(19) United States
(12) Patent Application Publication
(10) Pub. No.: US 2026/0191952 A1
(43) Pub. Date: Jul. 9, 2026

(54) PNEUMOCOCCAL CONJUGATE VACCINE FORMULATIONS

(71) Applicant: Pfizer Inc., New York, NY (US)

(72) Inventors: Awa Diop, St. Louis, MO (US); Daniel Abbas Dixon, Arlington, MA (US); KariAnn Sweeney Efferen, Lagrangeville, NY (US); Rikhav Praful Gala, Lowell, MA (US); Anna Krasevec, Bloomingburg, NY (US); Ksenia Krylova, New Windsor, NY (US); Alex Jacob Langford, St. Louis, MO (US); Temmyn Minh Nguyen, St. Louis, MO (US); Naveen Palath, Westford, MA (US); Lynn Marie Phelan, Lake Hiawatha, NJ (US); Shuai Shi, Whippany, NJ (US); Nathan David Weiskopf, Kofu (JP); Cindy Xudong Yang, Tappan, NY (US); Sandra Irene Zeller, Hillsdale, NJ (US)

(21) Appl. No.: 19/133,544

(22) PCT Filed: Nov. 29, 2023
(86) PCT No.: PCT/IB2023/062031

§ 371 Date: May 28, 2025

Related U.S. Application Data
(60) Provisional application No. 63/385,686, filed on Dec. 1, 2022.
(60) Provisional application No. 63/582,342, filed on Sep. 13, 2023.
(60) Provisional application No. 63/599,765, filed on Nov. 16, 2023.

(51) Int. Cl. A61K 39/385; A61K 39/00; A61K 39/12; A61K 39/39

(52) U.S. Cl. CPC A61K 39/385; A61K 39/12; A61K 39/39; A61K 2039/55505

(57) ABSTRACT

The present invention relates to new vaccine formulations comprising conjugated Streptococcus pneumoniae capsular saccharide antigens (glycoconjugates) and uses thereof. Vaccine formulations of the present invention will typically comprise at least one glycoconjugate from a S. pneumoniae serotype in a formulation designed to facilitate resuspension.

### FIELD OF THE INVENTION

The present invention relates to new vaccine formulations comprising conjugated capsular saccharide antigens (glycoconjugates) and uses thereof. The formulations of the present invention will typically comprise glycoconjugates, wherein the saccharides are derived from serotypes of Streptococcus pneumoniae in a formulation of buffers, salt solutions, surfactants and adjuvants and specifically designed to facilitate resuspension of the adjuvant and/or glycoconjugates and provide long-term stability of the vaccine.

### BACKGROUND OF THE INVENTION

Infections caused by pneumococci are a major cause of morbidity and mortality all over the world. Pneumonia, febrile bacteraemia and meningitis are the most common manifestations of invasive pneumococcal disease, whereas bacterial spread within the respiratory tract may result in middle-ear infection, sinusitis or recurrent bronchitis. Compared with invasive disease, the non-invasive manifestations are usually less severe, but considerably more common.

The etiological agent of pneumococcal diseases, Streptococcus pneumoniae (pneumococcus), is a Gram-positive encapsulated coccus, surrounded by a polysaccharide capsule. Differences in the composition of this capsule permit serological differentiation between about 91 capsular types, some of which are frequently associated with pneumococcal disease, others rarely. Invasive pneumococcal infections include pneumonia, meningitis and febrile bacteraemia; among the common non-invasive manifestations are otitis media, sinusitis and bronchitis

Pneumococcal polysaccharides, in particular capsular polysaccharides, are important immunogens found on the surface of the bacteria. This has led to them being an important component in the design of pneumococcal vaccines. They have proved useful in eliciting immune responses especially when linked to carrier proteins.

Pneumococcal conjugate vaccines (PCVs) are pneumococcal vaccines used to protect against disease caused by S. pneumoniae (pneumococcus). The vaccines typically are comprised of a number of glycoconjugates derived from different serotypes of Streptococcus pneumoniae. There are currently six approved PCV vaccines: PREVNAR® (called Prevenar in some countries) (a seven-valent vaccine, e.g., comprising seven different serotypes), SYNFLORIX® (a 10-valent vaccine), PREVNAR 13® (13-valent vaccine), VAXNEUVANCE™ (a 15-valent vaccine), PREVNAR 20™ (a 20 valent vaccine), and PNEUMOVAX 23™ (a 23-valent vaccine).

One of the challenges of vaccine formulations is the sedimentation of the adjuvant and/or active ingredient (e.g., glycoconjugates) as the formulations are stored prior to administration. As the number of serotypes increase in a pneumococcal conjugate vaccine, the overall concentration of the active ingredient increases resulting in differing dispersion and sedimentation of the formulation. The formulations must be resuspended by shaking prior to administration to ensure the accuracy of the dose administered. Resuspension of the formulations is made more difficult as the number of serotypes and/or concentration of serotypes increases in the vaccine. As such, there is a need for a vaccine formulation to facilitate easier resuspension of the vaccine for administration.

### SUMMARY OF THE INVENTION

The present invention is based on the seminal discovery of vaccine formulations for pneumococcal vaccines that facilitates the resuspension of particles that have sedimented out of solution to ensure dose accuracy and long-term stability.

In an embodiment, the present invention provides formulations including at least 21 different glycoconjugates; a succinic acid or a histidine buffer having a pH in the range of 5.0 to 7.5; calcium chloride, sodium chloride and/or sodium phosphate; a surfactant; and an adjuvant.

In an embodiment, the formulation includes at least 21, 22, 23, 24, 25, 26, 27, 28, 29 or 30 different glycoconjugates. In an embodiment, the formulation is a 24-valent pneumococcal conjugate composition. In an embodiment, the formulation is a 25-valent pneumococcal conjugate composition. In one embodiment, the glycoconjugates are pneumococcal polysaccharide protein conjugates.

In an embodiment, the glycoconjugates include at least one glycoconjugate derived from Streptococcus pneumoniae serotype selected from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof. In an embodiment, the carrier protein of the glycoconjugate(s) is diphtheria cross reactive material (CRM₁₉₇), Diphtheria toxoid (DT), tetanus toxoid (TT), C5a peptidase from Streptococcus (SCP) or rhizavidin [aa 45-179J-GGGGSSS-SP1500-AAA-SP0785] (CP1).

In an embodiment, the formulation includes at least 25 glycoconjugates including at least glycoconjugates derived from S. pneumoniae serotypes 4, 6B, 9V, 14, 18C, 19F and 23F and the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 5, and 7F. In an embodiment, the S. pneumoniae serotypes 1, 4, 5, 7F, 9V and/or 23F are conjugated to PD, S. pneumoniae serotype 18C is conjugated to TT and S. pneumoniae serotype 19F is conjugated to DT.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, and 19A. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, 19A, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, 8, 10A, 11A, 12F, 15B, 19A, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 2, 3, 5, 7F, 8, 9N, 10A, 11A, 12F, 15B, 17F, 19A, 20, 22F and 33F.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 2, 3, 5, 6A, 7F, 8, 9N, 10A, 11A, 12F, 15B, 17F, 19A, 20, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In one embodiment, the S. pneumoniae serotypes are conjugated to CRM₁₉₇. In one embodiment, the S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇ and the S. pneumoniae serotype 3 is conjugated to SCP.

In an embodiment, the formulation includes at least 25 glycoconjugates including at least glycoconjugates derived from S. pneumoniae serotypes 4, 6B, 9V, 14, 18C, 19F and 23F and additionally include glycoconjugates derived from S. pneumoniae serotypes 1, 2, 3, 5, 6A, 7F, 8, 9N, 10A, 11A, 12F, 15A, 15B, 19A, 22F, 23A, 23B, 24F, 33F and 35B. In an embodiment, the S. pneumoniae serotypes are conjugated to CRM₁₉₇. In an embodiment, S. pneumoniae serotypes 1, 2, 5, 6A, 7F, 8, 9N, 10A, 11A, 12F, 15A, 15B, 19A, 22F, 23A, 23B, 24F, 33F and 35B are conjugated to CRM₁₉₇ and S. pneumoniae serotype 3 is conjugated to SCP.

In an embodiment, the formulation includes at least 25 glycoconjugates including at least S. pneumoniae serotypes 4, 6B, 9V, 14, 18C, 19F and 23F additionally include S. pneumoniae serotypes 1, 3, 5, 6A, 7F, 8, 9N, 10A, 11A, 12F, 15B, 18C, 19A, 22F and 33F. In an embodiment, at least two of the S. pneumoniae serotypes are conjugated to TT. In one embodiment, the at least two S. pneumoniae serotypes conjugated to TT are selected from S. pneumoniae serotypes 1, 3, 5, 15B and 22F. In an embodiment, at least 17 of the S. pneumoniae serotypes are conjugated to CRM₁₉₇. In an embodiment, the at least 17 S. pneumoniae serotypes conjugated to CRM₁₉₇ are selected from S. pneumoniae serotypes 1, 3, 4, 5, 6A, 6B, 7F, 8, 9N, 9V, 10A, 11A, 12F, 14, 15B, 18C, 19A, 19F 22F, 23F and 33F.

In an embodiment, the pneumococcal glycoconjugates are selected from S. pneumoniae serotype 1 conjugated to CRM_{197,} S. pneumoniae serotype 3 conjugated to CRM_{197,} S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

In an embodiment, the pneumococcal glycoconjugates are selected from S. pneumoniae serotype 1 conjugated to CRM_{197,} S. pneumoniae serotype 3 conjugated to SCP, S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

In an embodiment, the total polysaccharide concentration is about 1-100 μg per dose. In an embodiment, the concentration of polysaccharide for each serotype is about 1-10 μg per dose. In an embodiment, the buffer has a concentration of about 1-50 mM. In an embodiment, the sodium chloride has a concentration of about 1-300 mM. In an embodiment, Formulation C has a calcium chloride concentration of about 1-50 mM. In an embodiment, Formulation D has a sodium phosphate concentration of about 1-50 mM. In an embodiment, the surfactant is a polysorbate or poloxamer having a molecular weight of about 1100 Da to 17,400 Da. In an embodiment, the surfactant is polysorbate 80. In an embodiment, the surfactant is polysorbate 20. In an embodiment, the concentration of the surfactant is about of 0.001% to 1%. In an embodiment, the adjuvant is aluminum phosphate. In an embodiment, the concentration of the adjuvant is about 0.1% to 1%.

In one embodiment, the adjuvant is a liposomal adjuvant. In another embodiment, the adjuvant comprises monophosphoryl lipid A (MPLA) and a saponin. In one embodiment, the adjuvant comprises monophosphoryl lipid A phosphorylated hexaAcyl disaccharide (PHAD®) and QS-21. In one embodiment, the adjuvant is Liposomal Novel Adjuvant-1 (LiNA-1), described herein. In one embodiment, the adjuvant comprises 3D-PHAD® and QS-21. In one embodiment, the adjuvant is Liposomal Novel Adjuvant-2 (LiNA-2), described herein. In another embodiment, the adjuvant is LiNA-2A, described herein. In yet another embodiment, the adjuvant is LiNA-2B, described herein. In other embodiments, the formulation comprises more than one adjuvant. In a particular embodiment, the formulation comprises aluminum phosphate and LiNA-2.

In an embodiment, the present invention provides formulations including at least 21 different glycoconjugates; a succinic acid buffer having a pH in the range of 5.0 to 7.5; calcium chloride; sodium chloride; a surfactant; and an adjuvant.

In an embodiment, the formulation includes at least 21, 22, 23, 24, 25, 26, 27, 28, 29 or 30 different glycoconjugates. In an embodiment, the formulation is a 24-valent pneumococcal conjugate composition. In an embodiment, the formulation is a 25-valent pneumococcal conjugate composition. In one embodiment, the glycoconjugates are pneumococcal polysaccharide protein conjugates.

In an embodiment, the glycoconjugates include at least one glycoconjugate derived from Streptococcus pneumoniae serotype selected from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof. In an embodiment, the carrier protein of the glycoconjugate(s) is diphtheria cross reactive material (CRM₁₉₇), Diphtheria toxoid (DT), tetanus toxoid (TT), C5a peptidase from Streptococcus (SCP) or rhizavidin [aa 45-179J-GGGGSSS-SP1500-AAA-SP0785] (CP1).

In an embodiment, the formulation includes at least 25 glycoconjugates including at least glycoconjugates derived from S. pneumoniae serotypes 4, 6B, 9V, 14, 18C, 19F and 23F and the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 5, and 7F. In an embodiment, the S. pneumoniae serotypes 1, 4, 5, 7F, 9V and/or 23F are conjugated to PD, S. pneumoniae serotype 18C is conjugated to TT and S. pneumoniae serotype 19F is conjugated to DT.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, and 19A. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, 19A, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, 8, 10A, 11A, 12F, 15B, 19A, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 2, 3, 5, 7F, 8, 9N, 10A, 11A, 12F, 15B, 17F, 19A, 20, 22F and 33F.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 2, 3, 5, 6A, 7F, 8, 9N, 10A, 11A, 12F, 15B, 17F, 19A, 20, 22F and 33F. In one embodiment, the S. pneumoniae serotypes are conjugated to CRM_{197.}

In an embodiment, the formulation includes at least 25 glycoconjugates including at least glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In one embodiment, the S. pneumoniae serotypes are conjugated to CRM₁₉₇. In one embodiment, the S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇ and the S. pneumoniae serotype 3 is conjugated to SCP.

In an embodiment, the formulation includes at least 25 glycoconjugates including at least glycoconjugates derived from S. pneumoniae serotypes 4, 6B, 9V, 14, 18C, 19F and 23F and additionally include glycoconjugates derived from S. pneumoniae serotypes 1, 2, 3, 5, 6A, 7F, 8, 9N, 10A, 11A, 12F, 15A, 15B, 19A, 22F, 23A, 23B, 24F, 33F and 35B. In an embodiment, the S. pneumoniae serotypes are conjugated to CRM₁₉₇. In an embodiment, S. pneumoniae serotypes 1, 2, 5, 6A, 7F, 8, 9N, 10A, 11A, 12F, 15A, 15B, 19A, 22F, 23A, 23B, 24F, 33F and 35B are conjugated to CRM₁₉₇ and S. pneumoniae serotype 3 is conjugated to SCP.

In an embodiment, the formulation includes at least 25 glycoconjugates including at least S. pneumoniae serotypes 4, 6B, 9V, 14, 18C, 19F and 23F additionally include S. pneumoniae serotypes 1, 3, 5, 6A, 7F, 8, 9N, 10A, 11A, 12F, 15B, 18C, 19A, 22F and 33F. In an embodiment, at least two of the S. pneumoniae serotypes are conjugated to TT. In one embodiment, the at least two S. pneumoniae serotypes conjugated to TT are selected from S. pneumoniae serotypes 1, 3, 5, 15B and 22F. In an embodiment, at least 17 of the S. pneumoniae serotypes are conjugated to CRM₁₉₇. In an embodiment, the at least 17 S. pneumoniae serotypes conjugated to CRM₁₉₇ are selected from S. pneumoniae serotypes 1, 3, 4, 5, 6A, 6B, 7F, 8, 9N, 9V, 10A, 11A, 12F, 14, 15B, 18C, 19A, 19F 22F, 23F and 33F.

In an embodiment, the pneumococcal glycoconjugates are selected from S. pneumoniae serotype 1 conjugated to CRM_{197,} S. pneumoniae serotype 3 conjugated to CRM_{197,} S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

In an embodiment, the pneumococcal glycoconjugates are selected from S. pneumoniae serotype 1 conjugated to CRM_{197,} S. pneumoniae serotype 3 conjugated to SCP, S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

In an embodiment, the total polysaccharide concentration is about 1-100 μg per dose. In an embodiment, the concentration of polysaccharide for each serotype is about 1-10 μg per dose. In an embodiment, the buffer has a concentration of about 1-50 mM. In an embodiment, the sodium chloride has a concentration of about 1-300 mM. In an embodiment, the surfactant is a polysorbate or poloxamer having a molecular weight of about 1100 Da to 17,400 Da. In an embodiment, the surfactant is polysorbate 80. In an embodiment, the surfactant is polysorbate 20. In an embodiment, the concentration of the surfactant is about of 0.001% to 1%. In an embodiment, the adjuvant is aluminum phosphate. In an embodiment, the concentration of the adjuvant is about 0.1% to 1%. In another embodiment, the concentration of the adjuvant is between about 0.01% and about 0.1%. In yet another embodiment, the concentration of the adjuvant is between about 0.1 and about 1.0 mg/mL. In one embodiment, the concentration of the adjuvant is about 0.025%. In a particular embodiment, the adjuvant is aluminum phosphate at a concentration of about 0.025%.

In an embodiment, the present invention provides formulations including at least 21 different glycoconjugates; a succinic acid buffer having a pH in the range of 5.0 to 7.5; sodium chloride; sodium phosphate; a surfactant; and an adjuvant.

In an embodiment, the formulation includes at least 21, 22, 23, 24, 25, 26, 27, 28, 29 or 30 different glycoconjugates. In an embodiment, the formulation is a 24-valent pneumococcal conjugate composition. In an embodiment, the formulation is a 25-valent pneumococcal conjugate composition. In one embodiment, the glycoconjugates are pneumococcal polysaccharide protein conjugates.

In an embodiment, the glycoconjugates include at least one glycoconjugate derived from Streptococcus pneumoniae serotype selected from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof. In an embodiment, the carrier protein of the glycoconjugate(s) is diphtheria cross reactive material (CRM₁₉₇), Diphtheria toxoid (DT), tetanus toxoid (TT), C5a peptidase from Streptococcus (SCP) or rhizavidin [aa 45-179J-GGGGSSS-SP1500-AAA-SP0785] (CP1).

In an embodiment, the formulation includes at least 25 glycoconjugates including at least glycoconjugates derived from S. pneumoniae serotypes 4, 6B, 9V, 14, 18C, 19F and 23F and the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 5, and 7F. In an embodiment, the S. pneumoniae serotypes 1, 4, 5, 7F, 9V and/or 23F are conjugated to PD, S. pneumoniae serotype 18C is conjugated to TT and S. pneumoniae serotype 19F is conjugated to DT.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, and 19A. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, 19A, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, 8, 10A, 11A, 12F, 15B, 19A, 22F and 33F. In one embodiment, the S. pneumoniae serotypes are conjugated to CRM_{197.}

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 2, 3, 5, 7F, 8, 9N, 10A, 11A, 12F, 15B, 17F, 19A, 20, 22F and 33F.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 2, 3, 5, 6A, 7F, 8, 9N, 10A, 11A, 12F, 15B, 17F, 19A, 20, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In one embodiment, the S. pneumoniae serotypes are conjugated to CRM₁₉₇. In one embodiment, the S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇ and the S. pneumoniae serotype 3 is conjugated to SCP.

In an embodiment, the formulation includes at least 25 glycoconjugates including at least glycoconjugates derived from S. pneumoniae serotypes 4, 6B, 9V, 14, 18C, 19F and 23F and additionally include glycoconjugates derived from S. pneumoniae serotypes 1, 2, 3, 5, 6A, 7F, 8, 9N, 10A, 11A, 12F, 15A, 15B, 19A, 22F, 23A, 23B, 24F, 33F and 35B. In an embodiment, the S. pneumoniae serotypes are conjugated to CRM₁₉₇. In an embodiment, S. pneumoniae serotypes 1, 2, 5, 6A, 7F, 8, 9N, 10A, 11A, 12F, 15A, 15B, 19A, 22F, 23A, 23B, 24F, 33F and 35B are conjugated to CRM₁₉₇ and S. pneumoniae serotype 3 is conjugated to SCP.

In an embodiment, the formulation includes at least 25 glycoconjugates including at least S. pneumoniae serotypes 4, 6B, 9V, 14, 18C, 19F and 23F additionally include S. pneumoniae serotypes 1, 3, 5, 6A, 7F, 8, 9N, 10A, 11A, 12F, 15B, 18C, 19A, 22F and 33F. In an embodiment, at least two of the S. pneumoniae serotypes are conjugated to TT. In one embodiment, the at least two S. pneumoniae serotypes conjugated to TT are selected from S. pneumoniae serotypes 1, 3, 5, 15B and 22F. In an embodiment, at least 17 of the S. pneumoniae serotypes are conjugated to CRM₁₉₇. In an embodiment, the at least 17 S. pneumoniae serotypes conjugated to CRM₁₉₇ are selected from S. pneumoniae serotypes 1, 3, 4, 5, 6A, 6B, 7F, 8, 9N, 9V, 10A, 11A, 12F, 14, 15B, 18C, 19A, 19F 22F, 23F and 33F.

In an embodiment, the pneumococcal glycoconjugates are selected from S. pneumoniae serotype 1 conjugated to CRM_{197,} S. pneumoniae serotype 3 conjugated to CRM_{197,} S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

In an embodiment, the pneumococcal glycoconjugates are selected from S. pneumoniae serotype 1 conjugated to CRM_{197,} S. pneumoniae serotype 3 conjugated to SCP, S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

In an embodiment, the total polysaccharide concentration is about 1-100 μg per dose. In an embodiment, the concentration of polysaccharide for each serotype is about 1-10 μg per dose. In an embodiment, the buffer has a concentration of about 1-50 mM. In an embodiment, the sodium chloride has a concentration of about 1-300 mM. In an embodiment, the calcium chloride concentration is about 1-50 mM. In an embodiment, the surfactant is a polysorbate or poloxamer having a molecular weight of about 1100 Da to 17,400 Da. In an embodiment, the surfactant is polysorbate 80. In an embodiment, the surfactant is polysorbate 20. In an embodiment, the concentration of the surfactant is about of 0.001% to 1%. In an embodiment, the adjuvant is aluminum phosphate. In an embodiment, the concentration of the adjuvant is about 0.1% to 1%.

In an embodiment, the present invention provides formulations including at least 21 different glycoconjugates; a histidine buffer having a pH in the range of 5.0 to 7.5; sodium chloride; a surfactant; and an adjuvant.

In an embodiment, the formulation includes at least 21, 22, 23, 24, 25, 26, 27, 28, 29 or 30 different glycoconjugates. In an embodiment, the formulation is a 24-valent pneumococcal conjugate composition. In an embodiment, the formulation is a 25-valent pneumococcal conjugate composition. In one embodiment, the glycoconjugates are pneumococcal polysaccharide protein conjugates.

In an embodiment, the glycoconjugates include at least one glycoconjugate derived from Streptococcus pneumoniae serotype selected from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof. In an embodiment, the carrier protein of the glycoconjugate(s) is diphtheria cross reactive material (CRM₁₉₇), Diphtheria toxoid (DT), tetanus toxoid (TT), C5a peptidase from Streptococcus (SCP) or rhizavidin [aa 45-179J-GGGGSSS-SP1500-AAA-SP0785] (CP1).

In an embodiment, the formulation includes at least 25 glycoconjugates including at least glycoconjugates derived from S. pneumoniae serotypes 4, 6B, 9V, 14, 18C, 19F and 23F and the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 5, and 7F. In an embodiment, the S. pneumoniae serotypes 1, 4, 5, 7F, 9V and/or 23F are conjugated to PD, S. pneumoniae serotype 18C is conjugated to TT and S. pneumoniae serotype 19F is conjugated to DT.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, and 19A. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, 19A, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 3, 5, 6A, 7F, 8, 10A, 11A, 12F, 15B, 19A, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 2, 3, 5, 7F, 8, 9N, 10A, 11A, 12F, 15B, 17F, 19A, 20, 22F and 33F.

In an embodiment, the formulation includes at least 25 glycoconjugates including at least glycoconjugates derived from S. pneumoniae serotypes 4, 6B, 9V, 14, 18C, 19F and 23F and additionally include S. pneumoniae serotypes 1, 2, 3, 5, 6A, 7F, 8, 9N, 10A, 11A, 12F, 15B, 17F, 19A, 20, 22F and 33F. In one embodiment, the S. pneumoniae serotypes are conjugated to CRM₁₉₇.

In an embodiment, the formulation includes at least 25 glycoconjugates including at least glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In one embodiment, the S. pneumoniae serotypes are conjugated to CRM₁₉₇. In one embodiment, the S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇ and the S. pneumoniae serotype 3 is conjugated to SCP.

In an embodiment, the formulation includes at least 25 glycoconjugates including at least glycoconjugates derived from S. pneumoniae serotypes 4, 6B, 9V, 14, 18C, 19F and 23F and additionally include glycoconjugates derived from S. pneumoniae serotypes 1, 2, 3, 5, 6A, 7F, 8, 9N, 10A, 11A, 12F, 15A, 15B, 19A, 22F, 23A, 23B, 24F, 33F and 35B. In an embodiment, the S. pneumoniae serotypes are conjugated to CRM₁₉₇. In an embodiment, S. pneumoniae serotypes 1, 2, 5, 6A, 7F, 8, 9N, 10A, 11A, 12F, 15A, 15B, 19A, 22F, 23A, 23B, 24F, 33F and 35B are conjugated to CRM₁₉₇ and S. pneumoniae serotype 3 is conjugated to SCP.

In an embodiment, the formulation includes at least 25 glycoconjugates including at least S. pneumoniae serotypes 4, 6B, 9V, 14, 18C, 19F and 23F additionally include S. pneumoniae serotypes 1, 3, 5, 6A, 7F, 8, 9N, 10A, 11A, 12F, 15B, 18C, 19A, 22F and 33F. In an embodiment, at least two of the S. pneumoniae serotypes are conjugated to TT. In one embodiment, the at least two S. pneumoniae serotypes conjugated to TT are selected from S. pneumoniae serotypes 1, 3, 5, 15B and 22F. In an embodiment, at least 17 of the S. pneumoniae serotypes are conjugated to CRM₁₉₇. In an embodiment, the at least 17 S. pneumoniae serotypes conjugated to CRM₁₉₇ are selected from S. pneumoniae serotypes 1, 3, 4, 5, 6A, 6B, 7F, 8, 9N, 9V, 10A, 11A, 12F, 14, 15B, 18C, 19A, 19F 22F, 23F and 33F.

In an embodiment, the pneumococcal glycoconjugates are selected from S. pneumoniae serotype 1 conjugated to CRM_{197,} S. pneumoniae serotype 3 conjugated to CRM_{197,} S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

In an embodiment, the pneumococcal glycoconjugates are selected from S. pneumoniae serotype 1 conjugated to CRM_{197,} S. pneumoniae serotype 3 conjugated to SCP, S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

In an embodiment, the total polysaccharide concentration is about 1-100 μg per dose. In an embodiment, the concentration of polysaccharide for each serotype is about 1-10 μg per dose. In an embodiment, the buffer has a concentration of about 1-50 mM. In an embodiment, the sodium chloride has a concentration of about 1-300 mM. In an embodiment, In an embodiment, the sodium phosphate concentration is about 1-50 mM. In an embodiment, the surfactant is a polysorbate or poloxamer having a molecular weight of about 1100 Da to 17,400 Da. In an embodiment, the surfactant is polysorbate 80. In an embodiment, the surfactant is polysorbate 20. In an embodiment, the concentration of the surfactant is about of 0.001% to 1%. In an embodiment, the adjuvant is aluminum phosphate. In an embodiment, the concentration of the adjuvant is about 0.1% to 1%.

In an embodiment, the formulation includes 25 glycoconjugates, 5 mM succinate pH 5.8, 150 mM sodium chloride, 20 mM calcium chloride, 0.02% polysorbate 80 and 0.25 mg/ml aluminum phosphate. In an embodiment, the 25 glycoconjugates include glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.

In an embodiment, the formulation includes 25 glycoconjugates, 5 mM succinate pH 5.8, 40 mM sodium phosphate, 245 mM sodium chloride, 0.02% polysorbate 80 and 0.25 mg/ml aluminum phosphate. In an embodiment, the 25 glycoconjugates include glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.

In an embodiment, the formulation includes 25 glycoconjugates, 25 mM Histidine pH 5.8, 245 mM sodium chloride, 0.02% polysorbate 80 and 0.25 mg/ml aluminum phosphate. In an embodiment, the 25 glycoconjugates include glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.

In an embodiment, the present invention provides a composition including at least 25 different glycoconjugates and insoluble aluminum phosphate adjuvant, wherein at time T₀ substantially all of the at least 25 different glycoconjugates are dissolved in a liquid phase or adsorbed to the insoluble aluminum phosphate adjuvant as fully dispersed liquid suspension and the at least 25 different glycoconjugates are at a concentration C₀ in the liquid phase; at time T₁ a portion of the at least 25 different glycoconjugates adsorbed to the insoluble adjuvant sediment from the liquid phase to form a sediment and the at least 25 different glycoconjugates are at a concentration C1 in the liquid phase; at time T₂ additional portion of the at least 25 different glycoconjugates adsorbed to the insoluble adjuvant sediment from the liquid phase to form a sediment and the at least 25 different glycoconjugates are at a concentration C2 in the liquid phase; and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the height of a flat-bottomed cylindrical glass sample cell, collecting precipitate data every 20 μm.

In an embodiment, T₀ is 0 hour. In an embodiment, T₁ is about 0.01 hours to 4 hours. In an embodiment, T₁ is about 1 hour to 2 hours. In an embodiment, T₂ is about 1 hour to 5 hours. In an embodiment, T₂ is about 4 hours. In an embodiment, C₀ is greater than C₁ and C₂. In an embodiment, C₁ is greater than C₂.

In an embodiment, at T₁ peak thickness of the sedimentation front is about 0 mm to 20 mm. In an embodiment, at T₁ peak thickness of the sedimentation front is at least 2 mm. In an embodiment, at T₂ peak thickness of the sedimentation front is about 2 mm to 25 mm. In an embodiment, at T₂ peak thickness of the sedimentation front is at least 10 mm. In an embodiment, the sedimentary velocity of the sedimentation front is less than a peak thickness of 10 mm at about 1 hour and is greater than a peak thickness of 18 mm at about 4 hours.

In an embodiment, the invention further includes a time T₃ wherein the insoluble aluminum phosphate adsorbed glycoconjugate sedimentation is at equilibrium with the liquid phase at T₃. In an embodiment, T₃ is about 2 hours to 5 hours. In an embodiment, at T₃ peak thickness of the sedimentation front is about 25 mm to 35 mm. In an embodiment, the composition has been at rest for about 1 month. In an embodiment, the composition has been at rest for at least 2 weeks. In an embodiment, the composition is stored in a container. In an embodiment, the container is a syringe. In an embodiment, where after T₃ the composition is resuspended with 1-10 handshakes. In an embodiment, after T₃ the composition is resuspended with 1 handshake. In an embodiment, the composition comprises the formulation previously described.

In an embodiment, the present invention provides a liquid filled container including at least 25 different glycoconjugates and insoluble aluminum phosphate adjuvant, wherein: at time T₀, substantially all of the at least 25 different glycoconjugates are dissolved in a liquid phase or adsorbed to the insoluble aluminum phosphate adjuvant as fully dispersed liquid suspension and the at least 25 different glycoconjugates are at a concentration C₀ in the liquid phase; at time T₁, a portion of the at least 25 different glycoconjugates adsorbed to the insoluble adjuvant sediment from the liquid phase to form a sediment and the at least 25 different glycoconjugates has a concentration C₁ in the liquid phase; at time T₂ additional portion of the at least 25 different glycoconjugates adsorbed to the insoluble adjuvant sediment from the liquid phase to form a sediment and the at least 25 different glycoconjugates are at a concentration C₂ in the liquid phase; and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the thickness of a flat-bottomed cylindrical glass sample cell, collecting precipitate data every 20 μm.

In an embodiment, T₀ is 0 hour. In an embodiment, T₁ is about 0.01 hours to 4 hours. In an embodiment, T₁ is about 1 hour to 2 hours. In an embodiment, T₂ is about 1 hour to 5 hours. In an embodiment, T₂ is about 4 hours. In an embodiment, C₀ is greater than C₁ and C₂. In an embodiment, C₁ is greater than C₂.

In an embodiment, at T₁ peak thickness of the sedimentation front is about 0 mm to 20 mm. In an embodiment, at T₁ peak thickness of the sedimentation front is at least 2 mm. In an embodiment, at T₂ peak thickness of the sedimentation front is about 2 mm to 25 mm. In an embodiment, at T₂ peak thickness of the sedimentation front is at least 10 mm.

In an embodiment, the invention further includes a time T₃ wherein the insoluble aluminum phosphate adsorbed glycoconjugate sedimentation is at equilibrium with the liquid phase at T₃. In an embodiment, T₃ is about 2 hours to 5 hours. In an embodiment, at T₃ peak thickness of the sedimentation front is about 25 mm to 35 mm. In an embodiment, the container has been at rest for about 1 month. In an embodiment, the container has been at rest for at least 2 weeks. In an embodiment, the container is a syringe. In an embodiment, after T₃ the composition is resuspended with 1 to 10 handshakes. In an embodiment, after T₃ the composition is resuspended with 1 handshake. In an embodiment, the liquid comprises the formulation previously described.

### BRIEF DESCRIPTION OF THE DRAWINGS

FIG. 1 shows the sedimentation velocity of different vaccine formulations by plotting the peak thickness (also known as the sedimentation front) as a function of time (hr).

FIG. 2 shows the area on the graph (shaded) indicating between the seven serotype control formulation sedimentation curve and the 20 serotype control formulation sedimentation curve.

FIG. 3 shows the area on the graph (shaded) indicating between the seven serotype control formulation sedimentation curve and the 25 serotype control formulation sedimentation curve.

FIG. 4 shows the sedimentation cake height of the different vaccine formulations.

FIG. 5 shows resuspension of the different formulations after resting for 3 days or 2 weeks.

FIG. 6 graphically depicts the number of handshakes required to resuspend the tested samples in a pre-filled syringe (PFS) after time points of 2 days, 7 days, and 30 days after storage of the syringe. The samples tested included samples with and without LiNA-2A (as discussed in Example 6).

FIG. 7 graphically depicts the number of handshakes required to resuspend the tested samples in a pre-filled syringe (PFS) after time points of 0 days, 7 days, and 30 days after storage of the syringe. The samples tested included samples with and without LiNA-1 (as discussed in Example 6).

### DETAILED DESCRIPTION OF THE INVENTION

The present invention is based on the seminal discovery of vaccine formulations for pneumococcal vaccines that facilitates the resuspension of particles that have sedimented out of the liquid phase to ensure dose accuracy and long-term stability.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

### Vaccine Formulations

In some embodiments, vaccine formulations of the present disclosure comprise one or more of the following immunogens: naturally-occurring or artificially-created proteins, recombinant proteins, glycoproteins, peptides, carbohydrates, saccharides, nucleic acids, haptens, whole viruses, bacteria, protozoa, or virus-like particles, or conjugates thereof. Exemplary nucleic acids or polynucleotides of the vaccine formulations include, but are not limited to, ribonucleic acids (RNAs), including mRNA, and deoxyribonucleic acids (DNAs). In some embodiments, the vaccine formulations include DNA encoding a polypeptide or fragment thereof described herein. In some embodiments, the vaccine formulations include RNA encoding a polypeptide or fragment thereof described herein. In some embodiments, the vaccine formulations include an mRNA polynucleotide encoding a polypeptide or fragment thereof described herein. In some embodiments, the vaccine formulations comprise a modified RNA molecule (modRNA).

In some embodiments, vaccine formulations of the present disclosure comprise capsular saccharide antigens, optionally wherein the capsular saccharides are conjugated. Vaccine formulations of the present invention will typically comprise conjugated capsular saccharide antigens (also named glycoconjugates), wherein the saccharides are derived from serotypes of S. pneumoniae.

Preferably, the number of S. pneumoniae capsular saccharides is at least 25 different serotypes (or “v”, valences, “25v”). In one embodiment there are 21 different serotypes. In one embodiment there are 22 different serotypes. In one embodiment there are 23 different serotypes. In one embodiment there are 24 different serotypes. In one embodiment there are 25 different serotypes. In one embodiment there are 26 different serotypes. In one embodiment there are 27 different serotypes. In one embodiment there are 28 different serotypes. In an embodiment there are 29 different serotypes. In an embodiment there are 30 different serotypes. In an embodiment there are 31 different serotypes. In one embodiment there are 32 different serotypes. In one embodiment there are 32 different serotypes. In one embodiment there are 33 different serotypes. In one embodiment there are 34 different serotypes. In an embodiment there are 35 different serotypes. The capsular saccharides are conjugated to a carrier protein to form glycoconjugates as described here below.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein.

For the purposes of the invention the term ‘glycoconjugate’ indicates a capsular saccharide either linked covalently or via a high affinity interaction to a carrier protein. In one embodiment a capsular saccharide is linked directly to a carrier protein. In a second embodiment, the capsular saccharide is linked to a protein through a spacer/linker.

### Carrier Proteins

In a preferred embodiment, the carrier protein of the glycoconjugates is selected in the group consisting of: DT (Diphtheria toxoid), TT (tetanus toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Marcel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Douglas et al. (1987) J. Bacteriol. 169 (11): 4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP). Another suitable carrier protein is rhizavidin [aa 45-179J-GGGGSSS-SP1500-AAA-SP0785] (CP1).

In a preferred embodiment, the carrier protein of the glycoconjugates is independently selected from the group consisting of TT, DT, DT mutants (such as CRM₁₉₇), H. influenzae protein D, PhtX, PhtD, PhtDE fusions (particularly those described in WO 01/98334 and WO 03/054007), detoxified pneumolysin, PorB, N19 protein, PspA, OMPC, toxin A or B of C. difficile, PsaA, a C₅a peptidase from Streptococcus (SCP) and biotin-strepavidin.

In an embodiment, the carrier protein of the glycoconjugates of the invention is DT (Diphtheria toxoid). In another embodiment, the carrier protein of the glycoconjugates of the invention is TT (tetanus toxid). In an embodiment, the carrier of the glycoproteins is a C5a peptidase from Streptococcus (SCP). In another embodiment, the carrier protein of the glycoconjugates of the invention is PD (H. influenzae protein D; see, e.g., EP0594610 B).

In a preferred embodiment, the capsular saccharides of the invention are conjugated to CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197tox-created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the capsular saccharides of the invention are conjugated to CRM₁₉₇ protein or the A chain of CRM₁₉₇ (see CN103495161). In an embodiment, the capsular saccharides of the invention are conjugated the A chain of CRM₁₉₇ obtained via expression by genetically recombinant E. coli (see CN103495161). In an embodiment, the capsular saccharides of the invention are all conjugated to CRM₁₉₇. In an embodiment, the capsular saccharides of the invention are all conjugated to the A chain of CRM₁₉₇.

Accordingly, in frequent embodiments, the glycoconjugates of the invention comprise CRM₁₉₇ as the carrier protein, wherein the capsular polysaccharide is covalently linked to CRM₁₉₇.

### Capsular Saccharides

The term “saccharide” throughout this specification may indicate polysaccharide or oligosaccharide and includes both. In frequent embodiments, the saccharide is a polysaccharide, in particular a S. pneumoniae capsular polysaccharide.

Capsular polysaccharides are prepared by standard techniques known to those of ordinary skill in the art.

In the present invention, capsular polysaccharides may be prepared or derived, e.g., from serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B of S. pneumoniae. Typically, capsular polysaccharides are produced by growing each S. pneumoniae serotype in a medium (e.g., in a soy-based medium), the polysaccharides are then prepared from the bacteria culture. Bacterial strains of S. pneumoniae used to make the respective polysaccharides that are used in the glycoconjugates of the invention may be obtained from established culture collections or clinical specimens.

In one embodiment, the formulation includes at least 21 different polysaccharides. In one embodiment, the formulation includes at least 22 different polysaccharides. In one embodiment, the formulation includes at least 23 different polysaccharides. In one embodiment, the formulation includes at least 24 different polysaccharides. In one embodiment, the formulation includes at least 25 different polysaccharides. In one embodiment, the formulation includes at least 26 different polysaccharides. In one embodiment, the formulation includes at least 27 different polysaccharides. In one embodiment, the formulation includes at least 28 different polysaccharides. In one embodiment, the formulation includes at least 29 different polysaccharides. In one embodiment, the formulation includes at least 30 different polysaccharides. In one embodiment, the formulation includes at least 31 different polysaccharides. In one embodiment, the formulation includes at least 32 different polysaccharides. In one embodiment, the formulation includes at least 33 different polysaccharides. In one embodiment, the formulation includes at least 34 different polysaccharides. In one embodiment, the formulation includes at least 35 different polysaccharides.

The population of the organism (each S. pneumoniae serotype) is often scaled up from a seed vial to seed bottles and passaged through one or more seed fermentors of increasing volume until production scale fermentation volumes are reached. At the end of the growth cycle the cells are lysed and the lysate broth is then harvested for downstream (purification) processing (see for example WO 2006/110381, WO 2008/118752, and U.S. Patent App. Pub. Nos. 2006/0228380, 2006/0228381, 2008/0102498 and 2008/0286838).

The individual polysaccharides are typically purified through centrifugation, precipitation, ultra-filtration, and/or column chromatography (see for example WO 2006/110352 and WO 2008/118752).

Purified polysaccharides may be activated (e.g., chemically activated) to make them capable of reacting (e.g., with the eTEC spacer) and then incorporated into glycoconjugates of the invention, as further described herein.

S. pneumoniae capsular polysaccharides comprise repeating oligosaccharide units which may contain up to 8 sugar residues.

In an embodiment, capsular saccharide of the invention may be one oligosaccharide unit or a shorter than native length saccharide chain of repeating oligosaccharide units. In an embodiment, capsular saccharide of the invention is one repeating oligosaccharide unit of the relevant serotype.

In an embodiment, capsular saccharide of the invention may be oligosaccharides. Oligosaccharides have a low number of repeat units (typically 5-15 repeat units) and are typically derived synthetically or by hydrolysis of polysaccharides.

Preferably though, all of the capsular saccharides of the present invention and in the vaccine formulations of the present invention are polysaccharides. High molecular weight capsular polysaccharides are able to induce certain antibody immune responses due to the epitopes present on the antigenic surface. The isolation and purification of high molecular weight capsular polysaccharides is preferably contemplated for use in the conjugates, compositions and methods of the present invention.

In some embodiments, the purified polysaccharides before conjugation have a molecular weight of between 10 kDa and 4,000 kDa. In other such embodiments, the polysaccharide has a molecular weight of between 50 kDa and 4,000 kDa. In further such embodiments, the polysaccharide has a molecular weight of between 50 kDa and 3,500 kDa. In other such embodiments, the polysaccharide has a molecular weight of between 50 kDa and 3,000 kDa. In other such embodiments, the polysaccharide has a molecular weight of between 50 kDa and 2,500 kDa. In other such embodiments, the polysaccharide has a molecular weight of between 50 kDa and 2,000 kDa. In other such embodiments, the polysaccharide has a molecular weight of between 50 kDa and 1,750 kDa. In other such embodiments, the polysaccharide has a molecular weight of between 50 kDa and 1,500 kDa. In other such embodiments, the polysaccharide has a molecular weight of between 50 kDa and 1,250 kDa. In other such embodiments, the polysaccharide has a molecular weight of between 50 kDa and 1,000 kDa. In other such embodiments, the polysaccharide has a molecular weight of between 50 kDa and 750 kDa. In other such embodiments, the polysaccharide has a molecular weight of between 50 kDa and 500 kDa. In other such embodiments, the polysaccharide has a molecular weight of between 100 kDa and 4,000 kDa. In other such embodiments, the polysaccharide has a molecular weight of between 100 kDa and 3,500 kDa. In other such embodiments, the polysaccharide has a molecular weight of 100 kDa and 3,000 kDa. In other such embodiments, the polysaccharide has a molecular weight of 100 kDa and 2,500 kDa. In other such embodiments, the polysaccharide has a molecular weight of 100 kDa and 2,000 kDa. In other such embodiments, the polysaccharide has a molecular weight of between 100 kDa and 2,000 kDa. In other such embodiments, the polysaccharide has a molecular weight of between 100 kDa and 1,750 kDa. In other such embodiments, the polysaccharide has a molecular weight of between 100 kDa and 1,500 kDa. In other such embodiments, the polysaccharide has a molecular weight of between 100 kDa and 1,250 kDa. In other such embodiments, the polysaccharide has a molecular weight of between 100 kDa and 1,000 kDa. In other such embodiments, the polysaccharide has a molecular weight of between 100 kDa and 750 kDa. In other such embodiments, the polysaccharide has a molecular weight of between 100 kDa and 500 kDa. In other such embodiments, the polysaccharide has a molecular weight of between 200 kDa and 4,000 kDa. In other such embodiments, the polysaccharide has a molecular weight of between 200 kDa and 3,500 kDa. In other such embodiments, the polysaccharide has a molecular weight of between 200 kDa and 3,000 kDa. In other such embodiments, the polysaccharide has a molecular weight of between 200 kDa and 2,500 kDa. In other such embodiments, the polysaccharide has a molecular weight of between 200 kDa and 2,000 kDa. In other such embodiments, the polysaccharide has a molecular weight of between 200 kDa and 2,000 kDa. In other such embodiments, the polysaccharide has a molecular weight of between 200 kDa and 1,750 kDa. In other such embodiments, the polysaccharide has a molecular weight of between 200 kDa and 1,500 kDa. In other such embodiments, the polysaccharide has a molecular weight of between 200 kDa and 1,250 kDa. In other such embodiments, the polysaccharide has a molecular weight of between 200 kDa and 1,000 kDa. In other such embodiments, the polysaccharide has a molecular weight of between 200 kDa and 750 kDa. In other such embodiments, the polysaccharide has a molecular weight of between 200 kDa and 500 kDa. Any whole number integer within any of the above ranges is contemplated as an embodiment of the disclosure.

A polysaccharide can become slightly reduced in size during normal purification procedures. Additionally, as described herein, polysaccharide can be subjected to sizing techniques before conjugation. Mechanical or chemical sizing maybe employed. Chemical hydrolysis maybe conducted using acetic acid. Mechanical sizing maybe conducted using High Pressure Homogenization Shearing. The molecular weight ranges mentioned above refer to purified polysaccharides before conjugation (e.g., before activation).

In a preferred embodiment the purified polysaccharides, are capsular polysaccharide from serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F or 35B of S. pneumoniae, wherein the capsular polysaccharide has a molecular weight falling within one of the molecular weight ranges as described here above.

As used herein, the term “molecular weight” of polysaccharide or of carrier protein-polysaccharide conjugate refers to molecular weight calculated by size exclusion chromatography (SEC) combined with multiangle laser light scattering detector (MALLS).

In some embodiments, the pneumococcal saccharides from serotypes 9V, 18C, 11A, 15B, 22F and/or 33F of the invention are O-acetylated. In some embodiments, the pneumococcal saccharides from serotypes 9V, 11A, 15B, 22F and/or 33F of the invention are O-acetylated.

The purified polysaccharides described herein are chemically activated to make the saccharides capable of reacting with the carrier protein. These pneumococcal conjugates are prepared by separate processes and formulated into a single dosage formulation as described briefly below and in the art.

Polysaccharides from S. pneumoniae Serotypes 1, 3, 4, 5, 6A, 6B, 7F, 9V, 14, 18C, 19A, 19F and 23F

Capsular saccharides from S. pneumoniae serotypes 1, 3, 4, 5, 6A, 6B, 7F, 9V, 14, 18C, 19A, 19F and 23F may be prepared by standard techniques known to those of ordinary skill in the art (see for example WO 2006/110381). Capsular polysaccharides can be produced by growing each S. pneumoniae serotype in a medium; at the end of the growth cycle the cells are lysed and the lysate broth is then harvested for downstream (purification) processing. The individual polysaccharides are typically purified through centrifugation, precipitation, ultra-filtration, and/or column chromatography (see for example WO 2006/110352 and WO 2008/118752). Purified polysaccharides may be further processed as further described herein to prepare glycoconjugates of the invention.

Polysaccharides from S. pneumoniae Serotype 8

The polysaccharide repeating unit of serotype 8 consists of a linear tetrasaccharide unit with one glucuronic acid (GlcpA), two glucopyranoses (Glcp) and one galactopyranose (Galp) (Jones et al. (1957) The Journal of the American Chemical Society. 79 (11): 2787-2793). All four monosaccharides are linked via 1,4-linkages.

Serotype 8 saccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). In addition, they can be produced using synthetic protocols.

Serotype 8 S. pneumoniae strains may be obtained from established culture collections (such as for example the Streptococcal Reference Laboratory (Centers for Disease Control and Prevention, Atlanta, GA)) or clinical specimens.

Polysaccharides from S. pneumoniae Serotype 10A

The polysaccharide repeating unit of serotype 10A consists of a branched hexasaccharide repeat unit with two galactofuranoses (Gals), three galactopyranoses (Galp), one N-acetylgalactosamine (GalpNAc) and a backbone phosphoribitol (Jones, C. (2005) Carbohydrate Research 269 (1): 175-181). There are two branching monosaccharides at the β-GalpNAc moiety (a β-3-Galp and a β-6-Galf).

Serotype 10A saccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). In addition, they can be produced using synthetic protocols.

Serotype 10A S. pneumoniae strains may be obtained from established culture collections (such as for example the Streptococcal Reference Laboratory (Centers for Disease Control and Prevention, Atlanta, GA)) or clinical specimens.

Polysaccharides from S. pneumoniae Serotype 11A

The polysaccharide repeating unit of serotype 11A consists of a linear tetrasaccharide backbone (two galactopyranoses (Galp) and two glucopyranose (Glcp)) and a pendent phosphoglycerol (Richards et al. (1988) Adv. Exp. Med. Biol. 228:595-597), as shown. The polysaccharide is O-acetylated at multiple locations and, based on the reported data in the literature (Calix et al. (2011) J Bacteriol. 193 (19): 5271-5278), the total amount of O-acetylation in 11A polysaccharide is about 2.6 O-acetyl groups per polysaccharide repeat unit.

Serotype 11A saccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). In addition, they can be produced using synthetic protocols.

Serotype 11A S. pneumoniae strains may be obtained from established culture collections (such as for example the Streptococcal Reference Laboratory (Centers for Disease Control and Prevention, Atlanta, GA)) or clinical specimens.

Polysaccharides from S. pneumoniae Serotype 12F

The polysaccharide repeating unit of serotype 12F consists of a linear trisaccharide backbone (one N-acetylfucosamine (FucₚNAc), one N-acetylgalactosamine (GalpNAc) and one N-acetylmannuronic acid (ManpNAcA)) with two branches: a pendant α-galactopyranose (Galp) linked at C₃ of FucₚNAc and an α-Glcp-(1→2)-α-Glcp disaccharide branch linked at C3 of ManpNAcA (Leontein et al. (1983) Carbohydrate Research 114 (2): 257-266.).

Serotype 12F Streptococcus pneumoniae strains may be obtained from established culture collections (such as for example the Streptococcal Reference Laboratory (Centers for Disease Control and Prevention, Atlanta, GA)) or clinical specimens.

Polysaccharides from S. pneumoniae Serotype 15A

Capsular saccharides from S. pneumoniae serotype 15A may be prepared by standard techniques known to those of ordinary skill in the art (see for example WO 2019/139692). Isolates of pneumococcal serotype 15A can be obtained from the American Type Culture Collection (Manassas). Capsular polysaccharides can be produced by growing each S. pneumoniae serotype in a medium; at the end of the growth cycle the cells are lysed and the lysate broth is then harvested for downstream (purification) processing. The individual polysaccharides are typically purified through centrifugation, precipitation, ultra-filtration, and/or column chromatography (see for example WO 2006/110352 and WO 2008/118752). Purified polysaccharides may be further processed as further described herein to prepare glycoconjugates of the invention.

Polysaccharides from S. pneumoniae Serotype 15B

The polysaccharide repeating unit of serotype 15B consists of a branched trisaccharide backbone (one N-acetylglucosamine (GlcₚNAc), one galactopyranose (Galₚ) and one glucopyranose (Glcp)) with an αGalₚ-βGalₚ disaccharide branch linked to the C₄ hydroxyl group of GlcₚNAc. The phosphoglycerol is linked to the C₃ hydroxyl group of the βGalₚ residue in the disaccharide branch (Jones et al. (2005) Carbohydrate Research 340 (3): 403-409). Capsular polysaccharide from serotype 15C serotype has the identical backbone structure as serotype 15B but lacks the O-acetylation.

Serotype 15B polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). They can also be produced using synthetic protocols known to the man skilled in the art.

Serotype 15B S. pneumoniae strains may be obtained from established culture collections (such as for example the American Type Culture Collection (ATCC, Manassas, VA USA) (e.g., deposit strain No. ATCC10354) or the Streptococcal Reference Laboratory (Centers for Disease Control and Prevention, Atlanta, GA USA)) or from clinical specimens.

Polysaccharides from S. pneumoniae Serotype 22F

The polysaccharide repeating unit of serotype 22F consists of a branched pentasaccharide backbone (one glucuronic acid (GlcₚA), one glucopyranose (Glcₚ), one galactofuranose (Gal_{f}) and two rhamnopyranoses (Rhaₚ)) with a αGlcₚ branch linked to the C₃ hydroxyl group of βRhaₚ (Richards et al. (1989) Canadian Journal of Chemistry 67 (6): 1038-1050). Approximately 80% of the C₂ hydroxyl groups of the βRhaₚ residue in the polysaccharide repeating unit are O-acetylated.

Serotype 22F polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). In addition, they can be produced using

Serotype 22F S. pneumoniae strains may be obtained from established culture collections (such as for example the Streptococcal Reference Laboratory (Centers for Disease Control and Prevention, Atlanta, GA)) or clinical specimens.

Polysaccharides from S. pneumoniae Serotypes 23A and 23B

Capsular saccharides from S. pneumoniae serotypes 23A and 23B may be prepared by standard techniques known to those of ordinary skill in the art (see for example WO 2019/050814). Isolates of pneumococcal serotype 23A can be obtained from the Merck Culture Collection and for serotype 23B from Centers for Disease Control and Prevention (Atlanta, GA). Capsular polysaccharides can be produced by growing each S. pneumoniae serotype in a medium; at the end of the growth cycle the cells are lysed and the lysate broth is then harvested for downstream (purification) processing. The individual polysaccharides are typically purified through centrifugation, precipitation, ultra-filtration, and/or column chromatography (see for example WO 2006/110352 and WO 2008/118752). Purified polysaccharides may be further processed as further described herein to prepare glycoconjugates of the invention.

Polysaccharides from S. pneumoniae Serotype 24F

Capsular saccharides from S. pneumoniae serotype 24F may be prepared by standard techniques known to those of ordinary skill in the art (see for example WO 2019/050815). Isolates of pneumococcal serotype 24F can be obtained from the Merck Culture Collection. Capsular polysaccharides can be produced by growing each S. pneumoniae serotype in a medium; at the end of the growth cycle the cells are lysed and the lysate broth is then harvested for downstream (purification) processing. The individual polysaccharides are typically purified through centrifugation, precipitation, ultra-filtration, and/or column chromatography (see for example WO 2006/110352 and WO 2008/118752). Purified polysaccharides may be further processed as further described herein to prepare glycoconjugates of the invention.

Polysaccharides from S. pneumoniae Serotype 33F

The polysaccharide repeating unit of serotype 33F consists of a branched pentasaccharide backbone (two galactopyranoses (Galₚ), two galactofuranoses (Gal_{f}) and one glucopyranose (Glcₚ) with a terminal αGalₚ linked to the C₂ hydroxyl group of αGalₚ residue within the backbone (Lemercinier et al. (2006) Carbohydrate Research 341 (1): 68-74.). It has been reported in the literature that the C₂ hydroxyl group of the backbone 3-β-Gal residue is O-acetylated.

Serotype 33F polysaccharides can be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). In addition, they can be produced using

Serotype 33F S. pneumoniae strains may be obtained from established culture collections (such as for example the Streptococcal Reference Laboratory (Centers for Disease Control and Prevention, Atlanta, GA)) or clinical specimens.

Polysaccharides from S. pneumoniae Serotype 35B

Capsular saccharides from S. pneumoniae serotype 35B may be prepared by standard techniques known to those of ordinary skill in the art (see for example WO 2020/247299). Isolates of pneumococcal serotype 35B can be obtained from the Merck Culture Collection. Capsular polysaccharides can be produced by growing each S. pneumoniae serotype in a medium; at the end of the growth cycle the cells are lysed and the lysate broth is then harvested for downstream (purification) processing. The individual polysaccharides are typically purified through centrifugation, precipitation, ultra-filtration, and/or column chromatography (see for example WO 2006/110352 and WO 2008/118752). Purified polysaccharides may be further processed as further described herein to prepare glycoconjugates of the invention.

### Glycoconjugates

The purified saccharides are chemically activated to make the saccharides (i.e., activated saccharides) capable of reacting with the carrier protein. Once activated, each capsular saccharide is separately conjugated to a carrier protein to form a glycoconjugate.

In the present invention, gycoconjugates may be prepared or derived, e.g., from serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B of S. pneumoniae.

In one embodiment, the formulation includes at least 21 different glycoconjugates. In one embodiment, the formulation includes at least 22 different glycoconjugates. In one embodiment, the formulation includes at least 23 different glycoconjugates. In one embodiment, the formulation includes at least 24 different glycoconjugates. In one embodiment, the formulation includes at least 25 different glycoconjugates. In one embodiment, the formulation includes at least 26 different glycoconjugates. In one embodiment, the formulation includes at least 27 different glycoconjugates. In one embodiment, the formulation includes at least 28 different glycoconjugates. In one embodiment, the formulation includes at least 29 different glycoconjugates. In one embodiment, the formulation includes at least 30 different glycoconjugates. In one embodiment, the formulation includes at least 31 different glycoconjugates. In one embodiment, the formulation includes at least 32 different the formulation includes at least 33 different glycoconjugates. In one embodiment, glycoconjugates. In one embodiment, the formulation includes at least 34 different glycoconjugates. In one embodiment, the formulation includes at least 35 different glycoconjugates.

In one embodiment, each capsular saccharide is conjugated to the same carrier protein. The chemical activation of the saccharides and subsequent conjugation to the carrier protein can be achieved by the activation and conjugation methods known in the art and briefly described below.

Glycoconjugates from S. pneumoniae Serotype 1, 3, 4, 5, 6A, 6B, 7F, 9V, 14, 18C, 19A, 19F and 23F

Capsular polysaccharides from serotypes 1, 3, 4, 5, 6A, 6B, 7F, 9V, 14, 18C, 19A, 19F and 23F of S. pneumoniae are prepared by standard techniques known to those of ordinary skill in the art (see for example WO 2006/110381, WO 2008/118752, WO 2006/110352, and U.S. Patent App. Pub. Nos. 2006/0228380, 2006/0228381, 2008/0102498 and 2008/0286838).

In a preferred embodiment, at least one of capsular polysaccharides from serotypes 1, 3, 4, 5, 6A, 6B, 7F, 9V, 14, 18C, 19A, 19F and 23F of S. pneumoniae is conjugated to the carrier protein by reductive amination (such as described in U.S. Patent Appl. Pub. Nos. 2006/0228380, 2007/0231340, 2007/0184071 and 2007/0184072, WO 2006/110381, WO 2008/079653, and WO 2008/143709). In a preferred embodiment, the capsular polysaccharides from serotypes 1, 3, 4, 5, 6A, 6B, 7F, 9V, 14, 18C, 19A, 19F and 23F of S. pneumoniae are all conjugated to the carrier protein by reductive amination.

Glycoconjugates from S. pneumoniae Serotype 8, 11A, 15 B and 22F

In an embodiment, the serotype 8, 11A, 15 B and 22F glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO 93/15760, WO 95/08348 and WO 96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In preferred embodiments, the serotype 8, 11A, 15 B and 22F glycoconjugates of the invention are prepared using reductive amination. Reductive amination involves two steps, (1) oxidation of the polysaccharide to generate aldehyde functionalities from vicinal diols in individual hexasaccharide unit, (2) reduction of the activated polysaccharide and a carrier protein (e.g., CRM₁₉₇) to form a conjugate. Methods of preparing glycoconjugates from serotypes 8, 11A, 15 B and 22F S. pneumoniae are known and are described in WO2015110941.

Glycoconjugates from S. pneumoniae Serotype 12F

In the glycoconjugates from S. pneumoniae serotype 12F of the present invention, the saccharide is selected from the group consisting of a polysaccharide and an oligosaccharide, and the carrier protein is selected from any suitable carrier as described herein or known to those of skill in the art. In some preferred embodiments, the saccharide is a polysaccharide from serotype 12F S. pneumoniae.

In an embodiment, glycoconjugates from S. pneumoniae serotype 12F are prepared using CDAP. The polysaccharides are activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier.

Other techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In an embodiment, capsular polysaccharides from serotypes 12F S. pneumoniae are conjugated to the carrier protein by reductive amination. Reductive amination involves two steps, (1) oxidation of the polysaccharide to generate aldehyde functionalities from vicinal diols in individual hexasaccharide unit, (2) reduction of the activated polysaccharide and a carrier protein to form a conjugate. Methods of preparing glycoconjugates from serotypes 12F S. pneumoniae are known and are described in WO2015110941.

Glycoconjugates from S. pneumoniae Serotype 15A

Capsular polysaccharides from serotype 15A of S. pneumoniae are prepared by standard techniques known to those of ordinary skill in the art (see for example WO 2019/139692).

In preferred embodiments, the serotype 15A glycoconjugates of the invention are prepared using reductive amination. Reductive amination involves two steps, (1) oxidation of the polysaccharide to generate aldehyde functionalities from vicinal diols in individual hexasaccharide unit, (2) reduction of the activated polysaccharide and a carrier protein (e.g., CRM₁₉₇) to form a conjugate. Methods of preparing glycoconjugates from serotype 15A S. pneumoniae are known and are described in WO 2019/139692.

Glycoconjugates from S. pneumoniae Serotypes 23A and 23B

Capsular polysaccharides from serotypes 23A and 23B of S. pneumoniae are prepared by standard techniques known to those of ordinary skill in the art (see for example WO 2019/050814).

In preferred embodiments, the serotypes 23A and 23B glycoconjugates of the invention are prepared using reductive amination. Reductive amination involves two steps, (1) oxidation of the polysaccharide to generate aldehyde functionalities from vicinal diols in individual hexasaccharide unit, (2) reduction of the activated polysaccharide and a carrier protein (e.g., CRM₁₉₇) to form a conjugate. Methods of preparing glycoconjugates from serotypes 23A, 23B and 24F S. pneumoniae are known and are described in WO 2019/050814.

Glycoconjugates from S. pneumoniae Serotype 24F

Capsular polysaccharides from serotype 24F of S. pneumoniae are prepared by standard techniques known to those of ordinary skill in the art (see for example WO 2019/050815).

In preferred embodiments, the serotype 24F glycoconjugates of the invention are prepared using reductive amination. Reductive amination involves two steps, (1) oxidation of the polysaccharide to generate aldehyde functionalities from vicinal diols in individual hexasaccharide unit, (2) reduction of the activated polysaccharide and a carrier protein (e.g., CRM₁₉₇) to form a conjugate. Methods of preparing glycoconjugates from serotype 24F S. pneumoniae are known and are described in WO 2019/050815.

Glycoconjugates from S. pneumoniae Serotype 33F

In an embodiment, the serotype 33F glycoconjugates are obtained by activating polysaccharide with 1-cyano-4-dimethylamino pyridinium tetrafluoroborate (CDAP) to form a cyanate ester. The activated polysaccharide may be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide, SIB, SIAB, sulfo-SIAB, SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO 93/15760, WO 95/08348 and WO 96/129094.

Other suitable techniques use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In certain embodiments, the serotype 33F glycoconjugates of the invention are prepared using reductive amination. In such embodiment, the serotype 33F glycoconjugates of the invention maybe prepared using reductive amination in aqueous phase (RAC/aqueous). Reductive amination in aqueous phase has been successfully applied to produce pneumococcal conjugate vaccine (see, e.g., WO 2006/110381). Preferably though, when using reductive amination, the serotype 33F glycoconjugates are prepared via reductive amination in DMSO (RAC/DMSO). In view of the challenges associated with the preservation of O-acetyl functionality using RAC/aqueous process, reductive amination in DMSO is preferred. RAC/DMSO has been successfully applied to produce pneumococcal conjugate vaccine (see, e.g., WO 2006/110381).

In preferred embodiments, the serotype 33F glycoconjugates of the invention are prepared using eTEC conjugation (hereinafter “serotype 33F eTEC linked glycoconjugates”), such as described at Examples 1, 2 and 3 and in WO 2014/027302.

Glycoconjugates from S. pneumoniae Serotype 35B

Capsular polysaccharides from serotype 35B of S. pneumoniae are prepared by standard techniques known to those of ordinary skill in the art (see for example WO 2020/247299).

In preferred embodiments, the serotype 35B glycoconjugates of the invention are prepared using reductive amination. Reductive amination involves two steps, (1) oxidation of the polysaccharide to generate aldehyde functionalities from vicinal diols in individual hexasaccharide unit, (2) reduction of the activated polysaccharide and a carrier protein (e.g., CRM₁₉₇) to form a conjugate. Methods of preparing glycoconjugates from serotype 35B S. pneumoniae are known and are described in WO 2020/247299.

### Combinations of Glycoconjugates

In an embodiment the vaccine formulations of the invention comprises any of the glycoconjugates or combination of glycoconjugates disclosed herein.

In one embodiment, the formulations include at least 25 glycoconjugates. In one embodiment, the formulation includes at least 21 glycoconjugates. In one embodiment, the formulation includes at least 22 glycoconjugates. In one embodiment, the formulation includes at least 23 glycoconjugates. In one embodiment, the formulation includes at least 24 glycoconjugates. In one embodiment, the formulation includes at least 25 glycoconjugates. In one embodiment, the formulation includes at least 26 glycoconjugates. In one embodiment, the formulation includes at least 27 glycoconjugates. In one embodiment, the formulation includes at least 28 glycoconjugates. In one embodiment, the formulation includes at least 29 glycoconjugates. In one embodiment, the formulation includes at least 30 glycoconjugates. In one embodiment, the formulation includes at least 31 glycoconjugates. In one embodiment, the formulation includes at least 32 glycoconjugates. In one embodiment, the formulation includes at least 33 glycoconjugates. In one embodiment, the formulation includes at least 34 glycoconjugates. In one embodiment, the formulation includes at least 35 glycoconjugates.

In an embodiment, the formulations of the present invention include at least glycoconjugates derived from S. pneumoniae serotypes 4, 6B, 9V, 14, 18C, 19F and 23F.

In an embodiment, the formulations of the present invention include at least glycoconjugates derived from S. pneumoniae serotypes 1, 4, 5, 6B, 7F, 9V, 14, 18C, 19F and 23F.

In an embodiment, formulations of the present invention include at least glycoconjugates derived from S. pneumoniae serotypes 1, 3, 4, 5, 6A, 6B, 7F, 9V, 14, 18C, 19A, 19F, 23F and 33F.

In an embodiment, the formulations of the present invention include at least glycoconjugates derived from S. pneumoniae serotypes 1, 3, 4, 5, 6A, 6B, 7F, 9V, 14, 18C, 19A, 19F, 22F, 23F and 33F.

In an embodiment, formulations of the present invention include at least glycoconjugates derived from S. pneumoniae serotypes 1, 3, 4, 5, 6A, 6B, 7F, 9V, 14, 18C, 19A, 19F, 22F, 23F and 33F.

In an embodiment, formulations of the present invention include at least glycoconjugates derived from S. pneumoniae serotypes 1, 3, 5, 4, 6A, 6B, 7F, 8, 9V, 10A, 11A, 12F, 14, 15B, 18C, 19A, 19F, 22F, 23F and 33F.

In an embodiment, formulations of the present invention include at least glycoconjugates derived from S. pneumoniae serotypes 1, 2, 3, 4, 5, 6B, 7F, 8, 9V, 9N, 10A, 11A, 12F, 14, 15B, 17F, 18C, 19A, 19F, 20, 22F, 23F and 33F.

In an embodiment, formulations of the present invention include at least glycoconjugates derived from S. pneumoniae serotypes 1, 2, 3, 4, 5, 6A, 6B, 7F, 8, 9V, 9N, 10A, 11A, 12F, 14, 15B, 17F, 18C, 19A, 19F, 20, 22F, 22F and 33F.

In an embodiment, formulations of the present invention include at least glycoconjugates derived from S. pneumoniae serotypes 1, 2, 3, 4, 5, 6A, 6B, 7F, 8, 9N, 9V, 10A, 11A, 12F, 14, 15A, 15B, 18C, 19A, 19F, 22F, 23A, 23B, 24F, 33F and 35B.

In an embodiment, formulations of the present invention include at least glycoconjugates derived from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F.

In an embodiment, formulations of the present invention include at least glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

In an embodiment, the formulations of the present invention include at least one glycoconjugate derived from S. pneumoniae serotype 1, a glycoconjugate of S. pneumoniae serotype 3, a glycoconjugate of S. pneumoniae serotype 4, a glycoconjugate of S. pneumoniae serotype 5, a glycoconjugate of S. pneumoniae serotype 6A, a glycoconjugate of S. pneumoniae serotype 6B, a glycoconjugate of S. pneumoniae serotype 7F, a glycoconjugate of S. pneumoniae serotype 8, a glycoconjugate of S. pneumoniae serotype 9V, a glycoconjugate of S. pneumoniae serotype 10A, a glycoconjugate of S. pneumoniae serotype 11A, a glycoconjugate of S. pneumoniae serotype 12F, a glycoconjugate of S. pneumoniae serotype 14, a glycoconjugate of S. pneumoniae serotype 15A, a glycoconjugate of S. pneumoniae serotype 15B, a glycoconjugate of S. pneumoniae serotype 18C, a glycoconjugate of S. pneumoniae serotype 19A, a glycoconjugate of S. pneumoniae serotype 19F, a glycoconjugate of S. pneumoniae serotype 22F, a glycoconjugate of S. pneumoniae serotype 23A, a glycoconjugate of S. pneumoniae serotype 23B, a glycoconjugate of S. pneumoniae serotype 23F, a glycoconjugate of S. pneumoniae serotype 24F, a glycoconjugate of S. pneumoniae serotype 33F, a glycoconjugate of S. pneumoniae serotype 35B and combinations thereof.

In an embodiment, the formulations include glycoconjugates derived from S. pneumoniae serotype 1, a glycoconjugate of S. pneumoniae serotype 3, a glycoconjugate of S. pneumoniae serotype 4, a glycoconjugate of S. pneumoniae serotype 5, a glycoconjugate of S. pneumoniae serotype 6A, a glycoconjugate of S. pneumoniae serotype 6B, a glycoconjugate of S. pneumoniae serotype 7F, a glycoconjugate of S. pneumoniae serotype 8, a glycoconjugate of S. pneumoniae serotype 9V, a glycoconjugate of S. pneumoniae serotype 10A, a glycoconjugate of S. pneumoniae serotype 11A, a glycoconjugate of S. pneumoniae serotype 12F, a glycoconjugate of S. pneumoniae serotype 14, a glycoconjugate of S. pneumoniae serotype 15A, a glycoconjugate of S. pneumoniae serotype 15B, a glycoconjugate of S. pneumoniae serotype 18C, a glycoconjugate of S. pneumoniae serotype 19A, a glycoconjugate of S. pneumoniae serotype 19F, a glycoconjugate of S. pneumoniae serotype 22F, a glycoconjugate of S. pneumoniae serotype 23A, a glycoconjugate of S. pneumoniae serotype 23B, a glycoconjugate of S. pneumoniae serotype 23F, a glycoconjugate of S. pneumoniae serotype 24F, a glycoconjugate of S. pneumoniae serotype 33F and a glycoconjugate of S. pneumoniae serotype 35B.

In an embodiment, the formulations of the present invention include at least glycoconjugates derived from S. pneumoniae serotypes 4, 6B, 9V, 14, 18C, 19F and 23F and the glycoconjugates are conjugated to CRM₁₉₇.

In an embodiment, formulations of the present invention include at least glycoconjugates derived from S. pneumoniae serotypes 1, 4, 5, 6B, 7F, 9V, 14, 18C, 19F and 23F. In an embodiment, the glycoconjugates of S. pneumoniae serotypes 1, 4, 5, 7F, 9V and/or 23F are conjugated to PD, the glycoconjugates of S. pneumoniae serotype 18C are conjugated to TT and the glycoconjugates of S. pneumoniae serotype 19F are conjugated to DT.

In an embodiment, formulations of the present invention include at least glycoconjugates derived from S. pneumoniae serotypes 1, 3, 4, 5, 6A, 6B, 7F, 9V, 14, 18C, 19A, 19F. 23F and 33F and the glycoconjugates of the S. pneumoniae serotypes are conjugated to CRM₁₉₇.

In an embodiment, formulations of the present invention include at least glycoconjugates derived from S. pneumoniae serotypes 1, 3, 4, 5, 6A, 6B, 7F, 9V, 14, 18C, 19A, 19F, 22F, 23F and 33F and the glycoconjugates of the S. pneumoniae serotypes are conjugated to CRM_{197.}

In an embodiment, formulations of the present invention include at least glycoconjugates derived from S. pneumoniae serotypes 1, 3, 4, 5, 6A, 6B, 7F, 9V, 14, 18C, 19A, 19F, 22F, 23F and 33F and the glycoconjugates of S. pneumoniae serotypes are conjugated to CRM₁₉₇.

In an embodiment, formulations of the present invention include at least glycoconjugates derived from S. pneumoniae serotypes 1, 2, 3, 4, 5, 6B, 7F, 8, 9V, 9N, 10A, 11A, 12F, 14, 15B, 17F, 18C, 19A, 19F, 20, 22F, 23F and 33F and the glycoconjugates of the S. pneumoniae serotypes are conjugated to CRM₁₉₇.

In an embodiment, formulations of the present invention include at least glycoconjugates derived from S. pneumoniae serotypes 1, 2, 3, 4, 5, 6A, 6B, 7F, 8, 9V, 9N, 10A, 11A, 12F, 14, 15B, 17F, 18C, 19A, 19F, 20, 22F, 22F and 33F. In an embodiment, the glycoconjugates of the S. pneumoniae serotypes are conjugated to CRM₁₉₇. In an embodiment, the glycoconjugates of the S. pneumoniae serotypes 1, 2, 5, 6A, 7F, 8, 9N, 10A, 11A, 12F, 15A, 15B, 19A, 22F, 23A, 23B, 24F, 33F and 35B are conjugated to CRM₁₉₇ and the glycoconjugates of the S. pneumoniae serotype 3 are conjugated to SCP.

In an embodiment, formulations of the present invention include at least glycoconjugates derived from S. pneumoniae serotypes 1, 2, 3, 4, 5, 6A, 6B, 7F, 8, 9V, 10A, 11A, 12F, 14, 15A, 15B, 18C, 19A, 19F, 22F, 23A, 23B, 23B, 24F, 33F and 35B. In an embodiment, the glycoconjugates of the S. pneumoniae serotypes are conjugated to CRM₁₉₇. In an embodiment, the glycoconjugates of the S. pneumoniae serotypes 1, 2, 4, 5, 6A, 6B, 7F, 8, 9V, 10A, 11A, 12F, 14, 15A, 15B, 18C, 19A, 19F, 22F, 23A, 23B, 23B, 24F, 33F and 35B are conjugated to CRM₁₉₇ and the glycoconjugates of the S. pneumoniae serotype 3 are conjugated to SCP.

In an embodiment, formulations of the present invention include at least glycoconjugates derived from S. pneumoniae serotypes 1, 2, 3, 4, 5, 6A, 6B, 7F, 8, 9N, 9V, 10A, 11A, 12F, 14, 15A, 15B, 18C, 19A, 19F, 22F, 23A, 23B, 24F, 33F and 35B. In an embodiment, at least two of the glycoconjugates of S. pneumoniae serotypes are conjugated to TT. In one embodiment, the at least two the glycoconjugates of S. pneumoniae serotypes conjugated to TT are selected from S. pneumoniae serotypes 1, 3, 5, 15B and 22F. In an embodiment, at least 17 of the glycoconjugates of S. pneumoniae serotypes are conjugated to CRM₁₉₇. In an embodiment, the at least 17 the glycoconjugates of S. pneumoniae serotypes conjugated to CRM₁₉₇ are selected from S. pneumoniae serotypes 1, 3, 4, 5, 6A, 6B, 7F, 8, 9N, 9V, 10A, 11A, 12F, 14, 15B, 18C, 19A, 19F 22F, 23F and 33F.

In an embodiment, formulations of the present invention include at least glycoconjugates derived from S. pneumoniae serotypes 1, 3, 4, 5, 6A, 6B, 7F, 8, 9N, 9V, 10A, 11A, 12F, 14, 15B, 18C, 19A, 19F, 22F, 23F and 33F. In an embodiment, the glycoconjugates of S. pneumoniae serotypes 1, 4, 5, 7F, 9V and/or 23F are conjugated to PD, glycoconjugates of S. pneumoniae serotype 18C are conjugated to TT and glycoconjugates of S. pneumoniae serotype 19F are conjugated to DT.

In one embodiment formulations of the present invention include at least glycoconjugates derived from S. pneumoniae serotype 1 conjugated to CRM_{197,} S. pneumoniae serotype 3 conjugated to CRM_{197,} S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.

Preferably, all the glycoconjugates of the above vaccine formulations are individually conjugated to the carrier protein.

### Dosing

The amount of glycoconjugate(s) in each dose is selected as an amount which induces an immunoprotective response without significant, adverse side effects in typical vaccinees. Such amount will vary depending upon which specific immunogen is employed and how it is presented.

### Glycoconjugate Amount

The amount of a particular glycoconjugate in a vaccine formulation can be calculated based on total polysaccharide for that conjugate (conjugated and non-conjugated). For example, a glycoconjugate with 20% free polysaccharide will have about 80 μg of conjugated polysaccharide and about 20 μg of nonconjugated polysaccharide in a 100 μg polysaccharide dose. The amount of glycoconjugate can vary depending upon the pneumococcal serotype. The saccharide concentration can be determined by the uronic acid assay.

The “immunogenic amount” of the different polysaccharide components in the vaccine formulations, may diverge and each may comprise about 1 μg, about 2 μg, about 3 μg, about 4 μg, about 5 μg, about 6 μg, about 7 μg, about 8 μg, about 9 μg, about 10 μg, about 15 μg, about 20 μg, about 30 μg, about 40 μg, about 50 μg, about 60 μg, about 70 μg, about 80 μg, about 90 μg, or about 100 μg of any particular polysaccharide antigen.

Generally, each dose will comprise 0.1 μg to 100 μg of polysaccharide for a given serotype, particularly 0.5 μg to 20 μg, more particularity 1.0 μg to 10 μg, and even more particularly 2.0 μg to 5.0 μg. Any whole number integer within any of the above ranges is contemplated as an embodiment of the disclosure.

In an embodiment, each dose will comprise about 1.0 μg to about 6.0 μg polysaccharide for each particular glycoconjugate. In an embodiment, each dose will comprise about 1.5 μg to about 5.0 μg polysaccharide for each particular glycoconjugate. In a preferred embodiment, each dose will comprise about 2.0 μg to about 4.0 μg polysaccharide for each particular glycoconjugate. In a more preferred embodiment, each dose will comprise about 2.0 μg to about 3.0 μg polysaccharide for each particular glycoconjugate In an embodiment, each dose will comprise about 1.0 μg of polysaccharide for each particular glycoconjugate. In an embodiment, each dose will comprise about 1.2 μg of polysaccharide for each particular glycoconjugate. In an embodiment, each dose will comprise about 1.4 μg of polysaccharide for each particular glycoconjugate. In an embodiment, each dose will comprise about 1.6 μg of polysaccharide for each particular glycoconjugate. In an embodiment, each dose will comprise about 1.8 μg of polysaccharide for each particular glycoconjugate. In an embodiment, each dose will comprise about 2.0 μg of polysaccharide for each particular glycoconjugate. In an embodiment, each dose will comprise about 2.2 μg of polysaccharide for each particular glycoconjugate. In an embodiment, each dose will comprise about 2.4 μg of polysaccharide for each particular glycoconjugate. In an embodiment, each dose will comprise about 2.6 μg of polysaccharide for each particular glycoconjugate. In an embodiment, each dose will comprise about 2.8 μg of polysaccharide for each particular glycoconjugate. In an embodiment, each dose will comprise about 3.0 μg of polysaccharide for each particular glycoconjugate. In an embodiment, each dose will comprise about 3.2 μg of polysaccharide for each particular glycoconjugate. In an embodiment, each dose will comprise about 3.4 μg of polysaccharide for each particular glycoconjugate. In an embodiment, each dose will comprise about 3.6 μg of polysaccharide for each particular glycoconjugate. In an embodiment, each dose will comprise about 3.8 μg of polysaccharide for each particular glycoconjugate. In an embodiment, each dose will comprise about 4.0 μg of polysaccharide for each particular glycoconjugate. In an embodiment, each dose will comprise about 4.2 μg of polysaccharide for each particular glycoconjugate. In an embodiment, each dose will comprise about 4.4 μg of polysaccharide for each particular glycoconjugate. In an embodiment, each dose will comprise about 4.6 μg of polysaccharide for each particular glycoconjugate. In an embodiment, each dose will comprise about 4.8 μg of polysaccharide for each particular glycoconjugate. In an embodiment, each dose will comprise about 5.0 μg of polysaccharide for each particular glycoconjugate. In an embodiment, each dose will comprise about 5.2 μg of polysaccharide for each particular glycoconjugate. In an embodiment, each dose will comprise about 5.4 μg of polysaccharide for each particular glycoconjugate. In an embodiment, each dose will comprise about 5.6 μg of polysaccharide for each particular glycoconjugate. In an embodiment, each dose will comprise about 5.8 μg of polysaccharide for each particular glycoconjugate. In an embodiment, each dose will comprise about 6.0 μg of polysaccharide for each particular glycoconjugate.

In an embodiment, each dose will comprise about 1.0 μg to about 3.0 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.5 μg to about 3.0 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In a preferred embodiment, each dose will comprise about 2.0 μg to about 3.0 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In a more preferred embodiment, each dose will comprise about 2.5 μg to about 3.0 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.0 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B In an embodiment, each dose will comprise about 1.1 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.2 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.3 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.4 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.5 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.6 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.7 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.8 of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 1.9 of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.0 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.1 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.2 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.3 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.4 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.5 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.6 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.7 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.8 of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 2.9 of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B. In an embodiment, each dose will comprise about 3.0 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B.

### Carrier Amount

Generally, each dose will comprise 10 μg to 150 μg of carrier protein, particularly 15 μg to 100 μg of carrier protein, more particularly 25 μg to 75 μg of carrier protein, and even more particularly 50 μg to 70 μg of carrier protein. In an embodiment, said carrier protein is CRM₁₉₇. In an embodiment, said carrier protein is SCP.

In an embodiment, each dose will comprise about 25 μg of carrier protein. In an embodiment, each dose will comprise about 26 μg of carrier protein. In an embodiment, each dose will comprise about 27 μg of carrier protein. In an embodiment, each dose will comprise about 28 μg of carrier protein. In an embodiment, each dose will comprise about 29 μg of carrier protein. In an embodiment, each dose will comprise about 30 μg of carrier protein. In an embodiment, each dose will comprise about 31 μg of carrier protein. In an embodiment, each dose will comprise about 32 μg of carrier protein. In an embodiment, each dose will comprise about 33 μg of carrier protein. In an embodiment, each dose will comprise about 34 μg of carrier protein. In an embodiment, each dose will comprise about 35 μg of carrier protein. In an embodiment, each dose will comprise about 36 μg of carrier protein. In an embodiment, each dose will comprise about 37 μg of carrier protein. In an embodiment, each dose will comprise about 38 μg of carrier protein. In an embodiment, each dose will comprise about 39 μg of carrier protein. In an embodiment, each dose will comprise about 40 μg of carrier protein. In an embodiment, each dose will comprise about 41 μg of carrier protein. In an embodiment, each dose will comprise about 42 μg of carrier protein. In an embodiment, each dose will comprise about 43 μg of carrier protein. In an embodiment, each dose will comprise about 44 μg of carrier protein. In an embodiment, each dose will comprise about 45 μg of carrier protein. In an embodiment, each dose will comprise about 46 μg of carrier protein. In an embodiment, each dose will comprise about 47 μg of carrier protein. In an embodiment, each dose will comprise about 48 μg of carrier protein. In an embodiment, each dose will comprise about 49 μg of carrier protein. In an embodiment, each dose will comprise about 50 μg of carrier protein. In an embodiment, each dose will comprise about 51 μg of carrier protein. In an embodiment, each dose will comprise about 52 μg of carrier protein. In an embodiment, each dose will comprise about 53 μg of carrier protein. In an embodiment, each dose will comprise about 54 μg of carrier protein. In an embodiment, each dose will comprise about 55 μg of carrier protein. In an embodiment, each dose will comprise about 56 μg of carrier protein. In an embodiment, each dose will comprise about 57 μg of carrier protein. In an embodiment, each dose will comprise about 58 μg of carrier protein. In an embodiment, each dose will comprise about 59 μg of carrier protein. In an embodiment, each dose will comprise about 60 μg of carrier protein. In an embodiment, each dose will comprise about 61 μg of carrier protein. In an embodiment, each dose will comprise about 62 μg of carrier protein. In an embodiment, each dose will comprise about 63 μg of carrier protein. In an embodiment, each dose will comprise about 64 μg of carrier protein. In an embodiment, each dose will comprise about 65 μg of carrier protein. In an embodiment, each dose will comprise about 66 μg of carrier protein. In an embodiment, each dose will comprise about 67 μg of carrier protein. In an embodiment, each dose will comprise about 68 μg of carrier protein. In an embodiment, each dose will comprise about 69 μg of carrier protein. In an embodiment, each dose will comprise about 70 μg of carrier protein. In an embodiment, each dose will comprise about 71 μg of carrier protein. In an embodiment, each dose will comprise about 72 μg of carrier protein. In an embodiment, each dose will comprise about 73 μg of carrier protein. In an embodiment, each dose will comprise about 74 μg of carrier protein. In an embodiment, each dose will comprise about 75 μg of carrier protein.

In an embodiment, each dose will comprise between about 60 μg and 70 μg of carrier protein.

### Further Antigens

In some embodiments, vaccine formulations disclosed herein comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more antigens. In some embodiments, the vaccine formulation comprises more than one antigen specific for a particular viral or bacterial species. In particular embodiments, the vaccine formulation comprises more than one antigen specific for S. pneumoniae. In other embodiments, the vaccine formulation comprises antigens specific for a combination of two or more bacterial species. In still other embodiments, the vaccine formulation comprises antigens specific for a combination of two or more viral species. In some embodiments, the vaccine formulation comprises antigens specific for a combination of at least one viral species and at least one bacterial species.

In some embodiments, the antigens selected are specific for chickenpox or shingles, human respiratory syncytial virus infection (RSV), Cytomegalovirus infection (CMV), Human metapneumovirus, Human parainfluenza viruses type 1 or type 3, Lyme disease, Streptococcus pneumonia, Clostridioides difficile, Coronaviruses, Escherichia coli, Klebsiella pneumoniae, influenza, HIV-1, Hepatitis A, Hepatitis B, Human Papilloma virus, Meningococcal type A meningitis, Meningococcal type B meningitis, Meningococcal type C meningitis, Meningococcal type W meningitis, Meningococcal type Y meningitis, Tetanus, Diphtheria, Pertussis, Polio, Haemophilus influenza type B, Dengue, Hand Foot and Mouth Disease, Typhoid, Pneumococcus, Japanese encephalitis virus, Anthrax, Shingles, Malaria, Norovirus, or cancer.

Vaccine formulations of the invention comprise conjugated S. pneumoniae saccharide antigens (glycoconjugates). They may also further include antigens from other pathogens, particularly from bacteria and/or viruses. Preferred further antigens are selected from: a diphtheria toxoid (D), a tetanus toxoid (T), a pertussis antigen (P), which is typically acellular (Pa), a hepatitis B virus (HBV) surface antigen (HBsAg), a hepatitis A virus (HAV) antigen, a conjugated Haemophilus influenzae type b capsular saccharide (Hib), inactivated poliovirus vaccine (IPV).

In an embodiment, the vaccine formulations of the invention comprise D-T-Pa. In an embodiment, the vaccine formulations of the invention comprise D-T-Pa-Hib, D-T-Pa-IPV or D-T-Pa-HBsAg. In an embodiment, the vaccine formulations of the invention comprise D-T-Pa-HBsAg-IPV or D-T-Pa-HBsAg-Hib. In an embodiment, the vaccine formulations of the invention comprise D-T-Pa-HBsAg-IPV-Hib.

Pertussis antigens: Bordetella pertussis causes whooping cough. Pertussis antigens in vaccines are either cellular (whole cell, in the form of inactivated B. pertussis cells) or acellular. Preparation of cellular pertussis antigens is well documented (e.g., it may be obtained by heat inactivation of phase I culture of B. pertussis). Preferably, however, the invention uses acellular antigens. Where acellular antigens are used, it is preferred to use one, two or (preferably) three of the following antigens: (1) detoxified pertussis toxin (pertussis toxoid, or PT); (2) filamentous hemagglutinin (FHA); (3) pertactin (also known as the 69 kiloDalton outer membrane protein). FHA and pertactin may be treated with formaldehyde prior to use according to the invention. PT is preferably detoxified by treatment with formaldehyde and/or glutaraldehyde. Acellular pertussis antigens are preferably adsorbed onto one or more aluminum salt adjuvants. As an alternative, they may be added in an unadsorbed state. Where pertactin is added then it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is most preferred.

Inactivated poliovirus vaccine: Poliovirus causes poliomyelitis. Rather than use oral poliovirus vaccine, preferred embodiments of the invention use IPV. Prior to administration to patients, polioviruses must be inactivated, and this can be achieved by treatment with formaldehyde. Poliomyelitis can be caused by one of three types of poliovirus. The three types are similar and cause identical symptoms, but they are antigenically different and infection by one type does not protect against infection by others. It is therefore preferred to use three poliovirus antigens in the invention: poliovirus Type 1 (e.g., Mahoney strain), poliovirus Type 2 (e.g., MEF-1 strain), and poliovirus Type 3 (e.g., Saukett strain). The viruses are preferably grown, purified and inactivated individually, and are then combined to give a bulk trivalent mixture for use with the invention.

Diphtheria toxoid: Corynebacterium diphtheriae causes diphtheria. Diphtheria toxin can be treated (e.g., using formalin or formaldehyde) to remove toxicity while retaining the ability to induce specific anti-toxin antibodies after injection. These diphtheria toxoids are used in diphtheria vaccines. Preferred diphtheria toxoids are those prepared by formaldehyde treatment. The diphtheria toxoid can be obtained by growing C. diphtheriae in growth medium, followed by formaldehyde treatment, ultrafiltration and precipitation. The toxoided material may then be treated by a process comprising sterile filtration and/or dialysis. The diphtheria toxoid is preferably adsorbed onto an aluminum hydroxide adjuvant.

Tetanus toxoid: Clostridium tetani causes tetanus. Tetanus toxin can be treated to give a protective toxoid. The toxoids are used in tetanus vaccines. Preferred tetanus toxoids are those prepared by formaldehyde treatment. The tetanus toxoid can be obtained by growing C. tetani in growth medium, followed by formaldehyde treatment, ultrafiltration and precipitation. The material may then be treated by a process comprising sterile filtration and/or dialysis.

Hepatitis A virus antigens: Hepatitis A virus (HAV) is one of the known agents which causes viral hepatitis. A preferred HAV component is based on inactivated virus, and inactivation can be achieved by formalin treatment.

Hepatitis B virus (HBV) is one of the known agents which causes viral hepatitis. The major component of the capsid is a protein known as HBV surface antigen or, more commonly, HBsAg, which is typically a 226-amino acid polypeptide with a molecular weight of ~24 kDa. All existing hepatitis B vaccines contain HBsAg, and when this antigen is administered to a normal vaccinee it stimulates the production of anti-HBsAg antibodies which protect against HBV infection.

For vaccine manufacture, HBsAg has been made in two ways: purification of the antigen in particulate form from the plasma of chronic hepatitis B carriers or expression of the protein by recombinant DNA methods (e.g., recombinant expression in yeast cells). Unlike native HBsAg (i.e., as in the plasma-purified product), yeast-expressed HBsAg is generally non-glycosylated, and this is the most preferred form of HBsAg for use with the invention.

Conjugated Haemophilus influenzae type b antigens: Haemophilus influenzae type b (Hib) causes bacterial meningitis. Hib vaccines are typically based on the capsular saccharide antigen, the preparation of which is well documented. The Hib saccharide can be conjugated to a carrier protein in order to enhance its immunogenicity, especially in children. Typical carrier proteins are tetanus toxoid, diphtheria toxoid, CRM_{197,} H. influenzae protein D, and an outer membrane protein complex from serogroup B meningococcus. The saccharide moiety of the conjugate may comprise full-length polyribosylribitol phosphate (PRP) as prepared from Hib bacteria, and/or fragments of full-length PRP. Hib conjugates may or may not be adsorbed to an aluminum salt adjuvant.

In an embodiment the vaccine formulations of the invention further include a conjugated N. meningitidis serogroup Y capsular saccharide (MenY), and/or a conjugated N. meningitidis serogroup C capsular saccharide (MenC).

In an embodiment the vaccine formulations of the invention further include a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the vaccine formulations of the invention further include a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

### Formulation

The formulations of the invention may be in liquid form (i.e., solutions or suspensions) or in a lyophilized form. Liquid formulations may advantageously be administered directly from their packaged form and are thus ideal for injection without the need for reconstitution in aqueous medium as otherwise required for lyophilized compositions of the invention.

Formulation of the composition of the present invention can be accomplished using art-recognized methods. For instance, the individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides formulations comprising any of combination of glycoconjugates disclosed herein and a pharmaceutically acceptable excipient, carrier, or diluent.

In an embodiment, the vaccine formulations of the invention are in liquid form, preferably in aqueous liquid form.

Vaccine formulations of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the vaccine formulations of the invention comprise a buffer. In an embodiment, said buffer has a pKa of about 3.5 to about 7.5. In some embodiments, the buffer is phosphate, succinate, histidine or citrate. In certain embodiments, the buffer is succinate at a final concentration of 1 mM to 10 mM. In one particular embodiment, the final concentration of the succinate buffer is about 5 mM

In an embodiment, the buffer is a succinate or histidine buffer. In an embodiment, the buffe is at a concentration of about 1 mM to 30 mM. In a preferred embodiment, the buffer is a succinate buffer having a final concentration of 1 mM to 10 mM. In a more preferred embodiment, the buffer is a succinate buffer having a final concentration of about 5 mM to 9 mM. In an embodiment, the buffer is a succinate buffer having a final concentration of about 1 mM. In an embodiment, the buffer is a succinate buffer having a final concentration of about 2 mM. In an embodiment, the buffer is a succinate buffer having a final concentration of about 3 mM. In an embodiment, the buffer is a succinate buffer having a final concentration of about 4 mM. In an embodiment, the buffer is a succinate buffer having a final concentration of about 5 mM. In an embodiment, the buffer is a succinate buffer having a final concentration of about 6 mM. In an embodiment, the buffer is a succinate buffer having a final concentration of about 7 mM. In an embodiment, the buffer is a succinate buffer having a final concentration of about 8 mM. In an embodiment, the buffer is a succinate buffer having a final concentration of about 9 mM. In an embodiment, the buffer is a succinate buffer having a final concentration of about 10 mM. In a preferred embodiment, the buffer is a succinate buffer having a final concentration of about 5 mM.

In an embodiment, the buffer is a histidine buffer. In an embodiment the histidine buffer is a histidine buffer having a final concentration of about 1 mM to 30 mM. In a preferred embodiment, the buffer is a histidine buffer having a final concentration of about 10 mM to 30 mM. In a more preferred embodiment, the buffer is a histidine buffer having a final concentration of about 20 mM to 30 mM. In an embodiment, the buffer is a histidine buffer having a final concentration of about 1 mM. In an embodiment, the buffer is a histidine buffer having a final concentration of about 2 mM. In an embodiment, the buffer is a histidine buffer having a final concentration of about, 3 mM. In an embodiment, the buffer is a histidine buffer having a final concentration of about 4 mM. In an embodiment, the buffer is a histidine buffer having a final concentration of about 5 mM. In an embodiment, the buffer is a histidine buffer having a final concentration of about 6 mM. In an embodiment, the buffer is a histidine buffer having a final concentration of about 7 mM. In an embodiment, the buffer is a histidine buffer having a final concentration of about 8 mM. In an embodiment, the buffer is a histidine buffer having a final concentration of about 9 mM. In an embodiment, the buffer is a histidine buffer having a final concentration of about 10 mM. In an embodiment, the buffer is a histidine buffer having a final concentration of about, 11 mM. In an embodiment, the buffer is a histidine buffer having a final concentration of about 12 mM. In an embodiment, the buffer is a histidine buffer having a final concentration of about 13 mM. In an embodiment, the buffer is a histidine buffer having a final concentration of about 14 mM. In an embodiment, the buffer is a histidine buffer having a final concentration of about 15 mM. In an embodiment, the buffer is a histidine buffer having a final concentration of about 16 mM. In an embodiment, the buffer is a histidine buffer having a final concentration of about 17 mM. In an embodiment, the buffer is a histidine buffer having a final concentration of about 18 mM. In an embodiment, the buffer is a histidine buffer having a final concentration of about 19 mM. In an embodiment, the buffer is a histidine buffer having a final concentration of about, 20 mM. In an embodiment, the buffer is a histidine buffer having a final concentration of about 21 mM. In an embodiment, the buffer is a histidine buffer having a final concentration of about 22 mM. In an embodiment, the buffer is a histidine buffer having a final concentration of about 23 mM. In an embodiment, the buffer is a histidine buffer having a final concentration of about 24 mM. In an embodiment, the buffer is a histidine buffer having a final concentration of about 25 mM. In an embodiment, the buffer is a histidine buffer having a final concentration of about 26 mM. In an embodiment, the buffer is a histidine buffer having a final concentration of about 27 mM. In an embodiment, the buffer is a histidine buffer having a final concentration of about 28, mM. In an embodiment, the buffer is a histidine buffer having a final concentration of about 29 mM. In an embodiment, the buffer is a histidine buffer having a final concentration of about 30 mM.

In a preferred embodiment, the buffer is a histidine buffer having a final concentration of 25 mM. In an embodiment, the buffer has a pH of about 5.0 to 7.5. In a preferred embodiment, the buffer has a pH of about 5.0 to 6.5. In a more preferred embodiment, the buffer has a pH of about 5.5 to 6.0. In an embodiment, the buffer has a pH of about 5.0. In an embodiment, the buffer has a pH of about 5.1. In an embodiment, the buffer has a pH of about 5.2. In an embodiment, the buffer has a pH of about 5.3. In an embodiment, the buffer has a pH of about 5.4. In an embodiment, the buffer has a pH of about 5.5. In an embodiment, the buffer has a pH of about 5.6. In an embodiment, the buffer has a pH of about 5.7. In an embodiment, the buffer has a pH of about 5.8. In an embodiment, the buffer has a pH of about 5.9. In an embodiment, the buffer has a pH of about 6.0. In an embodiment, the buffer has a pH of about 6.1. In an embodiment, the buffer has a pH of about 6.2. In an embodiment, the buffer has a pH of about 6.3. In an embodiment, the buffer has a pH of about 6.4. In an embodiment, the buffer has a pH of about 6.5. In an embodiment, the buffer has a pH of about 6.6. In an embodiment, the buffer has a pH of about 6.7. In an embodiment, the buffer has a pH of about 6.8. In an embodiment, the buffer has a pH of about 6.9. In an embodiment, the buffer has a pH of about 7.0. In an embodiment, the buffer has a pH of about 7.1. In an embodiment, the buffer has a pH of about 7.2. In an embodiment, the buffer has a pH of about 7.3. In an embodiment, the buffer has a pH of about 7.4. In an embodiment, the buffer has a pH of about 7.5. In a preferred embodiment, the buffer is a succinate or histidine buffer having a pH of 5.8.

In an embodiment, the formulations of the invention comprise a salt. In some embodiments, the salt is selected from the groups consisting of sodium phosphate, calcium chloride, magnesium chloride, potassium chloride, sodium chloride and a combination thereof. In one particular embodiment, the salt is sodium chloride. In one particular embodiment, the vaccine formulations of the invention comprise sodium chloride at 150 mM.

In an embodiment, the salt is sodium phosphate, calcium chloride, sodium chloride or combinations thereof. In an embodiment, the salt has a concentration of about 1 mM to 300 mM. In an embodiment, the salt is sodium chloride. In an embodiment, the salt is sodium chloride having a concentration of about 50 mM to 300 mM. In an embodiment, the salt is sodium chloride having a concentration of about 100 mM to 200 mM. In a preferred embodiment, the salt is sodium chloride having a concentration of about 200 mM to 300 mM. In a more preferred embodiment, the salt is sodium chloride having a concentration of about 150 mM to 250 mM. In an embodiment, the salt is sodium chloride having a concentration of about 50 mM. In an embodiment, the salt is sodium chloride having a concentration of about 75 mM. In an embodiment, the salt is sodium chloride having a concentration of about 100 mM. In an embodiment, the salt is sodium chloride having a concentration of about 125 mM. In an embodiment, the salt is sodium chloride having a concentration of about 150 mM. In an embodiment, the salt is sodium chloride having a concentration of about 175 mM. In an embodiment, the salt is sodium chloride having a concentration of about 200 mM. In an embodiment, the salt is sodium chloride having a concentration of about 225 mM. In an embodiment, the salt is sodium chloride having a concentration of about 250 mM. In an embodiment, the salt is sodium chloride having a concentration of about 275 mM. In an embodiment, the salt is sodium chloride having a concentration of about 300 mM. In an embodiment, the salt is sodium chloride having a concentration of about 125 mM. In an embodiment, the salt is sodium chloride having a concentration of about 130 mM. In an embodiment, the salt is sodium chloride having a concentration of about 135 mM. In an embodiment, the salt is sodium chloride having a concentration of about 140 mM, In an embodiment, the salt is sodium chloride having a concentration of about 145 mM. In an embodiment, the salt is sodium chloride having a concentration of about 150 mM. In an embodiment, the salt is sodium chloride having a concentration of about 155 mM. In an embodiment, the salt is sodium chloride having a concentration of about 160 mM. In an embodiment, the salt is sodium chloride having a concentration of about 165 mM. In an embodiment, the salt is sodium chloride having a concentration of about 170 mM. In an embodiment, the salt is sodium chloride having a concentration of about 175 mM. In an embodiment, the salt is sodium chloride having a concentration of about 225 mM. In an embodiment, the salt is sodium chloride having a concentration of about 230 mM. In an embodiment, the salt is sodium chloride having a concentration of about 235 mM. In an embodiment, the salt is sodium chloride having a concentration of about 240 mM. In an embodiment, the salt is sodium chloride having a concentration of about 245 mM. In an embodiment, the salt is sodium chloride having a concentration of about 250 mM. In an embodiment, the salt is sodium chloride having a concentration of about 255 mM. In an embodiment, the salt is sodium chloride having a concentration of about 260 mM. In an embodiment, the salt is sodium chloride having a concentration of about 265 mM. In an embodiment, the salt is sodium chloride having a concentration of about 270 mM. In an embodiment, the salt is sodium chloride having a concentration of about r 275 mM. In a particular embodiment, the salt is sodium chloride having a concentration of 150 mM. In a particular embodiment, the salt is sodium chloride having a concentration of 245 mM.

In an embodiment, the salt is magnesium chloride. In an embodiment, the salt is magnesium chloride having a concentration of about 10 mM to 50 mM. In a preferred embodiment, the salt is magnesium chloride having a concentration of about 20 mM to 50 mM. In a more preferred embodiment, the salt is magnesium chloride having a concentration of about 30 mM to 50 mM. In a particular embodiment, the salt is magnesium chloride having a concentration of about 35 mM to 45 mM. In an embodiment, the salt is magnesium chloride having a concentration of about 10 mM. In an embodiment, the salt is magnesium chloride having a concentration of about 15 mM. In an embodiment, the salt is magnesium chloride having a concentration of about 20 mM. In an embodiment, the salt is magnesium chloride having a concentration of about 25 mM. In an embodiment, the salt is magnesium chloride having a concentration of about 30 mM. In an embodiment, the salt is magnesium chloride having a concentration of about 35 mM. In an embodiment, the salt is magnesium chloride having a concentration of about 40 mM. In an embodiment, the salt is magnesium chloride having a concentration of about 45 mM. In an embodiment, the salt is magnesium chloride having a concentration of about 50 mM. In a particular embodiment, the salt is magnesium chloride having a concentration of about 40 mM.

In an embodiment the salt is calcium chloride. In an embodiment, the salt is calcium chloride having a concentration of about 1 mM to 50 mM. In a particular embodiment the salt is calcium chloride. In a preferred embodiment, the salt is calcium chloride having a concentration of about 10 mM to 30 mM. In a more preferred embodiment, the salt is calcium chloride having a concentration of about 15 mM to 25 mM. In an embodiment, the salt is calcium chloride having a concentration of about 5 mM. In an embodiment, the salt is calcium chloride having a concentration of about 10 mM. In an embodiment, the salt is calcium chloride having a concentration of about 15 mM. In an embodiment, the salt is calcium chloride having a concentration of about 20 mM. In an embodiment, the salt is calcium chloride having a concentration of about 25 mM. In an embodiment, the salt is calcium chloride having a concentration of about 30 mM. In an embodiment, the salt is calcium chloride having a concentration of about 35 mM. In an embodiment, the salt is calcium chloride having a concentration of about 40 mM. In a preferred embodiment, the salt is calcium chloride having a concentration of 20 mM.

In an embodiment the salt is sodium phosphate. In an embodiment, the salt is sodium phosphate having a concentration of about 1 mM to 50 mM. In a preferred embodiment, the salt is sodium phosphate having a concentration of about 20 mM to 50 mM. In a more preferred embodiment, the salt is sodium phosphate having a concentration of about 35 mM to 45 mM. In an embodiment, the salt is sodium phosphate having a concentration of about 5 mM. In an embodiment, the salt is sodium phosphate having a concentration of about 10 mM. In an embodiment, the salt is sodium phosphate having a concentration of about 15 mM. In an embodiment, the salt is sodium phosphate having a concentration of about 20 mM. In an embodiment, the salt is sodium phosphate having a concentration of about 25 mM. In an embodiment, the salt is sodium phosphate having a concentration of about 30 mM. In an embodiment, the salt is sodium phosphate having a concentration of about 35 mM. In an embodiment, the salt is sodium phosphate having a concentration of about 35 mM. In an embodiment, the salt is sodium phosphate having a concentration of about 40 mM. In an embodiment, the salt is sodium phosphate having a concentration of about 45 mM. In an embodiment, the salt is sodium phosphate having a concentration of about 50 mM. In a preferred embodiment, the salt is sodium phosphate having a concentration of 20 mM. In a preferred embodiment, the salt is sodium phosphate having a concentration of 40 mM.

In an embodiment, the salts are sodium phosphate and sodium chloride. In an embodiment, the sodium phosphate has a concentration of about 1 mM to 50 mM and the sodium chloride has a concentration of about 50 mM to 300 mM. In an embodiment, the sodium phosphate has a concentration of about 10 mM to 30 mM and the sodium chloride has a concentration of about 100 mM to −300 mM. In a preferred embodiment, the sodium phosphate has a concentration of about 15 mM to 25 mM and the sodium chloride has a concentration of about 200-300 mM. In a preferred embodiment, the sodium phosphate has a concentration of about 30 mM to 50 mM and the sodium chloride has a concentration of about 200-300 mM. In an embodiment, the sodium phosphate has a concentrate of about 5 mM. In an embodiment, the sodium phosphate has a concentrate of about 10 mM. In an embodiment, the sodium phosphate has a concentrate of about 15 mM. In an embodiment, the sodium phosphate has a concentrate of about 20 mM. In an embodiment, the sodium phosphate has a concentrate of about 25 mM. In an embodiment, the sodium phosphate has a concentrate of about In an embodiment, the sodium phosphate has a concentrate of about 35 mM. In an embodiment, the sodium phosphate has a concentrate of about 40 mM. In an embodiment, the sodium phosphate has a concentrate of about 45 mM. In an embodiment, the sodium phosphate has a concentrate of about 50 mM. In an embodiment, the sodium chloride has a concentration of about 125 mM. In an embodiment, the sodium chloride has a concentration of about 130 mM. In an embodiment, the sodium chloride has a concentration of about 135 mM. In an embodiment, the sodium chloride has a concentration of about 140 mM. In an embodiment, the sodium chloride has a concentration of about 145 mM. In an embodiment, the sodium chloride has a concentration of about 150 mM. In an embodiment, the sodium chloride has a concentration of about 155 mM. In an embodiment, the sodium chloride has a concentration of about 160 mM. In an embodiment, the sodium chloride has a concentration of about 165 mM. In an embodiment, the sodium chloride has a concentration of about 170 mM. In an embodiment, the sodium chloride has a concentration of about 175 mM. In an embodiment, the sodium chloride has a concentration of about 180 mM. In an embodiment, the sodium chloride has a concentration of about 185 mM. In an embodiment, the sodium chloride has a concentration of about 190 mM. In an embodiment, the sodium chloride has a concentration of about 200 mM. In an embodiment, the sodium chloride has a concentration of about 205 mM. In an embodiment, the sodium chloride has a concentration of about 210 mM. In an embodiment, the sodium chloride has a concentration of about 215 mM. In an embodiment, the sodium chloride has a concentration of about 220 mM. In an embodiment, the sodium chloride has a concentration of about 225 mM. In an embodiment, the sodium chloride has a concentration of about 230 mM. In an embodiment, the sodium chloride has a concentration of about 235 mM. In an embodiment, the sodium chloride has a concentration of about 240 mM. In an embodiment, the sodium chloride has a concentration of about 245 mM. In an embodiment, the sodium chloride has a concentration of about 250 mM. In an embodiment, the sodium chloride has a concentration of about 255 mM. In an embodiment, the sodium chloride has a concentration of about 260 mM. In an embodiment, the sodium chloride has a concentration of about 265 mM. In an embodiment, the sodium chloride has a concentration of about 270 mM. In an embodiment, the sodium chloride has a concentration of about 275 mM. In a particular embodiment, the sodium phosphate has a concentration of about 20 mM and the sodium chloride has a concentration of about 150 mM. In a particular embodiment, the salt is sodium phosphate having a concentration of 20 mM and sodium chloride having a concentration of 245 mM. In a particular embodiment, the salt is sodium phosphate having a concentration of 40 mM and sodium chloride having a concentration of 245 mM.

In an embodiment, the salts are sodium chloride and calcium chloride. In an embodiment, the sodium chloride has a concentration of about 50 to 300 mM and the calcium chloride has a concentration of about 1 mM to 50 mM. In a preferred embodiment, the sodium chloride has a concentration of about 100 to 250 mM and the calcium chloride has a concentration of about 20 mM to 30 mM. In a more preferred embodiment, the sodium chloride has a concentration of about 100 to 200 mM and the calcium chloride has a concentration of about 15 mM to 25 mM. In an embodiment, the sodium chloride has a concentration of about 125 mM. In an embodiment, the sodium chloride has a concentration of about 130 mM. In an embodiment, the sodium chloride has a concentration of about 135 mM. In an embodiment the sodium chloride has a concentration of about 140 mM. In an embodiment, the sodium chloride has a concentration of about 145 mM. In an embodiment, the sodium chloride has a concentration of about 150 mM. In an embodiment, the sodium chloride has a concentration of about 155 mM. In an embodiment, the sodium chloride has a concentration of about 160 mM. In an embodiment, the sodium chloride has a concentration of about 165 mM. In an embodiment, the sodium chloride has a concentration of about 170 mM. In an embodiment, the sodium chloride has a concentration of about 175 mM. In an embodiment, the calcium chloride has a concentration of about In an embodiment, the calcium chloride has a concentration of about 5 mM. In an embodiment, the calcium chloride has a concentration of about 10 mM. In an embodiment, the calcium chloride has a concentration of about 15 mM. In an embodiment, the calcium chloride has a concentration of about 20 mM. In an embodiment, the calcium chloride has a concentration of about 25 mM. In an embodiment, the calcium chloride has a concentration of about 30 mM. In an embodiment, the calcium chloride has a concentration of about 35 mM. In an embodiment, the calcium chloride has a concentration of about 40 mM. In a particular embodiment, the sodium chloride has a concentration of about 150 mM and the calcium chloride has a concentration of about 20 mM.

In an embodiment, the salts are sodium chloride and magnesium chloride. In an embodiment, the sodium chloride has a concentration of about 50 to 300 mM and the magnesium chloride has a concentration of 1 mM to 50 mM. In a preferred embodiment, the sodium chloride has a concentration of about 100 to 250 mM and the magnesium chloride has a concentration of 10 mM to 30 mM. In a more preferred embodiment, the sodium chloride has a concentration of about 100 to 200 mM and the magnesium chloride has a concentration of 15 mM to 25 mM. In an embodiment, the sodium chloride has a concentration of about 125 mM. In an embodiment, the sodium chloride has a concentration of about 130 mM. In an embodiment, the sodium chloride has a concentration of about 135 mM. In an embodiment, the sodium chloride has a concentration of about 140 mM. In an embodiment, the sodium chloride has a concentration of about 145 mM. In an embodiment, the sodium chloride has a concentration of about 150 mM. In an embodiment, the sodium chloride has a concentration of about 155 mM. In an embodiment, the sodium chloride has a concentration of about 160 mM. In an embodiment, the sodium chloride has a concentration of about 165 mM. In an embodiment, the sodium chloride has a concentration of about 170 mM. In an embodiment, the sodium chloride has a concentration of about 175 mM. In an embodiment, the magnesium chloride has a concentration of about 5 mM. In an embodiment, the magnesium chloride has a concentration of about 10 mM. In an embodiment, the magnesium chloride has a concentration of about 15 mM. In an embodiment, the magnesium chloride has a concentration of about 20 mM. In an embodiment, the magnesium chloride has a concentration of about 25 mM. In an embodiment, the magnesium chloride has a concentration of about 30 mM. In an embodiment, the magnesium chloride has a concentration of about 35 mM. In an embodiment, the magnesium chloride has a concentration of about 35 mM. In an embodiment, the magnesium chloride has a concentration of about 40 mM. In a particular embodiment, the sodium chloride has a concentration of about 150 mM and the magnesium chloride has a concentration of about 20 mM.

In an embodiment, the vaccine formulations of the invention comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer.

In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.001% to 1% polysorbate 80 weight to weight (w/w). In other embodiments, the final concentration of polysorbate 80 in the formulation is 0.01% polysorbate 80 (w/w). In other embodiments, the final concentration of polysorbate 80 in the formulation is 0.02% polysorbate 80 (w/w). In other embodiments, the final concentration of polysorbate 80 in the formulation is 0.03% polysorbate 80 (w/w). In other embodiments, the final concentration of polysorbate 80 in the formulation is 0.04% polysorbate 80 (w/w). In other embodiments, the final concentration of polysorbate 80 in the formulation is 0.05% polysorbate 80 (w/w). In other embodiments, the final concentration of polysorbate 80 in the formulation is 0.06% polysorbate 80 (w/w). In other embodiments, the final concentration of polysorbate 80 in the formulation is 0.07% polysorbate 80 (w/w). In other embodiments, the final concentration of polysorbate 80 in the formulation is 0.08%, In other embodiments, the final concentration of polysorbate 80 in the formulation is 0.09% polysorbate 80 (w/w). In other embodiments, the final concentration of polysorbate 80 in the formulation is 0.1% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.001% to 1% polysorbate 20 weight to weight (w/w). In other embodiments, the final concentration of polysorbate 20 in the formulation is 0.01% polysorbate 20 (w/w). In other embodiments, the final concentration of polysorbate 20 in the formulation is 0.02% polysorbate 20 (w/w). In other embodiments, the final concentration of polysorbate 20 in the formulation is 0.03% polysorbate 20 (w/w). In other embodiments, the final concentration of polysorbate 20 in the formulation is 0.04% polysorbate 20 (w/w). In other embodiments, the final concentration of polysorbate 20 in the formulation is 0.05% polysorbate 20 (w/w). In other embodiments, the final concentration of polysorbate 20 in the formulation is 0.06% polysorbate 20 (w/w). In other embodiments, the final concentration of polysorbate 20 in the formulation is 0.07% polysorbate 20 (w/w). In other embodiments, the final concentration of polysorbate 20 in the formulation is 0.08%, In other embodiments, the final concentration of polysorbate 20 in the formulation is 0.09% polysorbate 20 (w/w). In other embodiments, the final concentration of polysorbate 20 in the formulation is 0.1% polysorbate 20 (w/w). In another embodiment, the final concentration of the polysorbate 20 in the formulation is 1% polysorbate 20 (w/w).

In an embodiment, the formulations of the present invention include an adjuvant. Adjuvants of the formulations are described in detail below.

In an embodiment, the formulations of the present invention have a total glycoconjugate concentration of about 10 to 500 μg/ml. In an embodiment, the total glycoconjugate concentration is about 20 to 400 μg/ml. In an embodiment, the total glycoconjugate concentration is about 30 to 300 μg/ml. In a preferred embodiment, the total glycoconjugate concentration is about 50 to 200 μg/ml. In a more preferred embodiment, the total glycoconjugate concentration is about 100 to 150 μg/ml.

In an embodiment, the total glycoconjugate concentration is about 115 μg/ml. In an embodiment, the total glycoconjugate concentration is about 120 μg/ml. In an embodiment, the total glycoconjugate concentration is about 115 μg/ml. In an embodiment, the total glycoconjugate concentration is about 119 μg/ml.

In certain embodiments, the vaccine formulations of the invention have a pH of 5.5 to 7.5, more preferably a pH of 5.6 to 7.0, even more preferably a pH of 5.8 to 6.0.

In an embodiment, the present invention provides formulations including at least 21 different polysaccharide-protein conjugates; a succinic acid or a histidine buffer having a pH in the range of 5.0 to 7.5; calcium chloride, sodium chloride, calcium chloride and/or sodium phosphate; a surfactant; and an adjuvant. In an embodiment, the present invention provides formulations including at least 21 different polysaccharide-protein conjugates; a succinic acid buffer having a pH in the range of 5.0 to 7.5; calcium chloride; sodium chloride; a surfactant; and an adjuvant. In an embodiment, the present invention provides formulations including at least 21 different polysaccharide-protein conjugates; a succinic acid buffer having a pH in the range of 5.0 to 7.5; sodium chloride; sodium phosphate; a surfactant; and an adjuvant. In an embodiment, the present invention provides formulations including at least 21 different polysaccharide-protein conjugates; a histidine buffer having a pH in the range of 5.0 to 7.5; sodium chloride; a surfactant; and an adjuvant. In a preferred embodiment, the surfactant is polysorbate 80 or polysorbate 20. In a more preferred embodiment, the surfactant is polysorbate 80.

In an embodiment, the formulation includes 25 polysaccharide-protein conjugates, 5 mM succinate pH 5.8, 150 mM sodium chloride, 20 mM calcium chloride, 0.02% polysorbate 80 and 0.25 mg/ml aluminum phosphate. In an embodiment, the 25 polysaccharide-protein conjugates include one or more of S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.

In an embodiment, the formulation includes 25 polysaccharide-protein conjugates, 5 mM succinate pH 5.8, 40 mM sodium phosphate, 245 mM sodium chloride, 0.02% polysorbate 80 and 0.25 mg/ml aluminum phosphate. In an embodiment, the 25 polysaccharide-protein conjugates include one or more of S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.

In an embodiment, the formulation includes 25 polysaccharide-protein conjugates, 25 mM Histidine pH 5.8, 245 mM sodium chloride, 0.02% polysorbate 80 and 0.25 mg/ml aluminum phosphate. In an embodiment, the 25 polysaccharide-protein conjugates include one or more of S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.

In one embodiment, the present invention provides a container filled with any of the vaccine formulations disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present invention is made of glass, metals (e.g., steel, stainless steel, aluminum, etc.) and/or polymers (e.g., thermoplastics, elastomers, thermoplastic-elastomers).

In an embodiment, the container of the present invention is made of glass.

### Stability

In certain instances, it can be difficult to resuspend compositions or formulations that have been resting for a time (e.g., sitting on a shelf) that contain a significant number of glycoconjugates. Too much settlement or too dense of a settlement (e.g., too short of a “cake height,” as detailed below) can prevent resuspension of the glycoconjugates which can render a composition or formulation unusable or incapable of being injected. Further, if sedimentation occurs too quickly, such can interfere with manufacturing and the creation of a useful dosage form (e.g., the composition begins to settle before transference to a container). As detailed herein, in compositions or formulations containing a significant number of glycoconjugates, an embodiment of the present invention details a sedimentation rate that provides compositions that can more easily be manufactured for sale and/or resuspended for use.

Sedimentation velocity can be measured as described in the art. One method of measuring sedimentation rate is using a Turbiscan® TOWER. Turbiscan® TOWER uses static multiple light scattering to detect particle migration in liquid dispersions. A measurement head is outfitted with a pulsed near-infrared light source (2=880 nm), and synchronous transmission (180° from light source) and backscattering (45° from light source) detectors which move along the height of a flat-bottomed cylindrical glass sample cell, collecting data every 20 μm.

In certain embodiments, measurements were performed at room temperature using ~20 mL of sample. Samples were vortexed for resuspension immediately prior to the measurement. In certain embodiments, the measurement took place after the time after vortexing to positioning the sample in the scanner. The settling onset time is defined as the time where the sample reaches 45% clarification at the meniscus and was obtained from the transmission data. Sedimentation rate was reported as the slope of change in the sedimentation front position as a function of time.

In an embodiment, the present invention provides a composition including at least 25 different glycoconjugates and insoluble aluminum phosphate adjuvant, wherein at time T₀ substantially all of the at least 25 different glycoconjugates are dissolved in a liquid phase or adsorbed to the insoluble aluminum phosphate adjuvant as fully dispersed liquid suspension and the at least 25 different glycoconjugates are at a concentration C₀ in the liquid phase; at time T₁ a portion of the at least 25 different glycoconjugates adsorbed to the insoluble adjuvant sediment from the liquid phase to form a sediment and the at least 25 different glycoconjugates are at a concentration C₁ in the liquid phase; at time T₂ additional portion of the at least 25 different glycoconjugates adsorbed to the insoluble adjuvant sediment from the liquid phase to form a sediment and the at least 25 different glycoconjugates are at a concentration C₂ in the liquid phase; and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the height of a flat-bottomed cylindrical glass sample cell, collecting precipitate data every 20 μm.

In one embodiment, the composition includes at least 21 different glycoconjugates. In one embodiment, the composition includes at least 22 different glycoconjugates. In one embodiment, the composition includes at least 23 different glycoconjugates. In one embodiment, the composition includes at least 24 different glycoconjugates. In one embodiment, the composition includes at least 25 different glycoconjugates. In one embodiment, the composition includes at least 26 different glycoconjugates. In one embodiment, the composition includes at least 27 different glycoconjugates. In one embodiment, the composition includes at least 28 different glycoconjugates. In one embodiment, the composition includes at least 29 different glycoconjugates. In one embodiment, the composition includes at least 30 different glycoconjugates. In one embodiment, the composition includes at least 31 different glycoconjugates. In one embodiment, the composition includes at least 32 different glycoconjugates. In one embodiment, the composition includes at least 33 different glycoconjugates. In one embodiment, the composition includes at least 34 different glycoconjugates. In one embodiment, the composition includes at least 35 different glycoconjugates.

In an embodiment, T₀ is 0 hour. In an embodiment, T₁ is about 0.01 hours to 4 hours. In an embodiment, T₁ is about 1 hour to 2 hours. In a preferred embodiment, T₁ is about 0.01 hours to 4 hours. In an embodiment, T₁ is about 0.1 hour. In an embodiment, T₁ is about 0.2 hour. In an embodiment, T₁ is about 0.3 hour. In an embodiment, T₁ is about 0.4 hour. In an embodiment, T₁ is about 0.5 hour. In an embodiment, T₁ is about 0.6 hour. In an embodiment, T₁ is about 0.7 hour. In an embodiment, T₁ is about 0.8 hour. In an embodiment, T₁ is about 0.9 hour. In an embodiment, T₁ is about 1.0 hour. In an embodiment, T₁ is about 1.1 hours. In an embodiment, T₁ is about 1.2 hours. In an embodiment, T₁ is about 1.3 hours. In an embodiment, T₁ is about 1.4 hours. In an embodiment, T₁ is about 1.5 hours. In an embodiment, T₁ is about 1.6 hours. In an embodiment, T₁ is about 1.7 hours. In an embodiment, T₁ is about 1.8 hours. In an embodiment, T₁ is about 1.9 hours. In an embodiment, T₁ is about 2.0 hours. In an embodiment, T₁ is about 2.1 hours. In an embodiment, T₁ is about 2.2 hours. In an embodiment, T₁ is about 2.3 hours. In an embodiment, T₁ is about 2.4 hours. In an embodiment, T₁ is about 2.5 hours. In an embodiment, T₁ is about 2.6 hours. In an embodiment, T₁ is about 2.7 hours. In an embodiment, T₁ is about 2.8 hours. In an embodiment, T₁ is about 2.9 hours. In an embodiment, T₁ is about 3.0 hours. In an embodiment, T₁ is about 3.1 hours. In an embodiment, T₁ is about 3.2 hours. In an embodiment, T₁ is about 3.3 hours. In an embodiment, T₁ is about 3.4 hours. In an embodiment, T₁ is about 3.5 hours. In an embodiment, T₁ is about 3.6 hours. In an embodiment, T₁ is about 3.7 hours. In an embodiment, T₁ is about 3.8 hours. In an embodiment, T₁ is about 3.9 hours. In an embodiment, T₁ is about 4.0 hours.

In an embodiment, T₂ is about 1 hour to 5 hours. In a preferred embodiment, T₂ is about 1 to 3 hours. In a more preferred embodiment, T₂ is about 1 to 2 hours. In a particular embodiment, T₂ is about 4 hours. In an embodiment, T₂ is about 1.0 hour. In an embodiment, T₂ is about 1.1 hours. In an embodiment, T₂ is about 1.2 hours. In an embodiment, T₂ is about 1.3 hours. In an embodiment, T₂ is about 1.4 hours. In an embodiment, T₂ is about 1.5 hours. In an embodiment, T₂ is about 1.6 hours. In an embodiment, T₂ is about 1.7 hours. In an embodiment, T₂ is about 1.8 hours. In an embodiment, T₂ is about 1.9 hours. In an embodiment, T₂ is about 2.0 hours. In an embodiment, T₂ is about 2.1 hours. In an embodiment, T₂ is about 2.2 hours. In an embodiment, T₂ is about 2.3 hours. In an embodiment, T₂ is about 2.4 hours. In an embodiment, T₂ is about 2.5 hours. In an embodiment, T₂ is about 2.6 hours. In an embodiment, T₂ is about 2.7 hours. In an embodiment, T₂ is about 2.8 hours. In an embodiment, T₂ is about 2.9 hours. In an embodiment, T₂ is about 3.0 hours. In an embodiment, T₂ is about 3.1 hours. In an embodiment, T₂ is about 3.2 hours. In an embodiment, T₂ is about 3.3 hours. In an embodiment, T₂ is about 3.4 hours. In an embodiment, T₂ is about 3.6 hours. In an embodiment, T₂ is about 3.7 hours. In an embodiment, T₂ is about 3.8 hours. In an embodiment, T₂ is about 3.9 hours. In an embodiment, T₂ is about 4.0 hours. In an embodiment, T₂ is about 4.1 hours. In an embodiment, T₂ is about 4.2 hours. In an embodiment, T₂ is about 4.3 hours. In an embodiment, T₂ is about 4.4 hours. In an embodiment, T₂ is about 4.5 hours. In an embodiment, T₂ is about 4.6 hours. In an embodiment, T₂ is about 4.7 hours. In an embodiment, T₂ is about 4.8 hours. In an embodiment, T₂ is about 4.9 hours. In an embodiment, T₂ is about 5 hours.

In an embodiment, C₀ is greater than C₁ and C₂. In an embodiment, C₁ is greater than C₂. In one embodiment, C₀ is greater than C₁ and C₂. In one embodiment, C₁ is greater than C₂.

In an embodiment, C₀ is greater than C₁ and C₂. In an embodiment, C₁ is greater than C₂. In one embodiment, C₀ is greater than C₁ and C₂. In one embodiment, C₁ is greater than C₂. In one embodiment, C₀ is greater than C₁ and C₂. In one embodiment, C₁ is greater than C₂.

In an embodiment, at T₁ peak thickness of the sedimentation front is about 0 mm to 20.0 mm. In a preferred embodiment, at T₁ peak thickness of the sedimentation front is about 1 mm to 10.0 mm. In a preferred embodiment, at T₁ peak thickness of the sedimentation front is about 1 mm to 5.0 mm. In a more preferred embodiment, at T₁ peak thickness of the sedimentation front is at least 2 mm. In an embodiment, at T₁ peak thickness of the sedimentation front is about 0 mm. In an embodiment, at T₁ peak thickness of the sedimentation front is about 0.1 mm. In an embodiment, at T₁ peak thickness of the sedimentation front is about 0.2 mm. In an embodiment, at T₁ peak thickness of the sedimentation front is about 0.3 mm. In an embodiment, at T₁ peak thickness of the sedimentation front is about 0.4 mm. In an embodiment, at T₁ peak thickness of the sedimentation front is about 0.5 mm. In an embodiment, at T₁ peak thickness of the sedimentation front is about 0.6 mm. In an embodiment, at T₁ peak thickness of the sedimentation front is about 0.7 mm. In an embodiment, at T₁ peak thickness of the sedimentation front is about 0.8 mm. In an embodiment, at T₁ peak thickness of the sedimentation front is about 0.9 mm. In an embodiment, at T₁ peak thickness of the sedimentation front is about 1.0 mm. In an embodiment, at T₁ peak thickness of the sedimentation front is about 2.0 mm. In an embodiment, at T₁ peak thickness of the sedimentation front is about 3.0 mm. In an embodiment, at T₁ peak thickness of the sedimentation front is about 4.0 mm. In an embodiment, at T₁ peak thickness of the sedimentation front is about 5.0 mm. In an embodiment, at T₁ peak thickness of the sedimentation front is about 6.0 mm. In an embodiment, at T₁ peak thickness of the sedimentation front is about 7.0 mm. In an embodiment, at T₁ peak thickness of the sedimentation front is about 8.0 mm. In an embodiment, at T₁ peak thickness of the sedimentation front is about 9.0 mm. In an embodiment, at T₁ peak thickness of the sedimentation front is about 10.0 mm. In an embodiment, at T₁ peak thickness of the sedimentation front is about 11.0 mm. In an embodiment, at T₁ peak thickness of the sedimentation front is about 12.0 mm. In an embodiment, at T₁ peak thickness of the sedimentation front is about 13.0 mm. In an embodiment, at T₁ peak thickness of the sedimentation front is about 14.0 mm. In an embodiment, at T₁ peak thickness of the sedimentation front is about 15.0 mm. In an embodiment, at T₁ peak thickness of the sedimentation front is about 16.0 mm. In an embodiment, at T₁ peak thickness of the sedimentation front is about 17.0 mm. In an embodiment, at T₁ peak thickness of the sedimentation front is about 18.0 mm. In an embodiment, at T₁ peak thickness of the sedimentation front is about 19.0 mm. In an embodiment, at T₁ peak thickness of the first solid phase sediment is about 20.0 mm. In an embodiment, at T₁ peak thickness of the sedimentation front is about 20.0 mm.

In an embodiment, at T₂ peak thickness of the sedimentation front is about 2 mm to 25.0 mm. In a preferred embodiment, at T₂ peak thickness of the sedimentation front is about 5 mm to 20.0 mm. In a more preferred embodiment, at T₂ peak thickness of the sedimentation front is about 5 mm to 15.0 mm. In an embodiment, at T₂ peak thickness of the sedimentation front is at least 10 mm. In an embodiment, at T₁ peak thickness of the sedimentation front is about 0 mm. In an embodiment, at T₂ peak thickness of the sedimentation front is at least 1.0 mm. In an embodiment, at T₂ peak thickness of the sedimentation front is at least 2.0 mm. In an embodiment, at T₂ peak thickness of the sedimentation front is at least 3.0 mm. In an embodiment, at T₂ peak thickness of the sedimentation front is at least 4.0 mm. In an embodiment, at T₂ peak thickness of the sedimentation front is at least 5.0 mm. In an embodiment, at T₂ peak thickness of the sedimentation front is at least 6.0 mm. In an embodiment, at T₂ peak thickness of the sedimentation front is at least 7.0 mm. In an embodiment, at T₂ peak thickness of the sedimentation front is at least 8.0 mm. In an embodiment, at T₂ peak thickness of the sedimentation front is at least 9.0 mm. In an embodiment, at T₂ peak thickness of the sedimentation front is at least 10.0 mm. In an embodiment, at T₂ peak thickness of the sedimentation front is at least 11.0 mm. In an embodiment, at T₂ peak thickness of the sedimentation front is at least 12.0 mm. In an embodiment, at T₂ peak thickness of the sedimentation front is at least 13.0 mm. In an embodiment, at T₂ peak thickness of the sedimentation front is at least 14.0 mm. In an embodiment, at T₂ peak thickness of the sedimentation front is at least 15.0 mm. In an embodiment, at T₂ peak thickness of the sedimentation front is at least 16.0 mm. In an embodiment, at T₂ peak thickness of the sedimentation front is at least 17.0 mm. In an embodiment, at T₂ peak thickness of the sedimentation front is at least 18.0 mm. In an embodiment, at T₂ peak thickness of the sedimentation front is at least 19.0 mm. In an embodiment, at T₂ peak thickness of the sedimentation front is at least 20.0 mm. In an embodiment, at T₂ peak thickness of the sedimentation front is at least 21.0 mm. In an embodiment, at T₂ peak thickness of the sedimentation front is at least 22.0 mm. In an embodiment, at T₂ peak thickness of the sedimentation front is at least 23.0 mm. In an embodiment, at T₂ peak thickness of the sedimentation front is at least 24.0 mm. In an embodiment, at T₂ peak thickness of the sedimentation front is at least 25.0 mm.

In an embodiment, the sedimentary velocity of the sedimentation front is less than a peak thickness of 10 mm at about 1 hour and is greater than a peak thickness of 18 mm at about 4 hours.

In an embodiment, the invention further includes a time T₃ wherein the insoluble aluminum phosphate adsorbed glycoconjugate sedimentation is at equilibrium with the liquid phase at T₃. In an embodiment, T₃ is about 2 hours to 5 hours. In a preferred embodiment, T₃ is about 3 hours to 5 hours. In a more preferred embodiment, T₃ is about 4 hours to 5 hours. In an embodiment, T₃ is about 2.0 hours. In an embodiment, T₃ is about 2.1 hours. In an embodiment, T₃ is about 2.2 hours. In an embodiment, T₃ is about 2.3 hours. In an embodiment, T₃ is about 2.4 hours. In an embodiment, T₃ is about 2.5 hours. In an embodiment, T₃ is about 2.6 hours. In an embodiment, T₃ is about 2.7 hours. In an embodiment, T₃ is about 2.8 hours. In an embodiment, T₃ is about 2.9 hours. In an embodiment, T₃ is about 3.0 hours. In an embodiment, T₃ is about 3.1 hours. In an embodiment, T₃ is about 3.2 hours. In an embodiment, T₃ is about 3.3 hours. In an embodiment, T₃ is about 3.4 hours. In an embodiment, T₃ is about 3.5 hours. In an embodiment, T₃ is about 3.6 hours. In an embodiment, T₃ is about 3.7 hours. In an embodiment, T₃ is about 3.8 hours. In an embodiment, T₃ is about 3.9 hours. In an embodiment, T₃ is about 4.0 hours. In an embodiment, T₃ is about 4.1 hours. In an embodiment, T₃ is about 4.2 hours. In an embodiment, T₃ is about In an embodiment, T₃ is about 4.4 hours. In an embodiment, T₃ is about 4.5 hours. In an embodiment, T₃ is about 4.6 hours. In an embodiment, T₃ is about 4.7 hours. In an embodiment, T₃ is about 4.8 hours. In an embodiment, T₃ is about 4.9 hours. In an embodiment, T₃ is about 5 hours.

In an embodiment, at T₃ the sedimentation front is about 25 mm to 40 mm. In a preferred embodiment, at T₃ the sedimentation front is about 30 mm to 40 mm. In a more preferred embodiment, at T₃ the sedimentation front is about 35 mm to 40 mm. In an embodiment, T₃ is about 25.0 mm. In an embodiment, T₃ is about 26 mm. In an embodiment, T₃ is about 27 mm. In an embodiment, T₃ is about 28 mm. In an embodiment, T₃ is about 29 mm. In an embodiment, T₃ is about 30 mm. In an embodiment, T₃ is about 31 mm. 3 In an embodiment, T₃ is about 2 mm. In an embodiment, T₃ is about 33 mm. In an embodiment, T₃ is about 34 mm. In an embodiment, T₃ is about 35 mm. In an embodiment, T₃ is about 36 mm. In an embodiment, T₃ is about 37 mm. In an embodiment, T₃ is about 38 mm. In an embodiment, T₃ is about 39 mm. In an embodiment, T₃ is about 40 mm.

In an embodiment, the composition has been at rest for about 1 month. In an embodiment, the composition has been at rest for at least 2 weeks. In an embodiment, the composition is stored in a container. In an embodiment, the container is a syringe.

In an embodiment, where after T₃ the composition is resuspended with about 1-10 handshakes. In a preferred embodiment, where after T₃ the composition is resuspended with about 1-5 handshakes. In a more preferred embodiment, after T₃ the composition is resuspended with about 1-3 handshakes. In an embodiment, after T₃ the composition is resuspended with about 1 handshake. In an embodiment, after T₃ the composition is resuspended with about 2 handshakes. In an embodiment, after T₃ the composition is resuspended with about 3 handshakes. In an embodiment, after T₃ the composition is resuspended with about 4 handshakes. 5 handshakes. In an embodiment, after T₃ the composition is resuspended with about 6 handshakes. In an embodiment, after T₃ the composition is resuspended with about 7 handshakes. In an embodiment, after T₃ the composition is resuspended with about 8 handshakes. In an embodiment, after T₃ the composition is resuspended with about 9 handshakes. In an embodiment, after T₃ the composition is resuspended with about 10 handshakes. In an embodiment, the composition comprises the formulation previously described.

In an embodiment, the present invention provides a liquid filled container including at least 25 different glycoconjugates and insoluble aluminum phosphate adjuvant, wherein: at time To, substantially all of the at least 25 different glycoconjugates are dissolved in a liquid phase or adsorbed to the insoluble aluminum phosphate adjuvant as fully dispersed liquid suspension and the at least 25 different glycoconjugates are at a concentration C₀ in the liquid phase; at time T₁, a portion of the at least 25 different glycoconjugates adsorbed to the insoluble adjuvant sediment from the liquid phase to form a sediment and the at least 25 different glycoconjugates has a concentration C₁ in the liquid phase; at time T₂ additional portion of the at least 25 different glycoconjugates adsorbed to the insoluble adjuvant sediment from the liquid phase to form a sediment and the at least 25 different glycoconjugates are at a concentration C₂ in the liquid phase; and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the thickness of a flat-bottomed cylindrical glass sample cell, collecting precipitate data every 20 μm.

In one embodiment, the container includes at least 21 different glycoconjugates. In one embodiment, the container includes at least 22 different glycoconjugates. In one embodiment, the container includes at least 23 different glycoconjugates. In one embodiment, the container includes at least 24 different glycoconjugates. In one embodiment, the container includes at least 25 different glycoconjugates. In one embodiment, the container includes at least 26 different glycoconjugates. In one embodiment, the container includes at least 27 different glycoconjugates. In one embodiment, the container includes at least 28 different glycoconjugates. In one embodiment, the container includes at least 29 different glycoconjugates. In one embodiment, the container includes at least 30 different glycoconjugates. In one embodiment, the container includes at least 31 different glycoconjugates. In one embodiment, the container includes at least 32 different glycoconjugates. In one embodiment, the container includes at least 33 different glycoconjugates. In one embodiment, the container includes at least 34 different glycoconjugates. In one embodiment, the container includes at least 35 different glycoconjugates.

In an embodiment, T₀ is 0 hour. In an embodiment, T₁ is about 0.01 hours to 4 hours. In a preferred embodiment, T₁ is about 1 hours to 3 hours. In a more preferred embodiment, T₁ is about 1 hour to 2 hours. In an embodiment, T₁ is about 0.1 hour. In an embodiment, T₁ is about 0.2 hour. In an embodiment, T₁ is about 0.3 hour. In an embodiment, T₁ is about 0.4 hour. In an embodiment, T₁ is about 0.5 hour. In an embodiment, T₁ is about 0.6 hour. In an embodiment, T₁ is about 0.7 hour. In an embodiment, T₁ is about 0.8 hour. In an embodiment, T₁ is about 0.9 hour. In an embodiment, T₁ is about 1.0 hour. In an embodiment, T₁ is about 1.1 hours. In an embodiment, T₁ is about 1.2 hours. In an embodiment, T₁ is about 1.3 hours. In an embodiment, T₁ is about 1.4 hours. In an embodiment, T₁ is about 1.5 hours. In an embodiment, T₁ is about 1.6 hours. In an embodiment, T₁ is about 1.7 hours. In an embodiment, T₁ is about 1.8 hours. In an embodiment, T₁ is about 1.9 hours. In an embodiment, T₁ is about 2.0 hours. In an embodiment, T₁ is about 2.1 hours. In an embodiment, T₁ is about 2.2 hours. In an embodiment, T₁ is about 2.3 hours. In an embodiment, T₁ is about 2.4 hours. In an embodiment, T₁ is about 2.5 hours. In an embodiment, T₁ is about 2.6 hours. In an embodiment, T₁ is about 2.7 hours. In an embodiment, T₁ is about 2.8 hours. In an embodiment, T₁ is about 2.9 hours. In an embodiment, T₁ is about 3.0 hours. In an embodiment, T₁ is about 3.1 hours. In an embodiment, T₁ is about 3.2 hours. In an embodiment, T₁ is about 3.3 hours. In an embodiment, T₁ is about 3.4 hours. In an embodiment, T₁ is about 3.5 hours. In an embodiment, T₁ is about 3.6 hours. In an embodiment, T₁ is about 3.7 hours. In an embodiment, T₁ is about 3.8 hours. In an embodiment, T₁ is about 3.9 hours. In an embodiment, T₁ is about 4.0 hours.

In an embodiment, T₂ is about 1 hour to 5 hours. In a preferred embodiment, T₂ is about 1 hour to 3 hours. In a more preferred embodiment, T₂ is about 1 hour to 2 hours. In a particular embodiment, T₂ is about 4 hours. In an embodiment, T₂ is about 1.0 hour. In an embodiment, T₂ is about 1.1 hours. In an embodiment, T₂ is about 1.2 hours. In an embodiment, T₂ is about 1.3 hours. In an embodiment, T₂ is about 1.4 hours. In an embodiment, T₂ is about 1.5 hours. In an embodiment, T₂ is about 1.6 hours. In an embodiment, T₂ is about 1.7 hours. In an embodiment, T₂ is about 1.8 hours. In an embodiment, T₂ is about 1.9 hours. In an embodiment, T₂ is about 2.0 hours. In an embodiment, T₂ is about 2.1 hours. In an embodiment, T₂ is about 2.2 hours. In an embodiment, T₂ is about 2.3 hours. In an embodiment, T₂ is about 2.4 hours. In an embodiment, T₂ is about 2.5 hours. In an embodiment, T₂ is about 2.6 hours. In an embodiment, T₂ is about 2.7 hours. In an embodiment, T₂ is about 2.8 hours. In an embodiment, T₂ is about 2.9 hours. In an embodiment, T₂ is about 3.0 hours. In an embodiment, T₂ is about 3.1 hours. In an embodiment, T₂ is about 3.2 hours. In an embodiment, T₂ is about 3.3 hours. In an embodiment, T₂ is about 3.4 hours. In an embodiment, T₂ is about 3.6 hours. In an embodiment, T₂ is about 3.7 hours. In an embodiment, T₂ is about 3.8 hours. In an embodiment, T₂ is about 3.9 hours. In an embodiment, T₂ is about 4.0 hours. In an embodiment, T₂ is about 4.1 hours. In an embodiment, T₂ is about 4.2 hours. In an embodiment, T₂ is about 4.3 hours. In an embodiment, T₂ is about 4.4 hours. In an embodiment, T₂ is about 4.5 hours. In an embodiment, T₂ is about 4.6 hours. In an embodiment, T₂ is about 4.7 hours. In an embodiment, T₂ is about 4.8 hours. In an embodiment, T₂ is about 4.9 hours. In an embodiment, T₂ is about 5 hours.

In an embodiment, C₀ is greater than C₁ and C₂. In an embodiment, C₁ is greater than C₂. In one embodiment, C₀ is greater than C₁ and C₂. In one embodiment, C₁ is greater than C₂.

In an embodiment, C₀ is greater than C₁ and C₂. In an embodiment, C₁ is greater than C₂. In one embodiment, C₀ is greater than C₁ and C₂. In one embodiment, C₁ is greater than C₂. In one embodiment, C₀ is greater than C₁ and C₂. In one embodiment, C₁ is greater than C₂.

In an embodiment, at T₁ peak thickness of the sedimentation front is about 0 mm to 20.0 mm. In a preferred embodiment, at T₁ peak thickness of the sedimentation front is about 1 mm to 10.0 mm. In a more preferred embodiment, at T₁ peak thickness of the sedimentation front is about 1 mm to 5.0 mm. In a particular embodiment, at T₁ peak thickness of the sedimentation front is at least 2 mm. In an embodiment, at T₁ peak thickness of the sedimentation front is about 0 mm. In an embodiment, at T₁ peak thickness of the sedimentation front is about 0.1 mm. In an embodiment, at T₁ peak thickness of the sedimentation front is about 0.2 mm. In an embodiment, at T₁ peak thickness of the sedimentation front is about 0.3 mm. In an embodiment, at T₁ peak thickness of the sedimentation front is about 0.4 mm. In an embodiment, at T₁ peak thickness of the sedimentation front is about 0.5 mm. In an embodiment, at T₁ peak thickness of the sedimentation front is about 0.6 mm. In an embodiment, at T₁ peak thickness of the sedimentation front is about 0.7 mm. In an embodiment, at T₁ peak thickness of the sedimentation front is about 0.8 mm. In an embodiment, at T₁ peak thickness of the sedimentation front is about 0.9 mm. In an embodiment, at T₁ peak thickness of the sedimentation front is about 1.0 mm. In an embodiment, at T₁ peak thickness of the sedimentation front is about 2.0 mm. In an embodiment, at T₁ peak thickness of the sedimentation front is about 3.0 mm. In an embodiment, at T₁ peak thickness of the sedimentation front is about 4.0 mm. In an embodiment, at T₁ peak thickness of the sedimentation front is about 5.0 mm. In an embodiment, at T₁ peak thickness of the sedimentation front is about 6.0 mm. In an embodiment, at T₁ peak thickness of the sedimentation front is about 7.0 mm. In an embodiment, at T₁ peak thickness of the sedimentation front is about 8.0 mm. In an embodiment, at T₁ peak thickness of the sedimentation front is about 9.0 mm. In an embodiment, at T₁ peak thickness of the sedimentation front is about 10.0 mm. In an embodiment, at T₁ peak thickness of the sedimentation front is about 11.0 mm. In an embodiment, at T₁ peak thickness of the sedimentation front is about 12.0 mm. In an embodiment, at T₁ peak thickness of the sedimentation front is about 13.0 mm. In an embodiment, at T₁ peak thickness of the sedimentation front is about 14.0 mm. In an embodiment, at T₁ peak thickness of the sedimentation front is about 15.0 mm. In an embodiment, at T₁ peak thickness of the sedimentation front is about 16.0 mm. In an embodiment, at T₁ peak thickness of the sedimentation front is about 17.0 mm. In an embodiment, at T₁ peak thickness of the sedimentation front is about 18.0 mm. In an embodiment, at T₁ peak thickness of the sedimentation front is about 19.0 mm. In an embodiment, at T₁ peak thickness of the sedimentation front is about 20.0 mm.

In an embodiment, at T₂ peak thickness of the sedimentation front is about 2 mm to 25.0 mm. In a preferred embodiment, at T₂ peak thickness of the sedimentation front is about 5 mm to 20.0 mm. In a more preferred embodiment, at T₂ peak thickness of the sedimentation front is about 5 mm to 15.0 mm. In a particular embodiment, at T₂ peak thickness of the sedimentation front is at least 10 mm. In an embodiment, at T₁ peak thickness of the sedimentation front is about 0 mm. In an embodiment, at T₂ peak thickness of the sedimentation front is at least 1.0 mm. In an embodiment, at T₂ peak thickness of the sedimentation front is at least 2.0 mm. In an embodiment, at T₂ peak thickness of the sedimentation front is at least 3.0 mm. In an embodiment, at T₂ peak thickness of the sedimentation front is at least 4.0 mm. In an embodiment, at T₂ peak thickness of the sedimentation front is at least 5.0 mm. In an embodiment, at T₂ peak thickness of the sedimentation front is at least 6.0 mm. In an embodiment, at T₂ peak thickness of the sedimentation front is at least 7.0 mm. In an embodiment, at T₂ peak thickness of the sedimentation front is at least 8.0 mm. In an embodiment, at T₂ peak thickness of the sedimentation front is at least 9.0 mm. In an embodiment, at T₂ peak thickness of the sedimentation front is at least 10.0 mm. In an embodiment, at T₂ peak thickness of the sedimentation front is at least 11.0 mm. In an embodiment, at T₂ peak thickness of the sedimentation front is at least 12.0 mm. In an embodiment, at T₂ peak thickness of the sedimentation front is at least 13.0 mm. In an embodiment, at T₂ peak thickness of the sedimentation front is at least 14.0 mm. In an embodiment, at T₂ peak thickness of the sedimentation front is at least 15.0 mm. In an embodiment, at T₂ peak thickness of the sedimentation front is at least 16.0 mm. In an embodiment, at T₂ peak thickness of the sedimentation front is at least 17.0 mm. In an embodiment, at T₂ peak thickness of the sedimentation front is at least 18.0 mm. In an embodiment, at T₂ peak thickness of the sedimentation front is at least 19.0 mm. In an embodiment, at T₂ peak thickness of the sedimentation front is at least 20.0 mm. In an embodiment, at T₂ peak thickness of the sedimentation front is at least 21.0 mm. In an embodiment, at T₂ peak thickness of the sedimentation front is at least 22.0 mm. In an embodiment, at T₂ peak thickness of the sedimentation front is at least 23.0 mm. In an embodiment, at T₂ peak thickness of the sedimentation front is at least 24.0 mm. In an embodiment, at T₂ peak thickness of the sedimentation front is at least 25.0 mm.

In an embodiment, the sedimentary velocity of the sedimentation front is less than a peak thickness of 10 mm at about 1 hour and is greater than a peak thickness of 18 mm at about 4 hours.

In an embodiment, the invention further includes a time T₃ wherein the insoluble aluminum phosphate adsorbed glycoconjugate sedimentation is at equilibrium with the liquid phase at T₃. In an embodiment, T₃ is about 2 hours to 5 hours. In a preferred embodiment, T₃ is about 3 hours to 5 hours. In a more preferred embodiment, T₃ is about 4 hours to 5 hours. In an embodiment, T₃ is about 2.0 hours. In an embodiment, T₃ is about 2.1 hours. In an embodiment, T₃ is about 2.2 hours. In an embodiment, T₃ is about 2.3 hours. In an embodiment, T₃ is about 2.4 hours. In an embodiment, T₃ is about 2.5 hours. In an embodiment, T₃ is about 2.6 hours. In an embodiment, T₃ is about 2.7 hours. In an embodiment, T₃ is about 2.8 hours. In an embodiment, T₃ is about 2.9 hours. In an embodiment, T₃ is about 3.0 hours. In an embodiment, T₃ is about 3.1 hours. In an embodiment, T₃ is about 3.2 hours. In an embodiment, T₃ is about 3.3 hours. In an embodiment, T₃ is about 3.4 hours. In an embodiment, T₃ is about 3.5 hours. In an embodiment, T₃ is about 3.6 hours. In an embodiment, T₃ is about 3.7 hours. In an embodiment, T₃ is about 3.8 hours. In an embodiment, T₃ is about 3.9 hours. In an embodiment, T₃ is about 4.0 hours. In an embodiment, T₃ is about 4.1 hours. In an embodiment, T₃ is about 4.2 hours. In an embodiment, T₃ is about In an embodiment, T₃ is about 4.4 hours. In an embodiment, T₃ is about 4.5 hours. In an embodiment, T₃ is about 4.6 hours. In an embodiment, T₃ is about 4.7 hours. In an embodiment, T₃ is about 4.8 hours. In an embodiment, T₃ is about 4.9 hours. In an embodiment, T₃ is about 5 hours.

In an embodiment, at T₃ the sedimentation front is about 25 mm to 40 mm. In a preferred embodiment, at T₃ the sedimentation front is about 30 mm to 40 mm. In a more preferred embodiment, at T₃ the sedimentation front is about 35 mm to 40 mm. In an embodiment, T₃ is about 25.0 mm. In an embodiment, T₃ is about 26 mm. In an embodiment, T₃ is about 27 mm. In an embodiment, T₃ is about 28 mm. In an embodiment, T₃ is about 29 mm. In an embodiment, T₃ is about 30 mm. In an embodiment, T₃ is about 31 mm. 3 In an embodiment, T₃ is about 2 mm. In an embodiment, T₃ is about 33 mm. In an embodiment, T₃ is about 34 mm. In an embodiment, T₃ is about 35 mm. In an embodiment, T₃ is about 36 mm. In an embodiment, T₃ is about 37 mm. In an embodiment, T₃ is about 38 mm. In an embodiment, T₃ is about 39 mm. In an embodiment, T₃ is about 40 mm.

In an embodiment, the composition has been at rest for about 1 month. In an embodiment, the composition has been at rest for at least 2 weeks. In an embodiment, the composition is stored in a container. In an embodiment, the container is a syringe.

In an embodiment, where after T₃ the composition is resuspended with about 1-10 handshakes. In a preferred embodiment, where after T₃ the composition is resuspended with about 1-5 handshakes. In a more preferred embodiment, after T₃ the composition is resuspended with about 1-3 handshakes. In an embodiment, after T₃ the composition is resuspended with about 1 handshake. In an embodiment, after T₃ the composition is resuspended with about 2 handshakes. In an embodiment, after T₃ the composition is resuspended with about 3 handshakes. In an embodiment, after T₃ the composition is resuspended with about 4 handshakes. 5 handshakes. In an embodiment, after T₃ the composition is resuspended with about 6 handshakes. In an embodiment, after T₃ the composition is resuspended with about 7 handshakes. In an embodiment, after T₃ the composition is resuspended with about 8 handshakes. In an embodiment, after T₃ the composition is resuspended with about 9 handshakes. In an embodiment, after T₃ the composition is resuspended with about 10 handshakes. In an embodiment, the composition comprises the formulation previously described.

In an embodiment, the present invention provides a composition including at least 25 different glycoconjugates and insoluble aluminum phosphate adjuvant, wherein at time T₀ substantially all of the at least 25 different glycoconjugates are dissolved in a liquid phase or adsorbed to the insoluble aluminum phosphate adjuvant as fully dispersed liquid suspension and the at least 25 different glycoconjugates are at a concentration C₀ in the liquid phase; at time T₁ a portion of the at least 25 different glycoconjugates adsorbed to the insoluble adjuvant sediment from the liquid phase to form a sediment and the at least 25 different glycoconjugates are at a concentration C₁ in the liquid phase; at time T₂ additional portion of the at least 25 different glycoconjugates adsorbed to the insoluble adjuvant sediment from the liquid phase to form a sediment and the at least 25 different glycoconjugates are at a concentration C₂ in the liquid phase; and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the height of a flat-bottomed cylindrical glass sample cell, collecting precipitate data every 20 μm.

In one embodiment, the composition includes at least 21 different glycoconjugates. In one embodiment, the composition includes at least 22 different glycoconjugates. In one embodiment, the composition includes at least 23 different glycoconjugates. In one embodiment, the composition includes at least 24 different glycoconjugates. In one embodiment, the composition includes at least 25 different glycoconjugates. In one embodiment, the composition includes at least 26 different glycoconjugates. In one embodiment, the composition includes at least 27 different glycoconjugates. In one embodiment, the composition includes at least 28 different glycoconjugates. In one embodiment, the composition includes at least 29 different glycoconjugates. In one embodiment, the composition includes at least 30 different glycoconjugates. In one embodiment, the composition includes at least 31 different glycoconjugates. In one embodiment, the composition includes at least 32 different includes at least glycoconjugates. In one embodiment, the composition 33 different glycoconjugates. In one embodiment, the composition includes at least 34 different glycoconjugates. In one embodiment, the composition includes at least 35 different glycoconjugates.

In an embodiment, T₀ is 0 hour. In an embodiment, T₁ is about 0.01 hours to 4 hours after the sample reaches 45% clarification at the meniscus. In an embodiment, T₁ is about 1 hour to 2 hours after the sample reaches 45% clarification at the meniscus. In a preferred embodiment, T₁ is about 0.01 hours to 4 hours after the sample reaches 45% clarification at the meniscus. In an embodiment, T₁ is about 0.1 hour after the sample reaches 45% clarification at the meniscus. In an embodiment, T₁ is about 0.2 hour after the sample reaches 45% clarification at the meniscus. In an embodiment, T₁ is about 0.3 hour. In an embodiment, T₁ is about 0.4 hour after the sample reaches 45% clarification at the meniscus. In an embodiment, T₁ is about 0.5 hour after the sample reaches 45% clarification at the meniscus. In an embodiment, T₁ is about 0.6 hour after the sample reaches 45% clarification at the meniscus. In an embodiment, T₁ is about 0.7 hour after the sample reaches 45% clarification at the meniscus. In an embodiment, T₁ is about 0.8 hour after the sample reaches 45% clarification at the meniscus. In an embodiment, T₁ is about 0.9 hour after the sample reaches 45% clarification at the meniscus. In an embodiment, T₁ is about 1.0 hour after the sample reaches 45% clarification at the meniscus. In an embodiment, T₁ is about 1.1 hours after the sample reaches 45% clarification at the meniscus. In an embodiment, T₁ is about 1.2 hours after the sample reaches 45% clarification at the meniscus. In an embodiment, T₁ is about 1.3 hours after the sample reaches 45% clarification at the meniscus. In an embodiment, T₁ is about 1.4 hours after the sample reaches 45% clarification at the meniscus. In an embodiment, T₁ is about 1.5 hours after the sample reaches 45% clarification at the meniscus. In an embodiment, T₁ is about 1.6 hours after the sample reaches 45% clarification at the meniscus. In an embodiment, T₁ is about 1.7 hours after the sample reaches 45% clarification at the meniscus. In an embodiment, T₁ is about 1.8 hours after the sample reaches 45% clarification at the meniscus. In an embodiment, T₁ is about 1.9 hours after the sample reaches 45% clarification at the meniscus. In an embodiment, T₁ is about 2.0 hours after the sample reaches 45% clarification at the meniscus. In an embodiment, T₁ is about 2.1 hours after the sample reaches 45% clarification at the meniscus. In an embodiment, T₁ is about 2.2 hours after the sample reaches 45% clarification at the meniscus. In an embodiment, T₁ is about 2.3 hours after the sample reaches 45% clarification at the meniscus. In an embodiment, T₁ is about 2.4 hours after the sample reaches 45% clarification at the meniscus. In an embodiment, T₁ is about 2.5 hours after the sample reaches 45% clarification at the meniscus. In an embodiment, T₁ is about 2.6 hours after the sample reaches 45% clarification at the meniscus. In an embodiment, T₁ is about 2.7 hours after the sample reaches 45% clarification at the meniscus. In an embodiment, T₁ is about 2.8 hours after the sample reaches 45% clarification at the meniscus. In an embodiment, T₁ is about 2.9 hours after the sample reaches 45% clarification at the meniscus. In an embodiment, T₁ is about 3.0 hours after the sample reaches 45% clarification at the meniscus. In an embodiment, T₁ is about 3.1 hours after the sample reaches 45% clarification at the meniscus. In an embodiment, T₁ is about 3.2 hours after the sample reaches 45% clarification at the meniscus. In an embodiment, T₁ is about 3.3 hours after the sample reaches 45% clarification at the meniscus. In an embodiment, T₁ is about 3.4 hours after the sample reaches 45% clarification at the meniscus. In an embodiment, T₁ is about 3.5 hours after the sample reaches 45% clarification at the meniscus. In an embodiment, T₁ is about 3.6 hours after the sample reaches 45% clarification at the meniscus. In an embodiment, T₁ is about 3.7 hours after the sample reaches 45% clarification at the meniscus. In an embodiment, T₁ is about 3.8 hours after the sample reaches 45% clarification at the meniscus. In an embodiment, T₁ is about 3.9 hours after the sample reaches 45% clarification at the meniscus. In an embodiment, T₁ is about 4.0 hours after the sample reaches 45% clarification at the meniscus.

In an embodiment, T₂ is about 1 hour to 5 hours after the sample reaches 45% clarification at the meniscus. In a preferred embodiment, T₂ is about 1 to 3 hours after the sample reaches 45% clarification at the meniscus. In a more preferred embodiment, T₂ is about 1 to 2 hours after the sample reaches 45% clarification at the meniscus. In a particular embodiment, T₂ is about 4 hours after the sample reaches 45% clarification at the meniscus. In an embodiment, T₂ is about 1.0 hour after the sample reaches 45% clarification at the meniscus. In an embodiment, T₂ is about 1.1 hours after the sample reaches 45% clarification at the meniscus. In an embodiment, T₂ is about 1.2 hours after the sample reaches 45% clarification at the meniscus. In an embodiment, T₂ is about 1.3 hours after the sample reaches 45% clarification at the meniscus. In an embodiment, T₂ is about 1.4 hours after the sample reaches 45% clarification at the meniscus. In an embodiment, T₂ is about 1.5 hours after the sample reaches 45% clarification at the meniscus. In an embodiment, T₂ is about 1.6 hours after the sample reaches 45% clarification at the meniscus. In an embodiment, T₂ is about 1.7 hours after the sample reaches 45% clarification at the meniscus. In an embodiment, T₂ is about 1.8 hours after the sample reaches 45% clarification at the meniscus. In an embodiment, T₂ is about 1.9 hours after the sample reaches 45% clarification at the meniscus. In an embodiment, T₂ is about 2.0 hours after the sample reaches 45% clarification at the meniscus. In an embodiment, T₂ is about 2.1 hours after the sample reaches 45% clarification at the meniscus. In an embodiment, T₂ is about 2.2 hours after the sample reaches 45% clarification at the meniscus. In an embodiment, T₂ is about 2.3 hours after the sample reaches 45% clarification at the meniscus. In an embodiment, T₂ is about 2.4 hours after the sample reaches 45% clarification at the meniscus. In an embodiment, T₂ is about 2.5 hours after the sample reaches 45% clarification at the meniscus. In an embodiment, T₂ is about 2.6 hours after the sample reaches 45% clarification at the meniscus. In an embodiment, T₂ is about 2.7 hours after the sample reaches 45% clarification at the meniscus. In an embodiment, T₂ is about 2.8 hours after the sample reaches 45% clarification at the meniscus. In an embodiment, T₂ is about 2.9 hours after the sample reaches 45% clarification at the meniscus. In an embodiment, T₂ is about 3.0 hours after the sample reaches 45% clarification at the meniscus. In an embodiment, T₂ is about 3.1 hours after the sample reaches 45% clarification at the meniscus. In an embodiment, T₂ is about 3.2 hours after the sample reaches 45% clarification at the meniscus. In an embodiment, T₂ is about 3.3 hours after the sample reaches 45% clarification at the meniscus. In an embodiment, T₂ is about 3.4 hours after the sample reaches 45% clarification at the meniscus. In an embodiment, T₂ is about 3.6 hour after the sample reaches 45% clarification at the meniscus s. In an embodiment, T₂ is about 3.7 hours after the sample reaches 45% clarification at the meniscus. In an embodiment, T₂ is about 3.8 hours after the sample reaches 45% clarification at the meniscus. In an embodiment, T₂ is about 3.9 hours after the sample reaches 45% clarification at the meniscus. In an embodiment, T₂ is about 4.0 hours after the sample reaches 45% clarification at the meniscus. In an embodiment, T₂ is about 4.1 hours after the sample reaches 45% clarification at the meniscus. In an embodiment, T₂ is about 4.2 hours after the sample reaches 45% clarification at the meniscus. In an embodiment, T₂ is about 4.3 hours after the sample reaches 45% clarification at the meniscus. In an embodiment, T₂ is about 4.4 hours after the sample reaches 45% clarification at the meniscus. In an embodiment, T₂ is about 4.5 hours after the sample reaches 45% clarification at the meniscus. In an embodiment, T₂ is about 4.6 hours after the sample reaches 45% clarification at the meniscus. In an embodiment, T₂ is about 4.7 hours after the sample reaches 45% clarification at the meniscus. In an embodiment, T₂ is about 4.8 hours after the sample reaches 45% clarification at the meniscus. In an embodiment, T₂ is about 4.9 hours after the sample reaches 45% clarification at the meniscus. In an embodiment, T₂ is about 5 hours after the sample reaches 45% clarification at the meniscus.

In an embodiment, C₀ is greater than C₁ and C₂. In an embodiment, C₁ is greater than C₂. In one embodiment, C₀ is greater than C₁ and C₂. In one embodiment, C₁ is greater than C₂.

In an embodiment, C₀ is greater than C₁ and C₂. In an embodiment, C₁ is greater than C₂. In one embodiment, C₀ is greater than C₁ and C₂. In one embodiment, C₁ is greater than C₂. In one embodiment, C₀ is greater than C₁ and C₂. In one embodiment, C₁ is greater than C₂.

In an embodiment, at T₁ peak thickness of the sedimentation front is about 0 mm to 20.0 mm. In a preferred embodiment, at T₁ peak thickness of the sedimentation front is about 1 mm to 10.0 mm. In a preferred embodiment, at T₁ peak thickness of the sedimentation front is about 1 mm to 5.0 mm. In a more preferred embodiment, at T₁ peak thickness of the sedimentation front is at least 2 mm. In an embodiment, at T₁ peak thickness of the sedimentation front is about 0 mm. In an embodiment, at T₁ peak thickness of the sedimentation front is about 0.1 mm. In an embodiment, at T₁ peak thickness of the sedimentation front is about 0.2 mm. In an embodiment, at T₁ peak thickness of the sedimentation front is about 0.3 mm. In an embodiment, at T₁ peak thickness of the sedimentation front is about 0.4 mm. In an embodiment, at T₁ peak thickness of the sedimentation front is about 0.5 mm. In an embodiment, at T₁ peak thickness of the sedimentation front is about 0.6 mm. In an embodiment, at T₁ peak thickness of the sedimentation front is about 0.7 mm. In an embodiment, at T₁ peak thickness of the sedimentation front is about 0.8 mm. In an embodiment, at T₁ peak thickness of the sedimentation front is about 0.9 mm. In an embodiment, at T₁ peak thickness of the sedimentation front is about 1.0 mm. In an embodiment, at T₁ peak thickness of the sedimentation front is about 2.0 mm. In an embodiment, at T₁ peak thickness of the sedimentation front is about 3.0 mm. In an embodiment, at T₁ peak thickness of the sedimentation front is about 4.0 mm. In an embodiment, at T₁ peak thickness of the sedimentation front is about 5.0 mm. In an embodiment, at T₁ peak thickness of the sedimentation front is about 6.0 mm. In an embodiment, at T₁ peak thickness of the sedimentation front is about 7.0 mm. In an embodiment, at T₁ peak thickness of the sedimentation front is about 8.0 mm. In an embodiment, at T₁ peak thickness of the sedimentation front is about 9.0 mm. In an embodiment, at T₁ peak thickness of the sedimentation front is about 10.0 mm. In an embodiment, at T₁ peak thickness of the sedimentation front is about 11.0 mm. In an embodiment, at T₁ peak thickness of the sedimentation front is about 12.0 mm. In an embodiment, at T₁ peak thickness of the sedimentation front is about 13.0 mm. In an embodiment, at T₁ peak thickness of the sedimentation front is about 14.0 mm. In an embodiment, at T₁ peak thickness of the sedimentation front is about 15.0 mm. In an embodiment, at T₁ peak thickness of the sedimentation front is about 16.0 mm. In an embodiment, at T₁ peak thickness of the sedimentation front is about 17.0 mm. In an embodiment, at T₁ peak thickness of the sedimentation front is about 18.0 mm. In an embodiment, at T₁ peak thickness of the sedimentation front is about 19.0 mm. In an embodiment, at T₁ peak thickness of the first solid phase sediment is about 20.0 mm. In an embodiment, at T₁ peak thickness of the sedimentation front is about 20.0 mm.

In an embodiment, at T₂ peak thickness of the sedimentation front is about 2 mm to 25.0 mm. In a preferred embodiment, at T₂ peak thickness of the sedimentation front is about 5 mm to 20.0 mm. In a more preferred embodiment, at T₂ peak thickness of the sedimentation front is about 5 mm to 15.0 mm. In an embodiment, at T₂ peak thickness of the sedimentation front is at least 10 mm. In an embodiment, at T₁ peak thickness of the sedimentation front is about 0 mm. In an embodiment, at T₂ peak thickness of the sedimentation front is at least 1.0 mm. In an embodiment, at T₂ peak thickness of the sedimentation front is at least 2.0 mm. In an embodiment, at T₂ peak thickness of the sedimentation front is at least 3.0 mm. In an embodiment, at T₂ peak thickness of the sedimentation front is at least 4.0 mm. In an embodiment, at T₂ peak thickness of the sedimentation front is at least 5.0 mm. In an embodiment, at T₂ peak thickness of the sedimentation front is at least 6.0 mm. In an embodiment, at T₂ peak thickness of the sedimentation front is at least 7.0 mm. In an embodiment, at T₂ peak thickness of the sedimentation front is at least 8.0 mm. In an embodiment, at T₂ peak thickness of the sedimentation front is at least 9.0 mm. In an embodiment, at T₂ peak thickness of the sedimentation front is at least 10.0 mm. In an embodiment, at T₂ peak thickness of the sedimentation front is at least 11.0 mm. In an embodiment, at T₂ peak thickness of the sedimentation front is at least 12.0 mm. In an embodiment, at T₂ peak thickness of the sedimentation front is at least 13.0 mm. In an embodiment, at T₂ peak thickness of the sedimentation front is at least 14.0 mm. In an embodiment, at T₂ peak thickness of the sedimentation front is at least 15.0 mm. In an embodiment, at T₂ peak thickness of the sedimentation front is at least 16.0 mm. In an embodiment, at T₂ peak thickness of the sedimentation front is at least 17.0 mm. In an embodiment, at T₂ peak thickness of the sedimentation front is at least 18.0 mm. In an embodiment, at T₂ peak thickness of the sedimentation front is at least 19.0 mm. In an embodiment, at T₂ peak thickness of the sedimentation front is at least 20.0 mm. In an embodiment, at T₂ peak thickness of the sedimentation front is at least 21.0 mm. In an embodiment, at T₂ peak thickness of the sedimentation front is at least 22.0 mm. In an embodiment, at T₂ peak thickness of the sedimentation front is at least 23.0 mm. In an embodiment, at T₂ peak thickness of the sedimentation front is at least 24.0 mm. In an embodiment, at T₂ peak thickness of the sedimentation front is at least 25.0 mm.

In an embodiment, the sedimentary velocity of the sedimentation front is less than a peak thickness of 10 mm at about 1 hour after the sample reaches 45% clarification at the meniscus and is greater than a peak thickness of 18 mm at about 4 hours after the sample reaches 45% clarification at the meniscus.

In an embodiment, the invention further includes a time T₃ wherein the insoluble aluminum phosphate adsorbed glycoconjugate sedimentation is at equilibrium with the liquid phase at T₃. In an embodiment, T₃ is about 2 hours to 5 hours after the sample reaches 45% clarification at the meniscus. In a preferred embodiment, T₃ is about 3 hours to 5 hours after the sample reaches 45% clarification at the meniscus. In a more preferred embodiment, T₃ is about 4 hours to 5 hours after the sample reaches 45% clarification at the meniscus. In an embodiment, T₃ is about 2.0 hours after the sample reaches 45% clarification at the meniscus. In an embodiment, T₃ is about 2.1 hours after the sample reaches 45% clarification at the meniscus. In an embodiment, T₃ is about 2.2 hours after the sample reaches 45% clarification at the meniscus. In an embodiment, T₃ is about 2.3 hours after the sample reaches 45% clarification at the meniscus. In an embodiment, T₃ is about 2.4 hours after the sample reaches 45% clarification at the meniscus. In an embodiment, T₃ is about 2.5 hours after the sample reaches 45% clarification at the meniscus. In an embodiment, T₃ is about 2.6 hours after the sample reaches 45% clarification at the meniscus. In an embodiment, T₃ is about 2.7 hours after the sample reaches 45% clarification at the meniscus. In an embodiment, T₃ is about 2.8 hours after the sample reaches 45% clarification at the meniscus. In an embodiment, T₃ is about 2.9 hours after the sample reaches 45% clarification at the meniscus. In an embodiment, T₃ is about 3.0 hours after the sample reaches 45% clarification at the meniscus. In an embodiment, T₃ is about 3.1 hours after the sample reaches 45% clarification at the meniscus. In an embodiment, T₃ is about 3.2 hours after the sample reaches 45% clarification at the meniscus. In an embodiment, T₃ is about 3.3 hours after the sample reaches 45% clarification at the meniscus. In an embodiment, T₃ is about 3.4 hours after the sample reaches 45% clarification at the meniscus. In an embodiment, T₃ is about 3.5 hours after the sample reaches 45% clarification at the meniscus. In an embodiment, T₃ is about 3.6 hours after the sample reaches 45% clarification at the meniscus. In an embodiment, T₃ is about 3.7 hours after the sample reaches 45% clarification at the meniscus. In an embodiment, T₃ is about 3.8 hours after the sample reaches 45% clarification at the meniscus. In an embodiment, T₃ is about 3.9 hours after the sample reaches 45% clarification at the meniscus. In an embodiment, T₃ is about 4.0 hours after the sample reaches 45% clarification at the meniscus. In an embodiment, T₃ is about 4.1 hours after the sample reaches 45% clarification at the meniscus. In an embodiment, T₃ is about 4.2 hours after the sample reaches 45% clarification at the meniscus. In an embodiment, T₃ is about 4.3 hours after the sample reaches 45% clarification at the meniscus. In an embodiment, T₃ is about 4.4 hours after the sample reaches 45% clarification at the meniscus. In an embodiment, T₃ is about 4.5 hours after the sample reaches 45% clarification at the meniscus. In an embodiment, T₃ is about 4.6 hours after the sample reaches 45% clarification at the meniscus. In an embodiment, T₃ is about 4.7 hours after the sample reaches 45% clarification at the meniscus. In an embodiment, T₃ is about 4.8 hours after the sample reaches 45% clarification at the meniscus. In an embodiment, T₃ is about 4.9 hours after the sample reaches 45% clarification at the meniscus. In an embodiment, T₃ is about 5 hours after the sample reaches 45% clarification at the meniscus.

In an embodiment, at T₃ the sedimentation front is about 25 mm to 40 mm. In a preferred embodiment, at T₃ the sedimentation front is about 30 mm to 40 mm. In a more preferred embodiment, at T₃ the sedimentation front is about 35 mm to 40 mm. In an embodiment, T₃ is about 25.0 mm. In an embodiment, T₃ is about 26 mm. In an embodiment, T₃ is about 27 mm. In an embodiment, T₃ is about 28 mm. In an embodiment, T₃ is about 29 mm. In an embodiment, T₃ is about 30 mm. In an embodiment, T₃ is about 31 mm. 3 In an embodiment, T₃ is about 2 mm. In an embodiment, T₃ is about 33 mm. In an embodiment, T₃ is about 34 mm. In an embodiment, T₃ is about 35 mm. In an embodiment, T₃ is about 36 mm. In an embodiment, T₃ is about 37 mm. In an embodiment, T₃ is about 38 mm. In an embodiment, T₃ is about 39 mm. In an embodiment, T₃ is about 40 mm.

In an embodiment, the composition has been at rest for about 1 month. In an embodiment, the composition has been at rest for at least 2 weeks. In an embodiment, the composition is stored in a container. In an embodiment, the container is a syringe.

In an embodiment, where after T₃ the composition is resuspended with about 1-10 handshakes. In a preferred embodiment, where after T₃ the composition is resuspended with about 1-5 handshakes. In a more preferred embodiment, after T₃ the composition is resuspended with about 1-3 handshakes. In an embodiment, after T₃ the composition is resuspended with about 1 handshake. In an embodiment, after T₃ the composition is resuspended with about 2 handshakes. In an embodiment, after T₃ the composition is resuspended with about 3 handshakes. In an embodiment, after T₃ the composition is resuspended with about 4 handshakes. 5 handshakes. In an embodiment, after T₃ the composition is resuspended with about 6 handshakes. In an embodiment, after T₃ the composition is resuspended with about 7 handshakes. In an embodiment, after T₃ the composition is resuspended with about 8 handshakes. In an embodiment, after T₃ the composition is resuspended with about 9 handshakes. In an embodiment, after T₃ the composition is resuspended with about 10 handshakes. In an embodiment, the composition comprises the formulation previously described.

In an embodiment, the present invention provides a liquid filled container including at least 25 different glycoconjugates and insoluble aluminum phosphate adjuvant, wherein: at time To, substantially all of the at least 25 different glycoconjugates are dissolved in a liquid phase or adsorbed to the insoluble aluminum phosphate adjuvant as fully dispersed liquid suspension and the at least 25 different glycoconjugates are at a concentration C₀ in the liquid phase; at time T₁, a portion of the at least 25 different glycoconjugates adsorbed to the insoluble adjuvant sediment from the liquid phase to form a sediment and the at least 25 different glycoconjugates has a concentration C₁ in the liquid phase; at time T₂ additional portion of the at least 25 different glycoconjugates adsorbed to the insoluble adjuvant sediment from the liquid phase to form a sediment and the at least 25 different glycoconjugates are at a concentration C₂ in the liquid phase; and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the thickness of a flat-bottomed cylindrical glass sample cell, collecting precipitate data every 20 μm.

In one embodiment, the container includes at least 21 different glycoconjugates. In one embodiment, the container includes at least 22 different glycoconjugates. In one embodiment, the container includes at least 23 different glycoconjugates. In one embodiment, the container includes at least 24 different glycoconjugates. In one embodiment, the container includes at least 25 different glycoconjugates. In one embodiment, the container includes at least 26 different glycoconjugates. In one embodiment, the container includes at least 27 different glycoconjugates. In one embodiment, the container includes at least 28 different glycoconjugates. In one embodiment, the container includes at least 29 different glycoconjugates. In one embodiment, the container includes at least 30 different glycoconjugates. In one embodiment, the container includes at least 31 different glycoconjugates. In one embodiment, the container includes at least 32 different glycoconjugates. In one embodiment, the container includes at least 33 different glycoconjugates. In one embodiment, the container includes at least 34 different glycoconjugates. In one embodiment, the container includes at least 35 different glycoconjugates.

In an embodiment, T₀ is 0 hour. In an embodiment, T₁ is about 0.01 hours to 4 hours after the sample reaches 45% clarification at the meniscus. In an embodiment, T₁ is about 1 hour to 2 hours after the sample reaches 45% clarification at the meniscus. In a preferred embodiment, T₁ is about 0.01 hours to 4 hours after the sample reaches 45% clarification at the meniscus. In an embodiment, T₁ is about 0.1 hour after the sample reaches 45% clarification at the meniscus. In an embodiment, T₁ is about 0.2 hour after the sample reaches 45% clarification at the meniscus. In an embodiment, T₁ is about 0.3 hour. In an embodiment, T₁ is about 0.4 hour after the sample reaches 45% clarification at the meniscus. In an embodiment, T₁ is about 0.5 hour after the sample reaches 45% clarification at the meniscus. In an embodiment, T₁ is about 0.6 hour after the sample reaches 45% clarification at the meniscus. In an embodiment, T₁ is about 0.7 hour after the sample reaches 45% clarification at the meniscus. In an embodiment, T₁ is about 0.8 hour after the sample reaches 45% clarification at the meniscus. In an embodiment, T₁ is about 0.9 hour after the sample reaches 45% clarification at the meniscus. In an embodiment, T₁ is about 1.0 hour after the sample reaches 45% clarification at the meniscus. In an embodiment, T₁ is about 1.1 hours after the sample reaches 45% clarification at the meniscus. In an embodiment, T₁ is about 1.2 hours after the sample reaches 45% clarification at the meniscus. In an embodiment, T₁ is about 1.3 hours after the sample reaches 45% clarification at the meniscus. In an embodiment, T₁ is about 1.4 hours after the sample reaches 45% clarification at the meniscus. In an embodiment, T₁ is about 1.5 hours after the sample reaches 45% clarification at the meniscus. In an embodiment, T₁ is about 1.6 hours after the sample reaches 45% clarification at the meniscus. In an embodiment, T₁ is about 1.7 hours after the sample reaches 45% clarification at the meniscus. In an embodiment, T₁ is about 1.8 hours after the sample reaches 45% clarification at the meniscus. In an embodiment, T₁ is about 1.9 hours after the sample reaches 45% clarification at the meniscus. In an embodiment, T₁ is about 2.0 hours after the sample reaches 45% clarification at the meniscus. In an embodiment, T₁ is about 2.1 hours after the sample reaches 45% clarification at the meniscus. In an embodiment, T₁ is about 2.2 hours after the sample reaches 45% clarification at the meniscus. In an embodiment, T₁ is about 2.3 hours after the sample reaches 45% clarification at the meniscus. In an embodiment, T₁ is about 2.4 hours after the sample reaches 45% clarification at the meniscus. In an embodiment, T₁ is about 2.5 hours after the sample reaches 45% clarification at the meniscus. In an embodiment, T₁ is about 2.6 hours after the sample reaches 45% clarification at the meniscus. In an embodiment, T₁ is about 2.7 hours after the sample reaches 45% clarification at the meniscus. In an embodiment, T₁ is about 2.8 hours after the sample reaches 45% clarification at the meniscus. In an embodiment, T₁ is about 2.9 hours after the sample reaches 45% clarification at the meniscus. In an embodiment, T₁ is about 3.0 hours after the sample reaches 45% clarification at the meniscus. In an embodiment, T₁ is about 3.1 hours after the sample reaches 45% clarification at the meniscus. In an embodiment, T₁ is about 3.2 hours after the sample reaches 45% clarification at the meniscus. In an embodiment, T₁ is about 3.3 hours after the sample reaches 45% clarification at the meniscus. In an embodiment, T₁ is about 3.4 hours after the sample reaches 45% clarification at the meniscus. In an embodiment, T₁ is about 3.5 hours after the sample reaches 45% clarification at the meniscus. In an embodiment, T₁ is about 3.6 hours after the sample reaches 45% clarification at the meniscus. In an embodiment, T₁ is about 3.7 hours after the sample reaches 45% clarification at the meniscus. In an embodiment, T₁ is about 3.8 hours after the sample reaches 45% clarification at the meniscus. In an embodiment, T₁ is about 3.9 hours after the sample reaches 45% clarification at the meniscus. In an embodiment, T₁ is about 4.0 hours after the sample reaches 45% clarification at the meniscus.

In an embodiment, T₂ is about 1 hour to 5 hours after the sample reaches 45% clarification at the meniscus. In a preferred embodiment, T₂ is about 1 to 3 hours after the sample reaches 45% clarification at the meniscus. In a more preferred embodiment, T₂ is about 1 to 2 hours after the sample reaches 45% clarification at the meniscus. In a particular embodiment, T₂ is about 4 hours after the sample reaches 45% clarification at the meniscus. In an embodiment, T₂ is about 1.0 hour after the sample reaches 45% clarification at the meniscus. In an embodiment, T₂ is about 1.1 hours after the sample reaches 45% clarification at the meniscus. In an embodiment, T₂ is about 1.2 hours after the sample reaches 45% clarification at the meniscus. In an embodiment, T₂ is about 1.3 hours after the sample reaches 45% clarification at the meniscus. In an embodiment, T₂ is about 1.4 hours after the sample reaches 45% clarification at the meniscus. In an embodiment, T₂ is about 1.5 hours after the sample reaches 45% clarification at the meniscus. In an embodiment, T₂ is about 1.6 hours after the sample reaches 45% clarification at the meniscus. In an embodiment, T₂ is about 1.7 hours after the sample reaches 45% clarification at the meniscus. In an embodiment, T₂ is about 1.8 hours after the sample reaches 45% clarification at the meniscus. In an embodiment, T₂ is about 1.9 hours after the sample reaches 45% clarification at the meniscus. In an embodiment, T₂ is about 2.0 hours after the sample reaches 45% clarification at the meniscus. In an embodiment, T₂ is about 2.1 hours after the sample reaches 45% clarification at the meniscus. In an embodiment, T₂ is about 2.2 hours after the sample reaches 45% clarification at the meniscus. In an embodiment, T₂ is about 2.3 hours after the sample reaches 45% clarification at the meniscus. In an embodiment, T₂ is about 2.4 hours after the sample reaches 45% clarification at the meniscus. In an embodiment, T₂ is about 2.5 hours after the sample reaches 45% clarification at the meniscus. In an embodiment, T₂ is about 2.6 hours after the sample reaches 45% clarification at the meniscus. In an embodiment, T₂ is about 2.7 hours after the sample reaches 45% clarification at the meniscus. In an embodiment, T₂ is about 2.8 hours after the sample reaches 45% clarification at the meniscus. In an embodiment, T₂ is about 2.9 hours after the sample reaches 45% clarification at the meniscus. In an embodiment, T₂ is about 3.0 hours after the sample reaches 45% clarification at the meniscus. In an embodiment, T₂ is about 3.1 hours after the sample reaches 45% clarification at the meniscus. In an embodiment, T₂ is about 3.2 hours after the sample reaches 45% clarification at the meniscus. In an embodiment, T₂ is about 3.3 hours after the sample reaches 45% clarification at the meniscus. In an embodiment, T₂ is about 3.4 hours after the sample reaches 45% clarification at the meniscus. In an embodiment, T₂ is about 3.6 hour after the sample reaches 45% clarification at the meniscus s. In an embodiment, T₂ is about 3.7 hours after the sample reaches 45% clarification at the meniscus. In an embodiment, T₂ is about 3.8 hours after the sample reaches 45% clarification at the meniscus. In an embodiment, T₂ is about 3.9 hours after the sample reaches 45% clarification at the meniscus. In an embodiment, T₂ is about 4.0 hours after the sample reaches 45% clarification at the meniscus. In an embodiment, T₂ is about 4.1 hours after the sample reaches 45% clarification at the meniscus. In an embodiment, T₂ is about 4.2 hours after the sample reaches 45% clarification at the meniscus. In an embodiment, T₂ is about 4.3 hours after the sample reaches 45% clarification at the meniscus. In an embodiment, T₂ is about 4.4 hours after the sample reaches 45% clarification at the meniscus. In an embodiment, T₂ is about 4.5 hours after the sample reaches 45% clarification at the meniscus. In an embodiment, T₂ is about 4.6 hours after the sample reaches 45% clarification at the meniscus. In an embodiment, T₂ is about 4.7 hours after the sample reaches 45% clarification at the meniscus. In an embodiment, T₂ is about 4.8 hours after the sample reaches 45% clarification at the meniscus. In an embodiment, T₂ is about 4.9 hours after the sample reaches 45% clarification at the meniscus. In an embodiment, T₂ is about 5 hours after the sample reaches 45% clarification at the meniscus.

In an embodiment, C₀ is greater than C₁ and C₂. In an embodiment, C₁ is greater than C₂. In one embodiment, C₀ is greater than C₁ and C₂. In one embodiment, C₁ is greater than C₂.

In an embodiment, C₀ is greater than C₁ and C₂. In an embodiment, C₁ is greater than C₂. In one embodiment, C₀ is greater than C₁ and C₂. In one embodiment, C₁ is greater than C₂. In one embodiment, C₀ is greater than C₁ and C₂. In one embodiment, C₁ is greater than C₂.

In an embodiment, at T₁ peak thickness of the sedimentation front is about 0 mm to 20.0 mm. In a preferred embodiment, at T₁ peak thickness of the sedimentation front is about 1 mm to 10.0 mm. In a more preferred embodiment, at T₁ peak thickness of the sedimentation front is about 1 mm to 5.0 mm. In a particular embodiment, at T₁ peak thickness of the sedimentation front is at least 2 mm. In an embodiment, at T₁ peak thickness of the sedimentation front is about 0 mm. In an embodiment, at T₁ peak thickness of the sedimentation front is about 0.1 mm. In an embodiment, at T₁ peak thickness of the sedimentation front is about 0.2 mm. In an embodiment, at T₁ peak thickness of the sedimentation front is about 0.3 mm. In an embodiment, at T₁ peak thickness of the sedimentation front is about 0.4 mm. In an embodiment, at T₁ peak thickness of the sedimentation front is about 0.5 mm. In an embodiment, at T₁ peak thickness of the sedimentation front is about 0.6 mm. In an embodiment, at T₁ peak thickness of the sedimentation front is about 0.7 mm. In an embodiment, at T₁ peak thickness of the sedimentation front is about 0.8 mm. In an embodiment, at T₁ peak thickness of the sedimentation front is about 0.9 mm. In an embodiment, at T₁ peak thickness of the sedimentation front is about 1.0 mm. In an embodiment, at T₁ peak thickness of the sedimentation front is about 2.0 mm. In an embodiment, at T₁ peak thickness of the sedimentation front is about 3.0 mm. In an embodiment, at T₁ peak thickness of the sedimentation front is about 4.0 mm. In an embodiment, at T₁ peak thickness of the sedimentation front is about 5.0 mm. In an embodiment, at T₁ peak thickness of the sedimentation front is about 6.0 mm. In an embodiment, at T₁ peak thickness of the sedimentation front is about 7.0 mm. In an embodiment, at T₁ peak thickness of the sedimentation front is about 8.0 mm. In an embodiment, at T₁ peak thickness of the sedimentation front is about 9.0 mm. In an embodiment, at T₁ peak thickness of the sedimentation front is about 10.0 mm. In an embodiment, at T₁ peak thickness of the sedimentation front is about 11.0 mm. In an embodiment, at T₁ peak thickness of the sedimentation front is about 12.0 mm. In an embodiment, at T₁ peak thickness of the sedimentation front is about 13.0 mm. In an embodiment, at T₁ peak thickness of the sedimentation front is about 14.0 mm. In an embodiment, at T₁ peak thickness of the sedimentation front is about 15.0 mm. In an embodiment, at T₁ peak thickness of the sedimentation front is about 16.0 mm. In an embodiment, at T₁ peak thickness of the sedimentation front is about 17.0 mm. In an embodiment, at T₁ peak thickness of the sedimentation front is about 18.0 mm. In an embodiment, at T₁ peak thickness of the sedimentation front is about 19.0 mm. In an embodiment, at T₁ peak thickness of the sedimentation front is about 20.0 mm.

In an embodiment, at T₂ peak thickness of the sedimentation front is about 2 mm to 25.0 mm. In a preferred embodiment, at T₂ peak thickness of the sedimentation front is about 5 mm to 20.0 mm. In a more preferred embodiment, at T₂ peak thickness of the sedimentation front is about 5 mm to 15.0 mm. In a particular embodiment, at T₂ peak thickness of the sedimentation front is at least 10 mm. In an embodiment, at T₁ peak thickness of the sedimentation front is about 0 mm. In an embodiment, at T₂ peak thickness of the sedimentation front is at least 1.0 mm. In an embodiment, at T₂ peak thickness of the sedimentation front is at least 2.0 mm. In an embodiment, at T₂ peak thickness of the sedimentation front is at least 3.0 mm. In an embodiment, at T₂ peak thickness of the sedimentation front is at least 4.0 mm. In an embodiment, at T₂ peak thickness of the sedimentation front is at least 5.0 mm. In an embodiment, at T₂ peak thickness of the sedimentation front is at least 6.0 mm. In an embodiment, at T₂ peak thickness of the sedimentation front is at least 7.0 mm. In an embodiment, at T₂ peak thickness of the sedimentation front is at least 8.0 mm. In an embodiment, at T₂ peak thickness of the sedimentation front is at least 9.0 mm. In an embodiment, at T₂ peak thickness of the sedimentation front is at least 10.0 mm. In an embodiment, at T₂ peak thickness of the sedimentation front is at least 11.0 mm. In an embodiment, at T₂ peak thickness of the sedimentation front is at least 12.0 mm. In an embodiment, at T₂ peak thickness of the sedimentation front is at least 13.0 mm. In an embodiment, at T₂ peak thickness of the sedimentation front is at least 14.0 mm. In an embodiment, at T₂ peak thickness of the sedimentation front is at least 15.0 mm. In an embodiment, at T₂ peak thickness of the sedimentation front is at least 16.0 mm. In an embodiment, at T₂ peak thickness of the sedimentation front is at least 17.0 mm. In an embodiment, at T₂ peak thickness of the sedimentation front is at least 18.0 mm. In an embodiment, at T₂ peak thickness of the sedimentation front is at least 19.0 mm. In an embodiment, at T₂ peak thickness of the sedimentation front is at least 20.0 mm. In an embodiment, at T₂ peak thickness of the sedimentation front is at least 21.0 mm. In an embodiment, at T₂ peak thickness of the sedimentation front is at least 22.0 mm. In an embodiment, at T₂ peak thickness of the sedimentation front is at least 23.0 mm. In an embodiment, at T₂ peak thickness of the sedimentation front is at least 24.0 mm. In an embodiment, at T₂ peak thickness of the sedimentation front is at least 25.0 mm.

In an embodiment, the sedimentary velocity of the sedimentation front is less than a peak thickness of 10 mm at about 1 hour after the sample reaches 45% clarification at the meniscus and is greater than a peak thickness of 18 mm at about 4 hours after the sample reaches 45% clarification at the meniscus.

In an embodiment, the invention further includes a time T₃ wherein the insoluble aluminum phosphate adsorbed glycoconjugate sedimentation is at equilibrium with the liquid phase at T₃. In an embodiment, T₃ is about 2 hours to 5 hours after the sample reaches 45% clarification at the meniscus. In a preferred embodiment, T₃ is about 3 hours to 5 hours after the sample reaches 45% clarification at the meniscus. In a more preferred embodiment, T₃ is about 4 hours to 5 hours after the sample reaches 45% clarification at the meniscus. In an embodiment, T₃ is about 2.0 hours after the sample reaches 45% clarification at the meniscus. In an embodiment, T₃ is about 2.1 hours after the sample reaches 45% clarification at the meniscus. In an embodiment, T₃ is about 2.2 hours after the sample reaches 45% clarification at the meniscus. In an embodiment, T₃ is about 2.3 hours after the sample reaches 45% clarification at the meniscus. In an embodiment, T₃ is about 2.4 hours after the sample reaches 45% clarification at the meniscus. In an embodiment, T₃ is about 2.5 hours after the sample reaches 45% clarification at the meniscus. In an embodiment, T₃ is about 2.6 hours after the sample reaches 45% clarification at the meniscus. In an embodiment, T₃ is about 2.7 hours after the sample reaches 45% clarification at the meniscus. In an embodiment, T₃ is about 2.8 hours after the sample reaches 45% clarification at the meniscus. In an embodiment, T₃ is about 2.9 hours after the sample reaches 45% clarification at the meniscus. In an embodiment, T₃ is about 3.0 hours after the sample reaches 45% clarification at the meniscus. In an embodiment, T₃ is about 3.1 hours after the sample reaches 45% clarification at the meniscus. In an embodiment, T₃ is about 3.2 hours after the sample reaches 45% clarification at the meniscus. In an embodiment, T₃ is about 3.3 hours after the sample reaches 45% clarification at the meniscus. In an embodiment, T₃ is about 3.4 hours after the sample reaches 45% clarification at the meniscus. In an embodiment, T₃ is about 3.5 hours after the sample reaches 45% clarification at the meniscus. In an embodiment, T₃ is about 3.6 hours after the sample reaches 45% clarification at the meniscus. In an embodiment, T₃ is about 3.7 hours after the sample reaches 45% clarification at the meniscus. In an embodiment, T₃ is about 3.8 hours after the sample reaches 45% clarification at the meniscus. In an embodiment, T₃ is about 3.9 hours after the sample reaches 45% clarification at the meniscus. In an embodiment, T₃ is about 4.0 hours after the sample reaches 45% clarification at the meniscus. In an embodiment, T₃ is about 4.1 hours after the sample reaches 45% clarification at the meniscus. In an embodiment, T₃ is about 4.2 hours after the sample reaches 45% clarification at the meniscus. In an embodiment, T₃ is about 4.3 hours after the sample reaches 45% clarification at the meniscus. In an embodiment, T₃ is about 4.4 hours after the sample reaches 45% clarification at the meniscus. In an embodiment, T₃ is about 4.5 hours after the sample reaches 45% clarification at the meniscus. In an embodiment, T₃ is about 4.6 hours after the sample reaches 45% clarification at the meniscus. In an embodiment, T₃ is about 4.7 hours after the sample reaches 45% clarification at the meniscus. In an embodiment, T₃ is about 4.8 hours after the sample reaches 45% clarification at the meniscus. In an embodiment, T₃ is about 4.9 hours after the sample reaches 45% clarification at the meniscus. In an embodiment, T₃ is about 5 hours after the sample reaches 45% clarification at the meniscus.

In an embodiment, at T₃ the sedimentation front is about 25 mm to 40 mm. In a preferred embodiment, at T₃ the sedimentation front is about 30 mm to 40 mm. In a more preferred embodiment, at T₃ the sedimentation front is about 35 mm to 40 mm. In an embodiment, T₃ is about 25.0 mm. In an embodiment, T₃ is about 26 mm. In an embodiment, T₃ is about 27 mm. In an embodiment, T₃ is about 28 mm. In an embodiment, T₃ is about 29 mm. In an embodiment, T₃ is about 30 mm. In an embodiment, T₃ is about 31 mm. 3 In an embodiment, T₃ is about 2 mm. In an embodiment, T₃ is about 33 mm. In an embodiment, T₃ is about 34 mm. In an embodiment, T₃ is about 35 mm. In an embodiment, T₃ is about 36 mm. In an embodiment, T₃ is about 37 mm. In an embodiment, T₃ is about 38 mm. In an embodiment, T₃ is about 39 mm. In an embodiment, T₃ is about 40 mm.

In an embodiment, the composition has been at rest for about 1 month. In an embodiment, the composition has been at rest for at least 2 weeks. In an embodiment, the composition is stored in a container. In an embodiment, the container is a syringe.

In an embodiment, where after T₃ the composition is resuspended with about 1-10 handshakes. In a preferred embodiment, where after T₃ the composition is resuspended with about 1-5 handshakes. In a more preferred embodiment, after T₃ the composition is resuspended with about 1-3 handshakes. In an embodiment, after T₃ the composition is resuspended with about 1 handshake. In an embodiment, after T₃ the composition is resuspended with about 2 handshakes. In an embodiment, after T₃ the composition is resuspended with about 3 handshakes. In an embodiment, after T₃ the composition is resuspended with about 4 handshakes. 5 handshakes. In an embodiment, after T₃ the composition is resuspended with about 6 handshakes. In an embodiment, after T₃ the composition is resuspended with about 7 handshakes. In an embodiment, after T₃ the composition is resuspended with about 8 handshakes. In an embodiment, after T₃ the composition is resuspended with about 9 handshakes. In an embodiment, after T₃ the composition is resuspended with about 10 handshakes. In an embodiment, the composition comprises the formulation previously described.

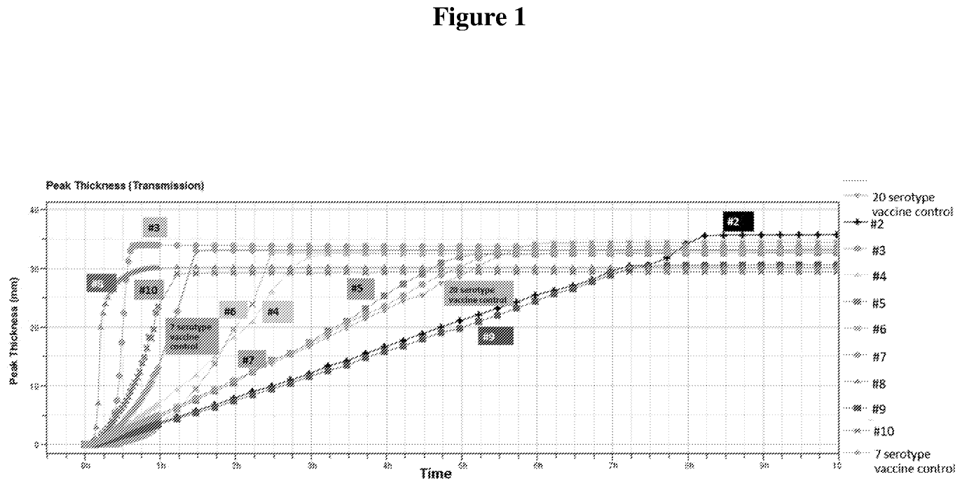

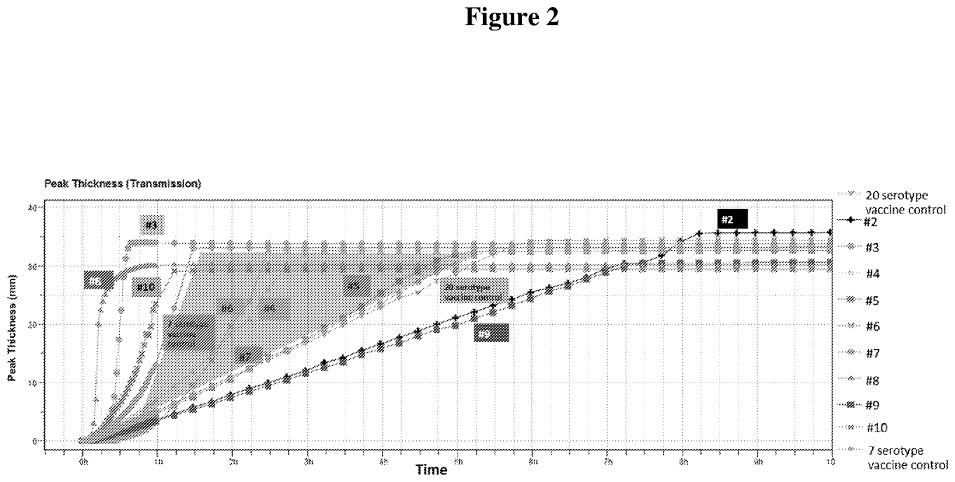

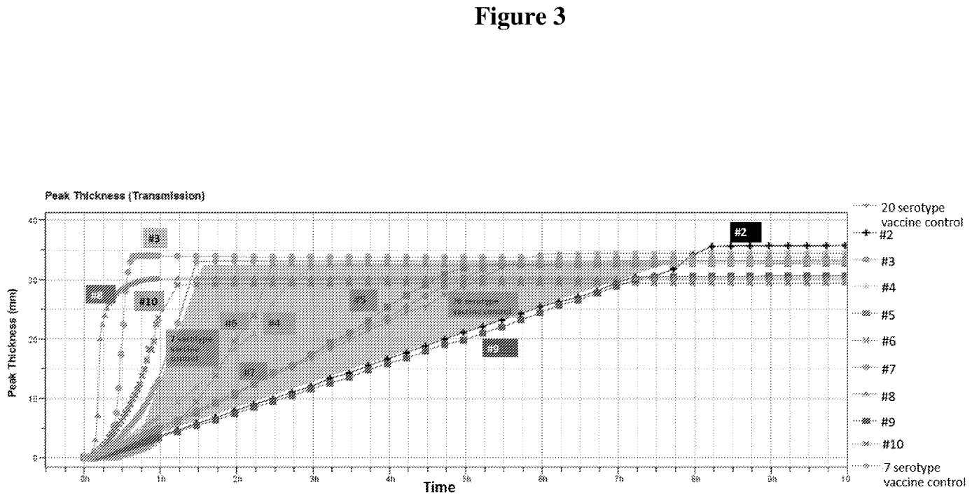

FIGS. 1-3 provide sedimentation curves for comparative formulations and formulations of the present invention. In one embodiment, the sedimentation velocity of the first solid phase sediment is less than the sedimentation rate of the second solid phase sediment. The formulations of the present invention sediment at a suitable rate to permit manufacture, resuspension and use. In particular embodiments, formulations of the present invention, have sedimentation velocities that are faster than the 20 serotypes control formulation. In certain embodiments, formulations of the present invention, have sedimentation velocities that fall between the sedimentation curve of the seven serotypes control formulation and the 20 serotypes control formulation (FIG. 1 and shaded area of FIG. 2). In certain embodiments, formulations of the present invention, have sedimentation velocities that fall between the sedimentation curve of the seven serotypes control formulation and the 25 serotypes control formulation (FIG. 1 and shaded area of FIG. 3). In certain embodiments, formulations of the present invention, have sedimentation velocities that fall within the shaded area of FIG. 2 or FIG. 3. exemplified by a number of matrices detailed in Table 1 below.

In one embodiment, the present invention provides a syringe filled with any of the vaccine formulations disclosed herein. In certain embodiments, the syringe is siliconized and/or is made of glass.

A typical dose of the vaccine formulations of the invention for injection has a volume of 0.1 mL to 2 mL, more preferably 0.2 mL to 1 mL, even more preferably a volume of about 0.5 mL.

Therefore, the container or syringe as defined above is filed with a volume of 0.1 mL to 2 mL, more preferably 0.2 mL to 1 mL, even more preferably a volume of about 0.5 mL of any of the vaccine formulations defined herein.

### Adjuvants

In some embodiments, the vaccine formulations disclosed herein may further comprise at least one, two or three adjuvants. In some embodiments, the vaccine formulations disclosed herein may further comprise at least one adjuvant. In some embodiments, the vaccine formulations disclosed herein may further comprise one adjuvant. In some embodiments, the vaccine formulations disclosed herein may further comprise two adjuvants. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the formulations disclosed herein comprise aluminum salts (alum) as adjuvant (e.g., aluminum phosphate, aluminum sulfate or aluminum hydroxide). In a preferred embodiment, the vaccine formulations disclosed herein comprise aluminum phosphate or aluminum hydroxide as adjuvant. In a preferred embodiment, the vaccine formulations disclosed herein comprise aluminum phosphate as adjuvant.

Further exemplary adjuvants to enhance effectiveness of the vaccine formulations as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally +a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc. In an embodiment of the present invention, the vaccine formulations as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

In an embodiment of the present invention, the vaccine formulations as disclosed herein comprise any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

In an embodiment of the present invention, the vaccine formulations as disclosed herein comprise an A class CpG oligonucleotide. Preferably, the “A class” CpG oligonucleotide of the invention has the following nucleic acid sequence: 5′ GGGGACGACGTCGTGGGGGGG 3′ (SEQ ID NO: 1). Some non-limiting examples of A-Class oligonucleotides include: 5′ G*G*G_G_A_C_G_A_C_G_T_C_G_T_G_G*G*G*G*G*G 3′ (SEQ ID NO: 2); wherein “*” refers to a phosphorothioate bond and “_” refers to a phosphodiester bond.

In an embodiment of the present invention, the vaccine formulations as disclosed herein comprise a B class CpG Oligonucleotide. In one embodiment, the CpG oligonucleotide for use in the present invention is a B class CpG oligonucleotide represented by at least the formula:
5′ X1X2CGX3X4 3′, wherein X1, X2, X3, and X4 are nucleotides. In one embodiment, X2 is adenine, guanine, or thymine. In another embodiment, X3 is cytosine, adenine, or thymine.

The B class CpG oligonucleotide sequences of the invention are those broadly described above as well as disclosed in WO 96/02555, WO 98/18810 and U.S. Pat. Nos. 6,194,388; 6,207,646; 6,214,806; 6,218,371; 6,239,116 and 6,339,068. Exemplary sequences include but are not limited to those disclosed in these latter applications and patents.

In an embodiment, the “B class” CpG oligonucleotide of the invention has the following nucleic acid sequence:

(SEQ ID NO: 3)
5′ TCGTCGTTTTTCGGTGCTTTT 3′,
or
(SEQ ID NO: 4)
5′ TCGTCGTTTTTCGGTCGTTTT 3′,
or
(SEQ ID NO: 5)
5′ TCGTCGTTTTGTCGTTTTGTCGTT 3′,
or
(SEQ ID NO: 6)
5′ TCGTCGTTTCGTCGTTTTGTCGTT 3′,
or
(SEQ ID NO: 7)
5′ TCGTCGTTTTGTCGTTTTTTTCGA 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

Some non-limiting examples of B-Class oligonucleotides include:

(SEQ ID NO: 8)
5′ T*C*G*T*C*G*T*T*T*T*T*C*G*G*T*G*C*T*T*T*T 3′,
or
(SEQ ID NO: 9)
5′ T*C*G*T*C*G*T*T*T*T*T*C*G*G*T*C*G*T*T*T*T 3′,
or
(SEQ ID NO: 10)
5′ T*C*G*T*C*G*T*T*T*T*G*T*C*G*T*T*T*T*G*T*C*G*T*
T 3′,
or
(SEQ ID NO: 11)
5′ T*C*G*T*C*G*T*T*T*C*G*T*C*G*T*T*T*T*G*T*C*G*T*
T 3′
or
(SEQ ID NO: 12)
5′ T*C*G*T*C*G*T*T*T*T*G*T*C*G*T*T*T*T*T*T*T*C*G*
A 3′,.

wherein “*” refers to a phosphorothioate bond.

In an embodiment of the present invention, the vaccine formulations as disclosed herein comprise a C class CpG Oligonucleotide. In an embodiment, the “C class” CpG oligonucleotides of the invention have the following nucleic acid sequence:

(SEQ ID NO: 13)
5′ TCGCGTCGTTCGGCGCGCGCCG 3′,
or
(SEQ ID NO: 14)
5′ TCGTCGACGTTCGGCGCGCGCCG 3′,
or
(SEQ ID NO: 15)
5′ TCGGACGTTCGGCGCGCGCCG 3′,
or
(SEQ ID NO: 16)
5′ TCGGACGTTCGGCGCGCCG 3′,
or
(SEQ ID NO: 17)
5′ TCGCGTCGTTCGGCGCGCCG 3′,
or
(SEQ ID NO: 18)
5′ TCGACGTTCGGCGCGCGCCG 3′,
or
(SEQ ID NO: 19)
5′ TCGACGTTCGGCGCGCCG 3′,
or
(SEQ ID NO: 20)
5′ TCGCGTCGTTCGGCGCCG 3′,
or
(SEQ ID NO: 21)
5′ TCGCGACGTTCGGCGCGCGCCG 3′,
or
(SEQ ID NO: 22)
5′ TCGTCGTTTTCGGCGCGCGCCG 3′,
or
(SEQ ID NO: 23)
5′ TCGTCGTTTTCGGCGGCCGCCG 3′,
or
(SEQ ID NO: 24)
5′ TCGTCGTTTTACGGCGCCGTGCCG 3′,
or
(SEQ ID NO: 25)
5′ TCGTCGTTTTCGGCGCGCGCCGT 3′.

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide.

Some non-limiting examples of C-Class oligonucleotides include:

(SEQ ID NO: 26)
5′ T*C_G*C_G*T*C_G*T*T*C_G*G*C*G*C_G*C*G*C*C*G 3′,
or
(SEQ ID NO: 27)
5′ T*C_G*T*C_G*A*C_G*T*T*C_G*G*C*G*C_G*C*G*C*C*G
3′,
or
(SEQ ID NO: 28)
5′ T*C_G*G*A*C_G*T*T*C_G*G*C*G*C_G*C*G*C*C*G 3′,
or
(SEQ ID NO: 29)
5′ T*C_G*G*A*C_G*T*T*C_G*G*C*G*C*G*C*C*G 3′,
or
(SEQ ID NO: 30)
5′ T*C_G*C_G*T*C_G*T*T*C_G*G*C*G*C*G*C*C*G 3′,
or
(SEQ ID NO: 31)
5′ T*C_G*A*C_G*T*T*C_G*G*C*G*C_G*C*G*C*C*G 3′,
or
(SEQ ID NO: 32)
5′ T*C_G*A*C_G*T*T*C_G*G*C*G*C*G*C*C*G 3′,
or
(SEQ ID NO: 33)
5′ T*C_G*C_G*T*C_G*T*T*C_G*G*C*G*C*C*G 3′,
or
(SEQ ID NO: 34)
5′ T*C_G*C_G*A*C_G*T*T*C_G*G*C*G*C_G*C*G*C*C*G 3′,
or
(SEQ ID NO: 35)
5′ T*C*G*T*C*G*T*T*T*T*C*G*G*C*G*C*G*C*G*C*C*G 3′,
or
(SEQ ID NO: 36)
5′ T*C*G*T*C*G*T*T*T*T*C*G*G*C*G*G*C*C*G*C*C*G 3′,
or
(SEQ ID NO: 37)
5′ T*C*G*T*C_G*T*T*T*T*A*C_G*G*C*G*C*C_G*T*G*C*C*
G 3′,
or
(SEQ ID NO: 38)
5′ T*C_G*T*C*G*T*T*T*T*C*G*G*C*G*C*G*C*G*C*C*G*T
3′

wherein “*” refers to a phosphorothioate bond and “_” refers to a phosphodiester bond.

In any of these sequences, an ethyl-uridine or a halogen may substitute for the 5′ T; examples of halogen substitutions include but are not limited to bromo-uridine or iodo-uridine substitutions.

In an embodiment of the present invention, the vaccine formulations as disclosed herein comprise a P class CpG Oligonucleotide. In an embodiment, the CpG oligonucleotide for use in the present invention is a P class CpG oligonucleotide containing a 5′ TLR activation domain and at least two palindromic regions, one palindromic region being a 5′ palindromic region of at least 6 nucleotides in length and connected to a 3′ palindromic region of at least 8 nucleotides in length either directly or through a spacer, wherein the oligonucleotide includes at least one YpR dinucleotide. In an embodiment, said oligonucleotide is not T*C_G*T*C_G*A*C_G*T*T*C_G*G*C*G*C_G*C*G*C*C*G (SEQ ID NO: 27). In one embodiment the P class CpG oligonucleotide includes at least one unmethylated CpG dinucleotide. In another embodiment the TLR activation domain is TCG, TTCG, TTTCG, TYpR, TTYpR, TTTYpR, UCG, UUCG, UUUCG, TTT, or TTTT. In yet another embodiment the TLR activation domain is within the 5′ palindromic region. In another embodiment the TLR activation domain is immediately 5′ to the 5′ palindromic region.

In an embodiment, the “P class” CpG oligonucleotides of the invention have the following nucleic acid sequence: 5′ TCGTCGACGATCGGCGCGCGCCG 3′ (SEQ ID NO: 39).

In said sequences, all of the linkages may be all phosphorothioate bonds. In another embodiment, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

A non-limiting example of P-Class oligonucleotides include:

(SEQ ID NO: 40)
5′ T*C_G*T*C_G*A*C_G*A*T*C_G*G*C*G*C_G*C*G*C*C*G
3′

wherein “*” refers to a phosphorothioate bond and “_” refers to a phosphodiester bond.

In one embodiment the oligonucleotide includes at least one phosphorothioate linkage. In another embodiment all internucleotide linkages of the oligonucleotide are phosphorothioate linkages. In another embodiment the oligonucleotide includes at least one phosphodiester-like linkage. In another embodiment the phosphodiester-like linkage is a phosphodiester linkage. In another embodiment a lipophilic group is conjugated to the oligonucleotide. In one embodiment the lipophilic group is cholesterol.

In an embodiment, all the internucleotide linkages of the CpG oligonucleotides disclosed herein are phosphodiester bonds (“soft” oligonucleotides, as described in WO 2007/026190). In another embodiment, CpG oligonucleotides of the invention are rendered resistant to degradation (e.g., are stabilized). A “stabilized oligonucleotide” refers to an oligonucleotide that is relatively resistant to in vivo degradation (e.g., via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases.

The immunostimulatory oligonucleotides may have a chimeric backbone, which have combinations of phosphodiester and phosphorothioate linkages. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. When the phosphodiester linkage is preferentially located within the CpG motif such molecules are called “semi-soft” as described in WO 2007/026190.

Other modified oligonucleotides include combinations of phosphodiester, phosphorothioate, methylphosphonate, methylphosphorothioate, phosphorodithioate, and/or p-ethoxy linkages.

Mixed backbone modified ODN may be synthesized as described in WO 2007/026190.

The size of the CpG oligonucleotide (i.e., the number of nucleotide residues along the length of the oligonucleotide) also may contribute to the stimulatory activity of the oligonucleotide. For facilitating uptake into cells, CpG oligonucleotide of the invention preferably have a minimum length of 6 nucleotide residues. Oligonucleotides of any size greater than 6 nucleotides (even many kb long) are capable of inducing an immune response if sufficient immunostimulatory motifs are present, because larger oligonucleotides are degraded inside cells. In certain embodiments, the CpG oligonucleotides are 6 to 100 nucleotides long, preferentially 8 to 30 nucleotides long. In important embodiments, nucleic acids and oligonucleotides of the invention are not plasmids or expression vectors.

In an embodiment, the CpG oligonucleotide disclosed herein comprise substitutions or modifications, such as in the bases and/or sugars as described at paragraphs 134 to 147 of WO 2007/026190.

In an embodiment, the CpG oligonucleotide of the present invention is chemically modified. Examples of chemical modifications are known to the skilled person and are described, for example in Uhlmann et al. (1990) Chem. Rev. 90:543; S. Agrawal, Ed., Humana Press, Totowa, USA 1993; Crooke et al. (1996) Annu. Rev. Pharmacol. Toxicol. 36:107-129; and Hunziker et al. (1995) Mod. Synth. Methods 7:331-417. An oligonucleotide according to the invention may have one or more modifications, wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

In some embodiments of the invention, CpG-containing nucleic acids might be simply mixed with immunogenic carriers according to methods known to those skilled in the art (see, e.g., WO 03/024480).

In a particular embodiment of the present invention, any of the vaccine formulations disclosed herein comprise from 2 μg to 100 mg of CpG oligonucleotide. In a particular embodiment of the present invention, the vaccine formulations of the invention comprises 0.1 mg to 50 mg of CpG oligonucleotide, preferably from 0.2 mg to 10 mg CpG oligonucleotide, more preferably from 0.3 mg to 5 mg CpG oligonucleotide. In a particular embodiment of the present invention, the vaccine formulations of the invention comprises from 0.3 mg to 5 mg CpG oligonucleotide. Even preferably, the vaccine formulations of the invention may comprise from 0.5 to 2 mg CpG oligonucleotide. Most preferably, the vaccine formulations of the invention may comprise from 0.75 to 1.5 mg CpG oligonucleotide. In a preferred embodiment, any of the vaccine formulations disclosed herein may comprise about 1 mg CpG oligonucleotide.

### Liposomal Adjuvants

In one embodiment, the adjuvant comprises liposomes. “Liposomes” as used herein refer to closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose, hydroxyethylcellulose, polyethyleneglycol, polyaspartamide and hydrophilic peptide sequences as described in U.S. Pat. Nos. 6,316,024; 6,126,966; 6,056,973; and 6,043,094. Liposomes can be made without hydrophilic polymers. Therefore, liposome adjuvants may or may not contain hydrophilic polymers. Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phosphatidylglycerol, phosphatidylinositol, and sphingomyelin as disclosed in U.S. Pat. Nos. 6,056,973 and 5,874,104.

A liposomal adjuvant comprises liposomes. When a liposomal adjuvant is used in a vaccine formulation, water-soluble antigens, such as proteins, peptides, nucleic acids, or carbohydrates, are encapsulated in the internal aqueous volume of the liposomes (See Tretiakova et al. Liposomes as Adjuvants and Vaccine Delivery Systems. Biochem (Mosc) Suppl Ser A Membr Cell Biol. 2022; 16 (1): 1-20). Alternatively, when a liposomal adjuvant is combined with lipophilic/amphiphilic substances, such as lipopeptides and glycolipids, these agents are embedded in the lipid bilayer (Id.) Depending on the type of molecule that is combined with the liposomal adjuvant, additional interactions can include associating with the surface of liposomes by adsorption or covalent binding (Id.) Accordingly, in some embodiments, a liposomal adjuvant comprises water-soluble antigens and the antigens are encapsulated in the internal aqueous volume of the liposomes. In some embodiments, water-soluble antigens are proteins, peptides, nucleic acids, or carbohydrates. In some embodiments, a liposomal adjuvant is combined with lipophilic or amphiphilic molecules and these molecules are embedded in the lipid bilayer. In some embodiments, the lipophilic or amphiphilic molecules embedded in the lipid bilayer of the liposome comprise cholesterol, fatty acids, or lipids. In some embodiments, the lipophilic or amphiphilic molecules embedded in the lipid bilayer are lipidated.

Contemplated herein is the use of any liposomal adjuvant. In one embodiment, the liposomal adjuvant is AS01. AS01 comprises 3-O-deacylated monophosphoryl lipid A (3D-MPL) and QS21 in a “quenched form” with cholesterol (See U.S. Pat. No. 10,039,823). In AS01, the lipid bilayer is comprised of a neutral lipid that is “non-crystalline” at room temperature, such as dioleoyl phosphatidylcholine, cholesterol, MPLA, and QS-21 (See U.S. Pat. No. 10,039,823 and WO 1996/033739). During manufacture of AS01, small unilamellar liposomal vesicles (SUV) are first created and purified QS-21 is then added to the SUV. The QS-21 imparts unique properties in that it binds to the liposomal cholesterol where it causes perforations (holes) or other permanent structural changes in the liposomes (See, e.g., Paepenmuller et al., 2014, Int. J. Pharm., 475:138-46). A reduced amount of free QS-21 presumably resulted in reduced local injection pain often caused by free QS-21 (See, e.g., Waite et al., 2001, Vaccine, 19:3957-67; Mbawuike et al., 2007, Vaccine, 25:3263-69). In some embodiments, AS01 contains cholesterol (sterol) at a mole percent concentration of between about 1 and about 50% (mol/mol), preferably between about 20 and about 25% (mol/mol) (See U.S. Pat. No. 10,039,823). In some embodiments, AS01 (including for example, AS01A, AS01B, AS01C, AS01D, AS01E, and AS015) comprises dioleoyl phosphatidylcholine (DOPC), cholesterol, MPLA, for example 3D-MPL, and QS-21. In further embodiments, the liposomal adjuvant is selected from the group consisting of AS01A, AS01B, AS01C, AS01D, AS01E, and AS015. In one embodiment, the liposomal adjuvant is AS01A. In some embodiments, AS01A comprises 3D-MPL, toll-like receptor 4 agonist, and QS-21. In one embodiment, the liposomal adjuvant is AS01B. In some embodiments, AS01B comprises 1000 μg per dose DOPC, 250 μg per dose cholesterol, 50 μg per dose 3D-MPL, 50 μg per dose QS21, phosphate NaCl buffer, and water to a volume of 0.5 ml (See U.S. Pat. No. 10,039,823). In one embodiment, the liposomal adjuvant is AS01E. In some embodiments, AS01E comprises the same components as AS01B but at a lower concentration. In some embodiments, AS01E comprises 500 μg per dose dioleoyl phosphatidylcholine (DOPC), 125 μg per dose cholesterol, 25 μg per dose 3D-MPL, 25 μg per dose QS21, phosphate NaCl buffer, and water to a volume of 0.5 ml (See U.S. Pat. No. 10,039,823). In one embodiment, the liposomal adjuvant is AS015. In some embodiments, AS015 comprises dioleoyl phosphatidylcholine (DOPC), cholesterol, 3D-MPL, QS-21, and CpG.

In one embodiment, the liposomal adjuvant is LiNA-1. In some embodiments, LiNA-1 comprises MPLA and a saponin. In some embodiments, LiNA-1 comprises MPLA and QS-21. In other embodiments, LiNA-1 comprises phosphorylated hexaAcyl disaccharide (PHAD®) (i.e., monophosphoryl lipid A (synthetic) available from Avanti® polar lipids) and QS-21. In another particular embodiment, LiNA-1 comprises PHAD®, QS-21, cholesterol, and DOPC. In another particular embodiment, LiNA-1 comprises 3D-PHAD®, QS-21, cholesterol, and DOPC. In another particular embodiment, LiNA-1 comprises the following components per 0.5 mL dose: (i) 50 μg MPLA (i.e., 3D-PHAD®), (ii) 250 μg cholesterol, (iii) 50 μg QS-21, and (iv) 1000 μg DOPC. In another particular embodiment, LiNA-1 comprises the following components per 0.5 mL dose: (i) 50 μg MPLA (i.e., PHAD®), (ii) 250 μg cholesterol, (iii) 50 μg QS-21, and (iv) 1000 μg DOPC. In some embodiments, the LiNA-1 formulations may be LiNA-1 at 0.0625× concentration (0.0625XLiNA-1), LiNA-1 at 0.125× concentration (0.125XLiNA-1), LiNA-1 at 0.25× concentration (0.25XLiNA-1), LiNA-1 at 0.5× concentration (0.5XLiNA-1), LiNA-1 at 1× concentration (1XLiNA-1), LiNA-1 at 2× concentration (2XLiNA-1), LiNA-1 at 3× concentration (3XLiNA-1), or LiNA-1 at 4× concentration (4XLiNA-1).

In a particular embodiment, the liposomal adjuvant is ALFQ. In some embodiments, ALFQ comprises MPLA and saponin (See U.S. Pat. No. 10,434,167). In some embodiments, ALFQ comprises a lipid bilayer comprising phospholipids in which the hydrocarbon chains have a melting temperature in water of ≥23° C. In further embodiments, ALFQ comprises cholesterol at a mole percent concentration of greater than about 50% (mol/mol). In certain embodiments, ALFQ comprises between about 55% and about 71% (mol/mol) cholesterol. In particular embodiments, ALFQ comprises about 55% (mol/mol) cholesterol. In some embodiments, ALFQ comprises MPLA and QS-21. In other embodiments, ALFQ comprises monophosphoryl 3-deacyl lipid A phosphorylated hexaacyl disaccharide (3D-PHAD®) (i.e., monophosphoryl 3-Deacyl Lipid A (synthetic) available from Avanti® polar lipids) and a saponin. In another particular embodiment, ALFQ comprises 3D-PHAD®, QS-21, dimyristoyl phosphatidylcholine (DMPC), dimyristoyl phosphatidylglycerol (DMPG), and cholesterol. In another particular embodiment, ALFQ comprises (i) 7.0 mg/mL DMPC, (ii) 0.78 mg/ml DMPG, (iii) 5.4 mg/ml cholesterol, (iv) 0.2 mg/mL MPLA (3D-PHAD®), and (v) 0.1 mg/ml QS-21.

In a particular embodiment, the liposomal adjuvant is LiNA-2. In some embodiments, LiNA-2 comprises MPLA and saponin. In some embodiments, LiNA-2 comprises a lipid bilayer comprising phospholipids in which the hydrocarbon chains have a melting temperature in water of ≥23° C. In further embodiments, LiNA-2 comprises cholesterol at a mole percent concentration of greater than about 50% (mol/mol). In certain embodiments, LiNA-2 comprises between about 55% to about 71% (mol/mol) cholesterol. In particular embodiments, LiNA-2 comprises about 55% (mol/mol) cholesterol. In some embodiments, LiNA-2 comprises MPLA and QS-21. In other embodiments, LiNA-2 comprises monophosphoryl 3-deacyl lipid A phosphorylated hexaacyl disaccharide (3D-PHAD®) and a saponin. In another particular embodiment, LiNA-2 comprises 3D-PHAD®, QS-21, dimyristoyl phosphatidylcholine (DMPC), dimyristoyl phosphatidylglycerol (DMPG) and cholesterol.

In some embodiments, the LiNA-2 adjuvant comprises a phosphate buffer. In some embodiments, the LiNA-2 adjuvant comprises a phosphate buffer at a concentration between about 1 mM and about 100 mM. In some embodiments, the LiNA-2 adjuvant comprises a phosphate buffer between about 1 mM and 10 mM. In some embodiments, the LiNA-2 adjuvant comprises a phosphate buffer of about 5 mM, about 10 mM, about 15 mM, about 20 mM, about 25 mM, about 30 mM, about 35 mM, about 40 mM, about 45 mM, or about 50 mM. In a particular embodiment, the LiNA-2 adjuvant comprises a phosphate buffer of about 10 mM. In another particular embodiment, the LiNA-2 adjuvant comprises 3D-PHAD®, QS-21, DMPC, DMPG, cholesterol, and a phosphate buffer. In a further particular embodiment, the LiNA-2 adjuvant comprises 3D-PHAD®, QS-21, DMPC, DMPG, cholesterol, and 10 mM phosphate buffer.

In some embodiments, the LiNA-2 adjuvant comprises sodium chloride. In some embodiments, the LiNA-2 adjuvant comprises between about 50 mM and about 500 mM sodium chloride. In other embodiments, the LiNA-2 adjuvant comprises about 25 mM, about 50 mM, about 75 mM, about 100 mM, about 125 mM, about 150 mM, about 175 mM, about 200 mM, about 225 mM, or about 250 mM sodium chloride. In a particular aspect, the LiNA-2 adjuvant comprises about 150 mM sodium chloride. In one embodiment, the LiNA-2 adjuvant comprises 3D-PHAD®, QS-21, DMPC, DMPG, cholesterol, sodium chloride, and a phosphate buffer. In a further particular embodiment, the LiNA-2 adjuvant comprises 3D-PHAD®, QS-21, DMPC, DMPG, cholesterol, 150 mM sodium chloride, and a 10 mM phosphate buffer.

In one embodiment, the adjuvant formulation is 0.5XLiNA-2 (also known as ALFQ), wherein the 0.5XLiNA-2 may be homogeneous or heterogeneous, comprising (i) 7.0 mg/mL DMPC, (ii) 0.78 mg/ml DMPG, (iii) 5.4 mg/ml cholesterol, (iv) 0.2 mg/mL MPLA (3D-PHAD®), and (v) 0.1 mg/ml QS-21. In another embodiment, the adjuvant formulation is 1XLiNA-2, wherein the 1XLiNA-2 may be homogeneous or heterogeneous, comprising (i) 14±7 mg/mL DMPC, (ii) 1.6±0.8 mg/ml DMPG, (iii) 11±6 mg/ml cholesterol, (iv) 0.40±0.20 mg/mL MPLA (3D-PHAD®), and (v) 0.20±0.10 mg/ml QS-21. In a further embodiment, the adjuvant formulation is 2XLiNA-2, wherein the 2XLiNA-2 may be homogeneous or heterogeneous, comprising (i) 28+14 mg/mL DMPC, (ii) 3.2±1.6 mg/ml DMPG, (iii) 22±11 mg/ml cholesterol, (iv) 0.80±0.40 mg/mL MPLA (3D-PHAD®), and (v) 0.40±0.20 mg/ml QS-21. In some embodiments, the LiNA-2 homogeneous or heterogeneous adjuvant formulations may be LiNA-2 at 0.0625× concentration (0.0625XLiNA-2), LiNA-2 at 0.125× concentration (0.125XLiNA-2), LiNA-2 at 0.25× concentration (0.25XLiNA-2), LiNA-2 at 0.5× concentration (0.5XLiNA-2), LiNA-2 at 1× concentration (1XLiNA-2), LiNA-2 at 2× concentration (2XLiNA-2), LiNA-2 at 3× concentration (3XLiNA-2), or LiNA-2 at 4× concentration (4XLiNA-2).

In some embodiments, the liposomal adjuvant is CAF09 (See Korsholm et al. Induction of CD8+ T-cell responses against subunit antigens by the novel cationic liposomal CAF09 adjuvant, Vaccine, Volume 32, Issue 31, 2014, Pages 3927-3935). In some embodiments, the liposomal adjuvant CAF09 comprises dimethyldioctadecylammonium (DDA), monomycoloyl glycerol (MMG)-1, and polyinosinic-polycytidylic acid (poly I:C).

Phosphatidylcholine phospholipid (PC)/Phosphatidylglycerol phospholipid (PG): In one embodiment wherein the adjuvant comprises liposomes, the liposomes comprise phosphatidylcholine phospholipid (PC). In some embodiments, the PC is selected from the group consisting of: dimyristoyl phosphatidylcholine (DMPC), dipalmitoyl phosphatidylcholine (DPPC), and distearyl phosphatidylcholine (DSPC). In one embodiment wherein the adjuvant comprises liposomes, the liposomes comprise phosphatidylglycerol phospholipid (PG). In some embodiments, the PG is selected from the group consisting of: dimyristoyl phosphatidylglycerol (DMPG), dipalmitoyl phosphatidylglycerol (DPPG), and distearyl phosphatidylglycerol (DSPG). In a further embodiment, the adjuvant comprises a combination of (i) a phosphatidylcholine phospholipid (PC) selected from the group consisting of: dimyristoyl phosphatidylcholine (DMPC), dipalmitoyl phosphatidylcholine (DPPC), and distearyl phosphatidylcholine (DSPC), and (ii) a phosphatidylglycerol phospholipid (PG) selected from the group consisting of: dimyristoyl phosphatidylglycerol (DMPG), dipalmitoyl phosphatidylglycerol (DPPG), and distearyl phosphatidylglycerol (DSPG). In some embodiments, the liposome composition of the adjuvant has a ratio of PC to PG (mol/mol) of about 0.5:1, about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. In a particular embodiment, the liposome composition of the adjuvant comprises PC and PG, wherein the PC is dimyristoyl phosphatidylcholine (DMPC) and the PG is dimyristoyl phosphatidylglycerol (DMPG), having a mole ratio of PC to PG (mol/mol) of about 9:1.

Cholesterol: In some embodiments wherein the adjuvant comprises liposomes, the liposomes of the adjuvant comprise cholesterol. In one embodiment, the liposome composition of the adjuvant formulation comprises cholesterol at a mole percent concentration of over 50% (mol/mol), for example about 55% to about 71% (mol/mol). In a particular embodiment, the adjuvant comprises liposomes that comprise about 55% (mol/mol) cholesterol.

Cholesterol and Phospholipids: In some embodiments wherein the adjuvant comprises liposomes, the liposomes of the adjuvant comprise cholesterol and phospholipids. In some embodiments, the mole ratio of the cholesterol (b) to the phospholipids (a) is about 55:45 to about 71:29. In one embodiment, the mole ratio of the cholesterol (b) to the phospholipids (a) is about 55:50, about 55:45, about 55:40, about 55:35, or about 55:30. In a particular embodiment, the mole ratio of the cholesterol (b) to the phospholipids (a) is about 55:45.

Vesicle Species: In some embodiments wherein the adjuvant comprises liposomes, the liposomes comprise multi-lamellar vesicles (MLV) or small uni-lamellar vesicles (SUV), wherein small uni-lamellar vesicles are about 50 to about 100 nm in diameter, and wherein multi-lamellar vesicles are about 1 to about 4 μm in diameter.

MPLA: In another embodiment wherein the adjuvant comprises liposomes, the liposome composition comprises Lipid A. In another embodiment wherein the adjuvant comprises liposomes, the liposome composition comprises monophosphoryl lipid A (MPLA). In one embodiment, the liposome composition comprises pentaacylated MPLA (P-MPLA). In another embodiment, the liposome composition comprises monophosphoryl lipid A phosphorylated hexaAcyl disaccharide (PHAD®). In a particular embodiment, the MPLA is monophosphoryl 3-deacyl lipid A phosphorylated hexaacyl disaccharide (3D-PHAD®). In one embodiment, the liposome composition comprises about 5 mg or less, about 4 mg or less, about 3 mg or less, about 2 mg or less, about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, or about 0.01 mg or less of MPLA, PHAD®, or 3D-PHAD®, etc. (total weight per ml liposome suspension).

MPLA and Phospholipids: In one embodiment, wherein the adjuvant comprises liposomes, the liposomes comprise MPLA and phospholipids. In another embodiment, wherein the adjuvant comprises liposomes, the liposomes comprise PHAD® or 3D-PHAD® and phospholipids. In one embodiment, the liposome composition of the adjuvant has a MPLA: phospholipid mole ratio of about 1:5.6 to about 1:880, or about 1:88 to about 1:220. In one embodiment, the liposome composition of the adjuvant comprises a PC and a PG, wherein the PC is dimyristoyl phosphatidylcholine (DMPC) and the PG is dimyristoyl phosphatidylglycerol (DMPG), having a MPLA: phospholipid mole ratio of about 1:220, about 1:88 or about 1:5.6, in particular 1:88. In one embodiment, the liposome composition of the adjuvant comprises DMPC, DMPG, and 3D-PHAD® and has a 3D-PHAD®: phospholipid mole ratio between about 1:5 and about 1:6, for example 1:5.6. In one embodiment, the liposome composition of the adjuvant comprises DMPC, DMPG, and 3D-PHAD® and has a 3D-PHAD®.phospholipid mole ratio between about 1:200 and about 1:240, for example 1:220. In another embodiment, the liposome composition of the adjuvant comprises DMPC, DMPG, and 3D-PHAD® and has a 3D-PHAD®: phospholipid mole ratio between about 1:80 and about 1:95. In another particular embodiment, the liposome composition of the adjuvant formulation comprises DMPC, DMPG, and 3D-PHAD® and has a 3D-PHAD®: phospholipid mole ratio of about 1:88.

Saponin: In another embodiment, the adjuvant comprises liposomes that comprise a saponin. In some embodiments, the saponin is Quil A, its derivatives thereof, or any purified component thereof (for example, QS-7, QS-18, QS-21, or a mixture thereof). In a particular embodiment, the adjuvant comprises liposomes which comprise QS-21. In some embodiments, the adjuvant formulation has a content of saponin (total weight per ml liposome suspension) of about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, or about 0.01 mg or less. In a particular embodiment, the adjuvant formulation comprises a content of saponin of about 0.15 to 0.4 mg/ml.

MPLA and Saponin: In another embodiment wherein the adjuvant comprises liposomes, the adjuvant comprises a MPLA-containing liposome composition and at least one saponin (e.g., QS-21). In another embodiment, the adjuvant comprises a monophosphoryl lipid A (MPLA)-containing liposome composition and at least one saponin, wherein the liposome composition comprises i) a lipid bilayer comprising phospholipids and ii) cholesterol at a mole percent concentration of the liposome composition of greater than about 50% (mol/mol). The saponin may be QS-7, QS-18, QS-21, or a mixture thereof. In a particular embodiment, the saponin is QS-21. In another embodiment, the adjuvant comprises a MPLA-containing liposome that comprises (1) a lipid bilayer comprising phospholipids in which the hydrocarbon chains have a melting temperature in water of ≥23° C., usually dimyristoyl phosphatidylcholine (DMPC, e.g. 1,2-dimyristoyl-sn-glycero-3-phosphocholine) and dimyristoyl phosphatidylglycerol (DMPG, e.g. 1,2-dimyristoyl-sn-glycero-3-phospho-(1′-rac-glycerol)); (2) cholesterol (Chol) as a stabilizer; and (3) monophosphoryl lipid A (MPLA) as an immunostimulator.

Homogenous Liposomes: In another embodiment, the adjuvant comprises homogenous liposomes. In one embodiment, the adjuvant comprises homogenous liposomes that range in size from between about 1 nm and about 500 nm. In some embodiments, the homogenous liposomes within the adjuvant range in size from between about 10 nm, about 20 nm, about 30 nm, about 40 nm, or about 50 nm and about 400 nm. In other embodiments, the homogenous liposomes within the adjuvant range in size from between about 10 nm, about 20 nm, about 30 nm, about 40 nm, or about 50 nm and about 300 nm. In other embodiments, the homogenous liposomes within the adjuvant range in size from between about 10 nm, about 20 nm, about 30 nm, about 40 nm, or about 50 nm and about 200 nm. In some embodiments, the homogenous liposomes within the adjuvant have a size of less than about 300 nm, about 250 nm, about 200 nm, about 150 nm, or about 100 nm. In a particular embodiment, the homogenous liposomes within the adjuvant have a size of less than about 200 nm. In one embodiment, the homogeneous liposomes have a polydispersity index (PDI) between about 0.05, about 0.1, about 0.015, or about 0.2 and about 0.3, about 0.35, about 0.4, about 0.45, or about 0.5. In some embodiments, the homogenous liposomes that have a PDI less than about 0.3, about 0.35, about 0.4, about 0.45, or about 0.5. In a particular embodiment, the homogenous liposomes within the adjuvant have a PDI of less than about 0.3.

Heterogenous Liposomes: In another embodiment, the adjuvant comprises heterogenous liposomes. In one embodiment, the heterogeneous liposomes range in size from between about 1 nm and about 10 μM. In some embodiments, the heterogenous liposomes range in size from between about 30 nm and about 4 μM. In other embodiments, the heterogenous liposomes range in size from between about 30 nm and about 1400 nm. In still other embodiments, the heterogenous liposomes range in size from between about 30 nm and about 1000 nm. In some embodiments, the heterogenous liposomes range in size from between about 100 nm, about 200 nm, about 300 nm, about 400 nm, or about 500 nm and about 1000 nm. In a particular embodiment, the heterogenous liposomes within the adjuvant range in size from between about 300 nm and about 1000 nm. In other embodiments, the heterogenous liposomes within the adjuvant have a size of greater than about 500 nm, about 400 nm, about 300 nm, about 200 nm, or about 100 nm. In a particular embodiment, the heterogenous liposomes within the adjuvant have a size of greater than 300 nm. In another embodiment, the heterogeneous liposomes have a polydispersity index (PDI) between about 0.4 and about 1. In some embodiments, the heterogeneous liposomes have a PDI about 0.4, about 0.5, about 0.6, about 0.7, about 0.8, about 0.9, about 1, or more. In a particular embodiment, the heterogeneous liposomes of the adjuvant have a PDI of more than about 0.4. In another particular embodiment, the heterogeneous liposomes of the adjuvant have a PDI of more than about 0.5.

In one embodiment wherein the adjuvant comprises liposomes, the adjuvant is ALFQ comprising homogenous liposomes. In another embodiment wherein the adjuvant comprises liposomes, the adjuvant is ALFQ comprising heterogenous liposomes. In another particular embodiment wherein the adjuvant comprises liposomes, the adjuvant is LiNA-2 comprising homogenous liposomes (as referred to as LiNA-2A). In another particular embodiment wherein the adjuvant comprises liposomes, the adjuvant is LiNA-2 comprising heterogenous liposomes (as referred to as LiNA-2B).

In an embodiment, the formulation includes 1, 2, 3, or more adjuvants. In one embodiment, the formulation comprises one adjuvant, wherein the adjuvant comprises aluminum phosphate. In another embodiment, the formulation comprises two adjuvants, one of which comprises aluminum phosphate. In one embodiment, the formulation comprises one adjuvant, wherein the adjuvant comprises liposomes. In another embodiment, the formulation comprises at least two adjuvants, one of which comprises liposomes. In one embodiment, the formulation comprises one adjuvant, wherein the adjuvant comprises MPLA and a saponin. In another embodiment, the formulation comprises at least two adjuvants, one of which comprises MPLA and a saponin. In one embodiment, the formulation comprises one adjuvant, wherein the adjuvant comprises LiNA-2. In another embodiment, the formulation comprises at least two adjuvants, one of which is LiNA-2. In another particular embodiment, the formulation comprises aluminum phosphate and LiNA-2 as adjuvants. In another particular embodiment, the formulation comprises only an aluminum phosphate adjuvant. In another particular embodiment, the formulation comprises only a LiNA-2 adjuvant.

Also provided is an immunogenic composition comprising an immunogen and an adjuvant, described herein. The immunogenic composition may typically comprise a physiologically acceptable vehicle. The immunogen of the immunogenic composition can be selected from the group consisting of a naturally-occurring or artificially-created protein, a recombinant protein, a glycoprotein, a peptide, a carbohydrate, a hapten, a whole virus, a bacterium, a protozoan, and a virus-like particle. A method of immunizing an animal comprising administering the immunogenic composition is also provided.

In particular embodiments, the immunogenic composition comprises components in suspension that sediment over time and the adjuvant described herein is for use in the resuspension of the components of the immunogenic composition. In some embodiments, the immunogenic composition is stored in a container. In particular embodiments, the immunogenic composition is stored in syringe, for example a pre-filled syringe (PFS). In other particular embodiments, the immunogenic composition comprises aluminum. In some embodiments, the aluminum is aluminum phosphate. In still other particular embodiments, the adjuvant comprises liposomes. In some embodiments, the adjuvant comprises MPLA and a saponin. In particular embodiments, the adjuvant is LiNA-2. In particular embodiments, the adjuvant is 1XLiNA-2, either homogenous or heterogenous. In still other particular embodiments, the adjuvant is 2X-LiNA-2, either homogenous or heterogenous.

In some embodiments, the container storing the immunogenic composition is stored at a temperature between about 1° C. and about 20° C. In some embodiments, the container storing the immunogenic composition is stored at a temperature of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25° C. In some embodiments, the container storing the immunogenic composition is stored at a temperature between about 2° C. and about 8° C.

In some embodiments, at time T0 substantially all the components in the stored immunogenic composition are in suspension. In other embodiments, at time T0 substantially all the components in the stored immunogenic composition are fully dispersed. In still other embodiments, at time T0 the immunogenic composition is substantially fully homogenous.

In some embodiments, at time T1 between about 1% and about 100% of the components in the immunogenic composition have sedimented out of suspension. In other embodiments, at time T1 between about 25% and about 100% of the components in the immunogenic composition have sedimented out of suspension. In still other embodiments, at time T1 between about 50% and about 100% of the components in the immunogenic composition have sedimented out of suspension. In some embodiments, at time T1 between about 75% and about 100% of the components in the immunogenic composition have sedimented out of suspension. In other embodiments, at time T1 between about 90% and about 100% of the components in the immunogenic composition have sedimented out of suspension. In still other embodiments, at time T1 between about 95% and about 100% of the components in the immunogenic composition have sedimented out of suspension. In some embodiments at time T1, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the components in the immunogenic composition have sedimented out of suspension. In some embodiments, T1 is between about 1 days and about 100 days, or more. In some embodiments, T1 is between at about 1 days and about 30 days, or more. In still other embodiments, T1 is between about 25 days and about 35 days, or more. In a particular embodiment, T1 is about 30 days. In additional embodiments, T1 is between about 5 days and about 10 days, or more. In a particular embodiment, T1 is about 7 days. In still other embodiments, T1 is between about 1 day and about 5 days, or more. In a particular embodiment, T1 is about 2 days.

In a particular embodiment, the adjuvant described herein reduces the number of handshakes required to resuspend the immunogenic composition at T1, compared to the immunogenic composition without the adjuvant. In some embodiments, the adjuvant reduces the number of handshakes required to resuspend the immunogenic composition at T1 by between about 1 and about 200 handshakes, or more. In additional embodiments, the adjuvant reduces the number of handshakes required to resuspend the immunogenic composition at T1 by between about 1 and about 100 handshakes, or more. In some embodiments, the adjuvant reduces the number of handshakes required to resuspend the immunogenic composition at T1 by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 handshakes. In one embodiment, the adjuvant reduces the number of handshakes required to resuspend the composition at T1 by between about 5 and about 10 handshakes, or more. In a particular embodiment, the adjuvant reduces the number of handshakes required to resuspend the composition at T1 by about 6 or about 8 handshakes. In one embodiment, the adjuvant reduces the number of handshakes required to resuspend the composition at T1 by between about 15 and about 20 handshakes. In a particular embodiment, the adjuvant reduces the number of handshakes required to resuspend the composition at T1 by about 18 or about 19 handshakes. In one embodiment, the adjuvant reduces the number of handshakes required to resuspend the composition at T1 by between about 20 and about 30 handshakes, or more. In a particular embodiment, the adjuvant reduces the number of handshakes required to resuspend the composition at T1 by about 22 handshakes.

In other particular embodiments, the adjuvant reduces the number of handshakes required to resuspend the composition at T1 by about 29, about 28, or about 27 handshakes. In still another embodiment, the adjuvant reduces the number of handshakes required to resuspend the composition at T1 by between about 35 and about 45 handshakes, or more. In a particular embodiment, the adjuvant reduces the number of handshakes required to resuspend the composition at T1 by about 41 handshakes. In yet another embodiment, the adjuvant reduces the number of handshakes required to resuspend the composition at T1 by between about 60 and about 70 handshakes, or more. In a particular embodiment, the adjuvant reduces the number of handshakes required to resuspend the composition at T1 by about 64 handshakes.

In some embodiments, the resuspended immunogenic composition is homogenous. In additional embodiments, the resuspended immunogenic composition is fully dispersed. In further embodiments, the resuspended immunogenic composition appears uniform in color.

### Uses of the Vaccine Formulations of the Invention

In an embodiment, the vaccine formulations disclosed herein are for use as a medicament.

The vaccine formulations described herein may be used in various therapeutic or prophylactic methods for preventing, treating or ameliorating a bacterial infection, disease or condition in a subject. In particular, vaccine formulations described herein may be used to prevent, treat or ameliorate a S. pneumoniae infection, disease or condition in a subject.

In one aspect, the invention provides a method of preventing, treating or ameliorating an infection, disease or condition in a subject associated with one or more of the following: chickenpox or shingles, human respiratory syncytial virus infection (RSV), Cytomegalovirus infection (CMV), Human metapneumovirus, Human parainfluenza viruses type 1 or type 3, Lyme disease, Streptococcus pneumonia, Clostridioides difficile, Coronaviruses, Escherichia coli, Klebsiella pneumoniae, influenza, HIV-1, Hepatitis A, Hepatitis B, Human Papilloma virus, Meningococcal type A meningitis, Meningococcal type B meningitis, Meningococcal type C meningitis, Meningococcal type W meningitis, Meningococcal type Y meningitis, Tetanus, Diphtheria, Pertussis, Polio, Haemophilus influenza type B, Dengue, Hand Foot and Mouth Disease, Typhoid, Pneumococcus, Japanese encephalitis virus, Anthrax, Shingles, Malaria, Norovirus, and cancer, comprising administering to the subject an immunologically effective amount of a vaccine formulation of the invention.

In a particular aspect, the invention provides a method of preventing, treating or ameliorating an infection, disease or condition associated with S. pneumoniae in a subject, comprising administering to the subject an immunologically effective amount of a vaccine formulation of the invention.

In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In one embodiment, the invention provides a method of inducing an immune response in a subject to one or more of the following: chickenpox or shingles, human respiratory syncytial virus infection (RSV), Cytomegalovirus infection (CMV), Human metapneumovirus, Human parainfluenza viruses type 1 or type 3, Lyme disease, Streptococcus pneumonia, Clostridioides difficile, Coronaviruses, Escherichia coli, Klebsiella pneumoniae, influenza, HIV-1, Hepatitis A, Hepatitis B, Human Papilloma virus, Meningococcal type A meningitis, Meningococcal type B meningitis, Meningococcal type C meningitis, Meningococcal type W meningitis, Meningococcal type Y meningitis, Tetanus, Diphtheria, Pertussis, Polio, Haemophilus influenza type B, Dengue, Hand Foot and Mouth Disease, Typhoid, Pneumococcus, Japanese encephalitis virus, Anthrax, Shingles, Malaria, Norovirus, and cancer, comprising administering to the subject an immunologically effective amount of a vaccine formulation described herein.

In an embodiment, the invention provides a method of inducing an immune response to S. pneumoniae in a subject comprising administering to the subject an immunologically effective amount of a vaccine formulation of the invention.

In an embodiment, the vaccine formulations disclosed herein are for use as a vaccine. In some embodiments, the vaccine formulations described herein may be used to prevent a bacterial or viral infection in a subject. In such embodiments the vaccine formulations described herein may be used to prevent a S. pneumoniae infection in a subject. Thus, in one aspect, the invention provides a method of preventing a bacterial or viral infection in a subject comprising administering to the subject an immunologically effective amount of a vaccine formulations of the invention. Thus, in one aspect, the invention provides a method of preventing an infection by S. pneumoniae in a subject comprising administering to the subject an immunologically effective amount of a vaccine formulations of the invention. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject to be vaccinated is a mammal, such as a human, cat, sheep, pig, horse, bovine or dog.

In one aspect, the vaccine formulations disclosed herein are for use in a method of preventing, treating or ameliorating a bacterial or viral infection, disease or condition in a subject. In one aspect, the vaccine formulations disclosed herein are for use in a method of preventing, treating or ameliorating an infection, disease or condition associated with S. pneumoniae in a subject. In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In an embodiment, the vaccine formulations disclosed herein are for use as a vaccine. In such embodiments the vaccine formulations described herein may be used to prevent a S. pneumoniae infection in a subject. Thus, in one aspect, the vaccine formulations disclosed herein are for use in a method of preventing, an infection by S. pneumoniae in a subject. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject to be vaccinated is a mammal, such as a human, cat, sheep, pig, horse, bovine or dog.

The vaccine formulations of the present invention can be used to protect or treat a human susceptible to bacterial or viral infection, by means of administering the formulations via a systemic or mucosal route. The vaccine formulations of the present invention can be used to protect or treat a human susceptible to pneumococcal infection, by means of administering the formulations via a systemic or mucosal route. In an embodiment, the vaccine formulations disclosed herein are administered by intramuscular, intraperitoneal, intradermal or subcutaneous routes. In an embodiment, the vaccine formulations disclosed herein are administered by intramuscular, intraperitoneal, intradermal or subcutaneous injection. In an embodiment, the vaccine formulations disclosed herein are administered by intramuscular or subcutaneous injection.

Presented below are examples discussing the vaccine formulations contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Preparation of Vaccine Formulations

It is known that with an increase in serotypes and/or serotype concentration in an adjuvant containing vaccine formulation, resuspension may become more difficult, especially in a pre-filed syringe. It has been observed that the settling behavior of aluminum phosphate containing formulations correlates with resuspension properties when it is placed in a container. When particles in a solution exhibit attractive or low repulsive forces they tend to aggregate as floccules and settle fast. Upon settling it forms a loose pellet with a higher bed height that is easier to resuspend. On the contrary, when particles in a solution exhibit strong repulsive forces they likely settle at a slower rate. Once settled they form a tight cake with a lower bed height that is harder to resuspend. Resuspension may be made easier by modulating the formulation to alter aluminum surface charge by adding different electrolyte excipients or changing formulation conditions. The formulations of the present invention were designed to precipitate at a faster rate so that the cake is not tight and resuspension of the formulation is easier.

The formulated bulk vaccine samples were prepared by adding the required volume of stock solutions of buffers, NaCl, CaCl₂) or sodium phosphate and polysorbate 80, into a sterile formulation container to achieve the desired concentrations of each matrix described below in Table 1. The pneumococcal conjugates were then added individually to container. The preparation was mixed to homogeneity, aluminum phosphate was added, followed by further mixing to allow binding of pneumococcal conjugates.

Different vaccine formulations were analyzed for settling velocity, cake height after complete sedimentation, resuspension in pre-filled syringes and aluminum binding. The formulations and control are described in Table 1. The 20v control (20 serotype control) comprises capsular polysaccharide antigens derived from S. pneumoniae serotypes 1, 3, 4, 5, 6A, 6B, 7F, 8, 9V, 10A, 11A, 12F, 14, 15B, 18C, 19A, 19F, 22F, 23F and 33F with each saccharide individually conjugated to diphtheria cross reactive material (CRM₁₉₇). The 25v control (25 serotype control) comprises glycoproteins derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B with each saccharide individually conjugated to diphtheria cross reactive material (CRM₁₉₇).

TABLE 1

| Formulation # | Formulation Matrix |
|---|---|
| 1 | 20vPnC in 5 mM Succinate pH 5.8, 150 mM NaCl, |
| 20 serotype | 0.02% PS80, 0.25 mg/mL aluminum as AlPO₄ |
| vaccine control | |
| 2 | 25vPnC in 5 mM Succinate pH 5.8, 150 mM NaCl, |
| | 0.02% PS80, 0.25 mg/mL aluminum as AlPO₄ |
| 3 | 25vPnC in 20 mM Sodium Phosphate pH 7.0, 245 mM |
| | NaCl, 0.02% PS80, 0.25 mg/mL aluminum as AlPO₄ (<2 |
| | mM succinate carryover from DS) |
| 4 | 25vPnC in 5 mM Succinate pH 5.8, 150 mM NaCl, 20 |
| | mM CaCl₂, 0.02% PS80, 0.25 mg/mL aluminum as |
| | AlPO₄ |
| 5 | 25vPnC in 5 mM Succinate pH 5.8, 150 mM NaCl, 40 |
| | mM MgCl₂, 0.02% PS80, 0.25 mg/mL aluminum as |
| | AlPO₄ |
| 6 | 25vPnC in 5 mM Succinate pH 5.8, 40 mM Sodium |
| | Phosphate, 245 mM NaCl, 0.02% PS80, 0.25 mg/mL |
| | aluminum as AlPO₄ |
| 7 | 25vPnC in 30 mM Histidine pH 5.8, 245 mM NaCl, |
| | 0.02% PS80, 0.25 mg/mL aluminum as AlPO₄ (<2 mM |
| | succinate carryover from DS) |
| 8 | 25vPnC in 30 mM Histidine pH 5.8 20 mM Sodium |
| | Phosphate, 245 mM NaCl, 0.02% PS80, 0.25 mg/mL |
| | aluminum as AlPO₄ (<2 mM succinate carryover from |
| | DS) |
| 9 | 25vPnC in 5 mM Succinate pH 5.8, 150 mM NaCl, |
| | 0.02% PS80, 0.50 mg/mL aluminum as AlPO₄ |
| 10 | 25vPnC in 30 mM Histidine pH 5.8, 245 mM NaCl, |
| | 0.02% PS80, 0.50 mg/mL aluminum as AlPO₄ |

*20vPnC and 25vPnC formulation contains 4.4 μg/mL of each serotype except 8.8 μg/mL for 6B (see text above for list of all conjugates)

### Example 2

### Sedimentation Analyses

It is known from past manufacturing experience that a faster sedimentation rate can increase the risk of challenges with sample homogeneity during the filling process. The goal was to select an optimal formulation for a 25 serotypes pneumococcal vaccine that settles no faster than a seven serotypes control vaccine to circumvent potential manufacturing challenges, and no slower than a 20 serotypes pneumococcal vaccine in order to improve resuspension. Two formulations were analyzed as controls 20 serotypes vaccine control (Formulation #1-20 serotypes in 5 mM Succinate pH 5.8, 150 mM NaCl, 0.02% PS80, 0.5 mg/mL AlPO₄); Control B (7 serotypes in 5 mM Succinate pH 5.8, 150 mM NaCl, 0.02% PS80, 0.5 mg/mL AlPO₄. The seven serotype vaccine control comprises glycoproteins derived from S. pneumoniae serotypes 4, 6B, 9V, 14, 18C, 19F and 23F formulation in 5 mM Succinate pH 5.8, 150 mM NaCl, 0.02% PS80, 0.25 mg/mL AlPO₄.

The sample settling velocity was assessed using Turbiscan®. A Turbiscan® TOWER (Formulaction, Toulouse, France) was used to monitor the sedimentation properties of aluminum phosphate suspensions under gravitational force. Turbiscan® TOWER uses static multiple light scattering to detect particle migration in liquid dispersions. A measurement head is outfitted with a pulsed near-infrared light source (2=880 nm), and synchronous transmission (180° from light source) and backscattering (45° from light source) detectors which move along the height of a flat-bottomed cylindrical glass sample cell, collecting data every 20 μm. Measurements were performed at room temperature using ~20 mL of sample. Samples were mixed immediately before the measurement. The settling onset time is defined as the time where the sample reaches 45% clarification at the meniscus and was obtained from the transmission data. Sedimentation rate was reported as the slope of change in the sedimentation front position as a function of time (at the midpoint of sedimentation).

FIG. 1 shows the sedimentation of different formulations, from left to right represents fast to slow settling. There are two formulations in Table 1 with sedimentation rates that fell between the serotype control vaccines including formulation #4 (containing 20 mM CaCl₂)), formulation #6 (containing succinate+40 mM sodium phosphate) Formulation #5 (MgCl₂) and formulation #7 (containing 25 mM histidine) exhibited similar or slightly faster sedimentation rates than the 20 serotype vaccine control (Formulation #1). The other formulations are either faster settling than the seven serotypes vaccine control or slower settling than the 20 serotype vaccine control

### Example 3

### Cake Height Analysis

The settling behavior was observed visually in 10 mL graduated cylinders each filled with 10 mL of each formula. Upon complete sedimentation after 2 weeks at room temperature, cake height was measured in millimeters (mm), which was then normalized by mg of aluminum (from AlPO₄ concentration in mg/mL) and by surface area (80 mm² for 10 mL graduated cylinders).

$$Normalized Cake Height ( 1 / mg * mm ) = \frac{Measured Cake Height ( mm )}{mg {AlPO}_{4} * surface area ( {mm}^{2} )}$$

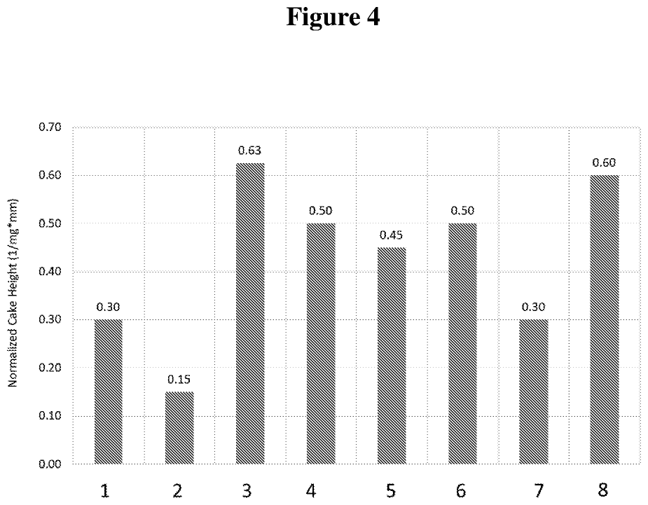

The cake height results are shown in FIG. 4 aligned well with the Turbiscan data and confirmed the previous conclusions. The cake heights of the CaCl₂) formulation (#4 in Table 1) and the Suc+Phos formulation (#3 in Table 1) were both 0.5, which was between the seven serotypes vaccine control (0.65) and the 20 serotype vaccine control (0.3, Formulation #1 in Table 1). The histidine formulation (#7 in Table 1) which had a comparable sedimentation rate to the 20 serotype vaccine control in Turbiscan® had the same cake height of 0.3 as the 20 serotype vaccine control.

### Example 4

### Resuspension

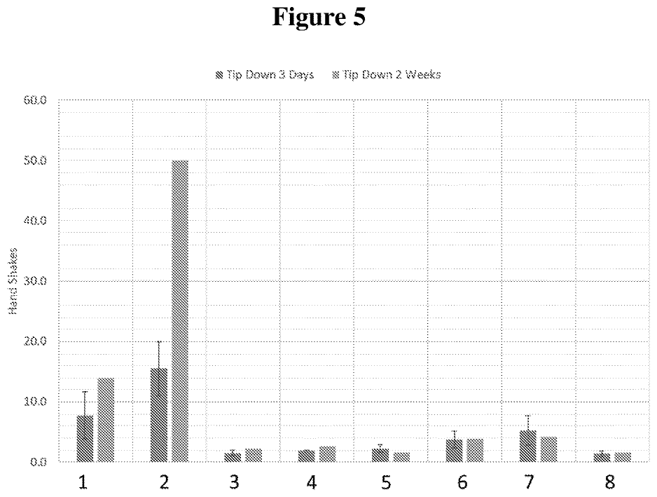

Resuspension in a pre-filled syringe was assessed using the number of handshakes required to achieve a visually homogenous, white suspension. 1 mL glass syringes were filled with each of the formulations from Table 1 to a fill volume of 0.58 mL, stoppered, and stored at 5° C. with tip down orientation. At each timepoint, the filled syringes were removed from the storage tub and allowed to equilibrate to room temperature for ~30 minutes. The syringe was then held horizontally between the forefinger and the thumb of the operator; with the tip pointing away from the operator. The syringe was shaken once by a quick flick of the wrist forwards away from the operator and the syringe is returned to the original starting position at the end of the shake while maintaining the horizontal orientation. Four syringes per orientation were shook, and the average number of handshakes required for achieving a visually homogenous, white suspension was reported. Resuspension was tested at 3-days and again at a 2-week time point. The results showed that all the samples in this study were easier to resuspend than the 20 serotype vaccine control and the 25 serotype vaccine controls (FIG. 5).

### Example 5

### Aluminum Binding

Aluminum binding was evaluated for the samples by Nephelometry. The percent aluminum bound antigenicity for each formulation was tested for the 20 serotypes contained in the 20 serotype vaccine control using serotype specific antibodies. The data in Table 2 shows that the presence of sodium phosphate (Formulation #3, #6, and #8) significantly decreases the aluminum binding across all the serotypes and the presence of CaCl₂) (Formulation #4) slightly increased the binding of some serotypes while other formulation changes had no major impact on the binding compared to the controls.

TABLE 2

| | 1 | 3 | 4 | 5 | 6A | 6B | 7F | 9V | 14 | 18C | 19A | 19F | 23F | 15B | 22F | 33F | 8 | 10A | 11A | 12F |
|---|---|---|---|---|---|---|---|---|---|---|---|---|---|---|---|---|---|---|---|---|
| 1 | 91 | 95 | 28 | 25 | 40 | 99 | 64 | 40 | 41 | 53 | 66 | 88 | 61 | 48 | 52 | 25 | 20 | 53 | 107 | 62 |
| 2 | 87 | 95 | 34 | 27 | 32 | 100 | 57 | 36 | 34 | 47 | 55 | 84 | 59 | 39 | 47 | 30 | 24 | 47 | 107 | 57 |
| 3 | 13 | 0 | 0 | 0 | 10 | 0 | 21 | 16 | 16 | 2 | 7 | 10 | 7 | 19 | 12 | 2 | 3 | 10 | 5 | 4 |
| 4 | 107 | 102 | 30 | 21 | 61 | 99 | 46 | 46 | 30 | 75 | 66 | 84 | 73 | 55 | 52 | 46 | 25 | 71 | 73 | 73 |
| 5 | 100 | 96 | 36 | 25 | 50 | 91 | 34 | 39 | 8 | 64 | 59 | 81 | 66 | 52 | 43 | 41 | 23 | 59 | 107 | 61 |
| 6 | 35 | 6 | 14 | 16 | 20 | 24 | 44 | 38 | 36 | 10 | 13 | 18 | 18 | 23 | 24 | 8 | 14 | 22 | 70 | 53 |
| 7 | 80 | 100 | 17 | 10 | 29 | 106 | 41 | 24 | 21 | 40 | 52 | 86 | 51 | 33 | 31 | 19 | 17 | 39 | 78 | 48 |
| 8 | 23 | 0 | 0 | 0 | 10 | NA | 38 | 24 | 27 | 2 | 2 | 18 | 7 | 17 | 15 | 0 | 8 | 12 | 17 | 36 |

Although the invention has been described with reference to the above examples, it will be understood that modifications and variations are encompassed within the spirit and scope of the invention. Accordingly, the invention is limited only by the following claims.

### Example 6

### PRE-FILLED SYRINGE REDISPERSION WITH AND WITHOUT LINA-1 or LiNA-2

There is an observed increase in resuspension difficulty of pre-filled syringes (PFS) of the 20vPnC formulation after storage at 2-8° C. In this example, the effect of addition of LiNA-2A, LiNA-2B, and LiNA-1 (each described above) on the redispersion of a sample in a pre-filled syringe (PFS) was tested.

### Experiment 1

The samples tested included aluminum phosphate only (AlPO₄), AlPO₄+20-valent pneumococcal conjugate vaccine (20vPnC), AlPO₄+LiNA-2A, and AlPO₄+LiNA-2A+20vPnC. All the samples contained 0.25 mg/ml AlPO₄, 150 mM NaCl, 5 mM succinate, and 0.02% PS80. The samples with 20vPnC contained each of the 20 serotypes at a concentration of 4.4 μg/mL except for serotype 6B was had a concentration of 8.8 μg/mL. The samples that included LiNA-2A contained 3D-PHAD® at 0.4 mg/mL, DMPC at 14 mg/mL, DMPG at 1.6 mg/mL, cholesterol at 11 mg/ml, and QS-21 at 0.2 mg/ml.

To assess the redispersion behavior, each sample was filled into a PFS and stored tip down in a 2-8° C. stability chamber. The PFS were stored and pulled at the following specific timepoints: T2D (2 days), T7D (7 days), and T30D (30 days) after storage. At each time point, the PFS were carefully taken out of the stability chamber and laid horizontally on a bench top to bring the samples to room temperature (approximately 15 minutes). The number of handshakes (as described in Example 4 above) required until a homogenous and fully dispersed suspension was reached were counted for each sample. The number of replicates for each sample was 3.

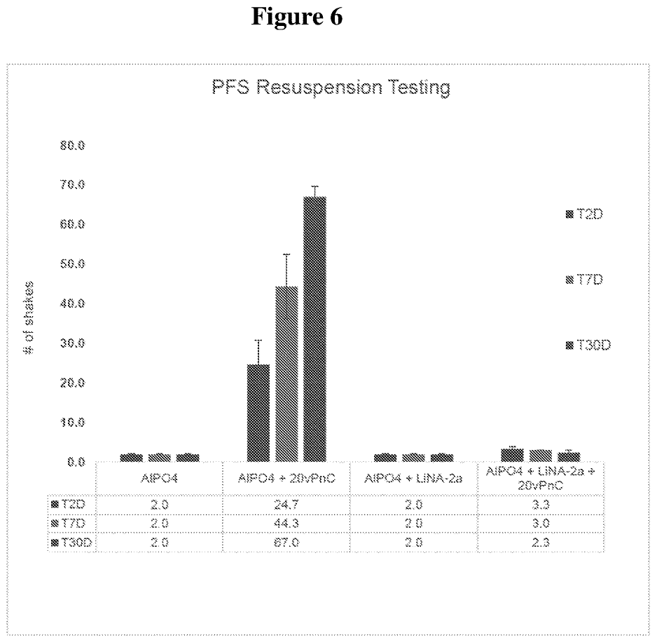

As shown in FIG. 6, the AlPO₄ sample took an average of 2 handshakes to fully redisperse the sample for all pull timepoints tested. Aging of the PFS did not increase the resuspension difficulty of the AlPO₄ sample.

For the AlPO₄+20vPnC sample, the number of handshakes required were an average of 24.6 for T2D, 44.3 for T7D time point, and 67.0 for T30D. As the AlPO₄ and 20vPnC PFS aged, the number of handshakes needed to redisperse the samples increased.

For the AlPO₄+LiNA-2A sample, an average of 2 handshakes to fully redisperse the sample were required for all time points, thus there was no aging effect on this sample.

For the AlPO₄+LiNA-2A+20vPnC sample, an average of about 3 handshakes of the PFS were required to fully redisperse the sample for all timepoints, thus there was no aging effect on this sample.

It was concluded that the presence of LiNA-2A substantially decreased the number of handshakes required to redisperse the PFS sample containing AlPO₄ 20vPnC. Thus, this experiment shows that LiNA-2A is effective in facilitating the resuspension of the conjugate vaccine.

### Experiment 2

The samples tested included AlPO₄+20vPnC, AlPO₄+0.0625XLiNA-2B+20vPnC, AlPO₄+0.125XLiNA-2B+20vPnC, AlPO₄+0.25XLiNA-2B+20vPnC, 0.25×AlPO₄+0.25XLiNA-2B+20vPnC, and 0.25XLiNA-2B+20vPnC. All the AlPO₄ containing samples included 0.25 mg/ml AlPO₄. All the samples further included 150 mM NaCl, 5 mM succinate, and 0.02% PS80. The samples with 20vPnC contained each of the 20 serotypes at a concentration of 4.4 μg/mL except for serotype 6B was had a concentration of 8.8 μg/mL. The samples that included 0.25XLiNA-2B contained 3D-PHAD® at 0.1 mg/mL, DMPC at 3.5 mg/mL, DMPG at 0.4 mg/mL, cholesterol at 2.75 mg/ml, and QS-21 at 0.05 mg/ml. The samples that included 0.125XLiNA-2B contained 3D-PHAD® at 0.05 mg/mL, DMPC at 1.75 mg/mL, DMPG at 0.2 mg/mL, cholesterol at 1.375 mg/ml, and QS-21 at 0.025 mg/ml. The samples that included 0.0625XLiNA-2B contained 3D-PHAD® at 0.025 mg/mL, DMPC at 0.875 mg/mL, DMPG at 0.1 mg/mL, cholesterol at 0.687 mg/ml, and QS-21 at 0.0125 mg/ml.

To assess the redispersion behavior, the PFS storage experiment described in Experiment 1 was repeated, except that PFS were assessed at the following timepoints after storage: TOD (0 days), T7D (7 days), and T30D (30 days).

As shown in Table 3 below, the sample with AlPO₄+20vPnC took the most handshakes to resuspend with 10 at T0, 9 at T7D, and 30 at T30D.

The addition of LiNA-2B significantly reduced the number of handshakes required to resuspend the AlPO₄+20vPnC. For example, in the case of the 0.25× LiNA-2B+AlPO₄+20vPnC, only two handshakes were required to resuspend the sample at T30D.

TABLE 3
Resuspension Results LiNA-2B

| Sample | T0 | T7 D | T30 D |
|---|---|---|---|
| AlPO4 + 20vPnC | 10 | 9 | 30 |
| AlPO4 + 20vPnC + 0.25x LiNA-2B | 1 | 1 | 2 |
| AlPO4 + 20vPnC + 0.125x LiNA-2B | 2 | 1 | 1 |
| AlPO4 + 20vPnC + 0.0625x LiNA-2B | 3 | 3 | 3 |
| 0.25x AlPO4 + 20vPnC + 0.25x LiNA-2B | 1 | 1 | 2 |
| 20vPnC + 0.25x LiNA-2B | 1 | 1 | 3 |

It was concluded that the presence of LiNA-2B substantially decreased the number of handshakes required to redisperse the PFS sample containing AlPO₄ and 20vPnC. Thus, this experiment shows that LiNA-2B is effective in facilitating the resuspension of the conjugate vaccine.

### Experiment 3

The samples tested included AlPO4, AlPO4+20vPnC, AlPO4+LiNA-1, AlPO4+LiNA-1+20vPnC, LiNA-1+20vPnC, and AlPO₄+LiNA-1 buffer only +20vPnC. All the AlPO₄ containing samples included 0.25 mg/ml AlPO₄. All the samples further included 150 mM NaCl, 5 mM succinate, and 0.02% PS80. The samples with 20vPnC contained each of the 20 serotypes at a concentration of 4.4 μg/mL except for serotype 6B was had a concentration of 8.8 μg/mL. The samples that contained LiNA-1 or LiNA-1 buffer only further contained 0.3 mg/mL sodium phosphate dibasic heptahydrate and 0.54 mg/mL monobasic potassium phosphate. The samples that contained LiNA-1 further contained 0.1 mg/mL 3D-PHAD®, 2.0 mg/mL DOPC, 0.5 mg/mL cholesterol, and 0.1 mg/mL QS-21.

To assess the redispersion behavior, the PFS storage experiment described in Experiment 1 was repeated, except that PFS were assessed at the following timepoints after storage: TOD (0 days), T7D (7 days), and T30D (30 days). The number of replicates for each sample was 3.

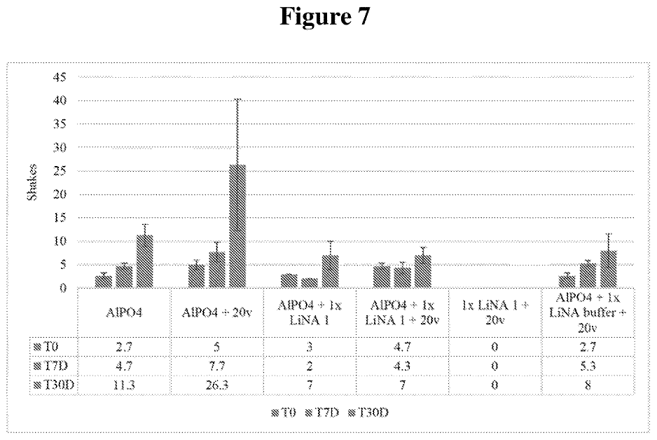

As shown in FIG. 7, the sample with AlPO₄+20vPnC took the most handshakes to resuspend with an average of 5 at TO, an average of 7.7 at T7D, and an average of 26.3 at T30D.

The addition of LiNA-1 significantly reduced the number of handshakes required to resuspend the AlPO₄+20vPnC. For example, in the case of the LiNA-1+AlPO₄+20vPnC, an average of 7 handshakes were required to resuspend the sample at T30D.

It was concluded that the addition of LiNA-1 decreased resuspension difficulty in the PFS samples containing AlPO₄ and 20vPnC. Thus, this experiment shows that similar to LiNA-2A and LiNA-2B, the addition of LiNA-1 improves the redispersion properties of the 20vPnC vaccine.

The following clauses describe additional aspects of the disclosure:
C1. A formulation comprising:
(i) at least 2, 3, 4, 5, 6, 7, 8, 9, 10, 11, 12, 13, 14, 15, 16, 17, 18, 19, or 20 different glycoconjugates;
(ii) a succinic acid or a histidine buffer having a pH in the range of 5.0 to 7.5;
(iii) calcium chloride, sodium chloride, calcium chloride and/or sodium phosphate;
(iv) a surfactant; and
(v) an adjuvant.
   C2. A formulation comprising:
(i) at least 2, 3, 4, 5, 6, 7, 8, 9, 10, 11, 12, 13, 14, 15, 16, 17, 18, 19, or 20 different glycoconjugates;
(ii) a succinic acid buffer having a pH in the range of 5.0 to 7.5;
(iii) calcium chloride;
(iv) sodium chloride;
(v) a surfactant; and
(vi) an adjuvant.
   C3. A formulation comprising:
(i) at least 2, 3, 4, 5, 6, 7, 8, 9, 10, 11, 12, 13, 14, 15, 16, 17, 18, 19, or 20 different glycoconjugates;
(ii) a succinic acid buffer having a pH in the range of 5.0 to 7.5;
(iii) sodium chloride;
(iv) sodium phosphate
(iv) a surfactant; and
(v) an adjuvant.
   C4. A formulation comprising:
(i) at least 2, 3, 4, 5, 6, 7, 8, 9, 10, 11, 12, 13, 14, 15, 16, 17, 18, 19, or 20 different glycoconjugates;
(ii) a histidine buffer having a pH in the range of 5.0 to 7.5;
(iii) sodium chloride;
(iv) a surfactant; and
(v) an adjuvant.
   C5. The formulation of any of C1-C4, comprising 20 different glycoconjugates.
   C6. A formulation comprising:
(i) at least 21 different glycoconjugates;
(ii) a succinic acid or a histidine buffer having a pH in the range of 5.0 to 7.5;
(iii) calcium chloride, sodium chloride, calcium chloride and/or sodium phosphate;
(iv) a surfactant; and
(v) an adjuvant.
   C7. A formulation comprising:
(i) at least 21 different glycoconjugates;
(ii) a succinic acid buffer having a pH in the range of 5.0 to 7.5;
(iii) calcium chloride;
(iv) sodium chloride;
(v) a surfactant; and
(vi) an adjuvant.
   C8. A formulation comprising:
(i) at least 21 different glycoconjugates;
(ii) a succinic acid buffer having a pH in the range of 5.0 to 7.5;
(iii) sodium chloride;
(iv) sodium phosphate
(iv) a surfactant; and
(v) an adjuvant.
   C9. A formulation comprising:
(i) at least 21 different glycoconjugates;
(ii) a histidine buffer having a pH in the range of 5.0 to 7.5;
(iii) sodium chloride;
(iv) a surfactant; and
(v) an adjuvant.
   C10. The formulation of any of C6-C9, comprising 21, 22, 23, 24, 25, 26, 27, 28, 29 or 30 different glycoconjugates.
   C11. The formulation of any of C6-C9, wherein the formulation comprises 24 different glycoproteins.
   C12. The formulation of any of C6-C9, wherein the formulation comprises 25 different glycoproteins.
   C13. The formulation of any of C1-C12, wherein the glycoconjugates are pneumococcal polysaccharide glycoconjugates.
   C14. The formulation of any of C1-C13, wherein the glycoconjugates comprise at least one glycoconjugate derived from S. pneumoniae serotype selected from the group consisting of S. pneumoniae serotype serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.
   C15. The formulation of any of C1-C14, wherein the glycoconjugates comprise diphtheria cross reactive material (CRM₁₉₇), Diphtheria toxin (DT), tetanus toxid (TT), C5a peptidase from Streptococcus (SCP), H. influenzae protein D (PD) or rhizavidin (CP1).
   C16. The formulation of any of C6-C15, wherein the at least 21 glycoconjugates comprise at least glycoconjugates derived from S. pneumoniae serotypes 4, 6B, 9V, 14, 18C, 19F and 23F.
   C17. The formulation of C16, wherein the S. pneumoniae serotypes are conjugated to CRM₁₉₇.
   C18. The formulation of C16, wherein the at least 25 glycoconjugates additionally comprise glycoconjugates derived from S. pneumoniae serotypes 1, 5, and 7F.
   C19. The formulation of C18, wherein S. pneumoniae serotypes 1, 4, 5, 7F, 9V and/or 23F are conjugated to PD, S. pneumoniae serotype 18C is conjugated to TT and S. pneumoniae serotype 19F is conjugated to DT.
   C20. The formulation of C16, wherein the at least 25 glycoconjugates additionally comprise glycoconjugates derived from S. pneumoniae serotypes 1, 3, 5, 6A, 7F, and 19A.
   C21. The formulation of C20, wherein the S. pneumoniae serotypes are conjugated to CRM_{197.}
   C22. The formulation of C16, wherein the at least 25 glycoconjugates additionally comprise glycoconjugates derived from S. pneumoniae serotypes 1, 3, 5, 6A, 7F, 19A. 22F and 33F.
   C23. The formulation of C22, wherein the S. pneumoniae serotypes are conjugated to CRM₁₉₇.
   C24. The formulation of C16, wherein the at least 25 glycoconjugates additionally comprise glycoconjugates derived from S. pneumoniae serotypes 1, 3, 5, 6A, 7F, 8, 10A, 11A, 12F, 15B, 19A, 22F and 33F.
   C25. The formulation of C24, wherein the S. pneumoniae serotypes are conjugated to CRM₁₉₇.
   C26. The formulation of C16, wherein the at least 25 glycoconjugates additionally comprise glycoconjugates derived from S. pneumoniae serotypes 1, 2, 3, 5, 7F, 8, 9N, 10A, 11A, 12F, 15B, 17F, 19A, 20, 22F and 33F.
   C27. The formulation of C16, wherein the at last 25 glycoconjugates additionally comprise glycoconjugates derived from S. pneumoniae serotypes 1, 2, 3, 5, 6A, 7F, 8, 9N, 10A, 11A, 12F, 15B, 17F, 19A, 20, 22F and 33F.
   C28. The formulation of C27, wherein the S. pneumoniae serotypes are conjugated to CRM₁₉₇.
   C29. The formulation of C16, wherein the at least 25 glycoconjugates additionally comprise glycoconjugates derived from S. pneumoniae serotypes 1, 2, 3, 5, 6A, 7F, 8, 9N, 10A, 11A, 12F, 15A, 15B, 19A, 22F, 23A, 23B, 24F, 33F and 35B.
   C30. The formulation of C29, wherein the S. pneumoniae serotypes are conjugated to CRM₁₉₇.
   C31. The formulation of C29, wherein S. pneumoniae serotypes 1, 2, 3, 5, 6A, 7F, 8, 9N, 10A, 11A, 12F, 15A, 15B, 19A, 22F, 23A, 23B, 24F, 33F and 35B are conjugated to CRM₁₉₇ and S. pneumoniae serotype 3 is conjugated to SCP.
   C32. The formulation of C16, wherein the at least 25 glycoconjugates additionally comprise glycoconjugates derived from S. pneumoniae serotypes 1, 3, 5, 6A, 7F, 8, 9N, 10A, 11A, 12F, 15B, 18C, 19A, 22F and 33F.
   C33. The formulation of C32, wherein at least two of the S. pneumoniae serotypes are conjugated to TT.
   C34. The formulation of C33, wherein the at least two S. pneumoniae serotypes conjugated to TT are selected from the group consisting of S. pneumoniae serotypes 1, 3, 5, 15B and 22F.
   C35. The formulation of C33, wherein at least 17 of the S. pneumoniae serotypes are conjugated to CRM₁₉₇.
   C36. The formulation of C33, wherein the at least 17 S. pneumoniae serotypes conjugated to CRM₁₉₇ are selected from the group consisting of S. pneumoniae serotypes 1, 3, 4, 5, 6A, 6B, 7F, 8, 9N, 9V, 10A, 11A, 12F, 14, 15B, 18C, 19A, 19F 22F, 23F and 33F.
   C37. The formulation of any of C1-C15, wherein the pneumococcal glyconjugates are selected from the group consisting of glycoconjugates derived from S. pneumoniae serotype 1 conjugated to CRM_{197,} S. pneumoniae serotype 3 conjugated to CRM_{197,} S. pneumoniae serotype 4 conjugated to CRM_{197,} S. pneumoniae serotype 5 conjugated to CRM_{197,} S. pneumoniae serotype 6A conjugated to CRM_{197,} S. pneumoniae serotype 6B conjugated to CRM_{197,} S. pneumoniae serotype 7F conjugated to CRM_{197,} S. pneumoniae serotype 8 conjugated to CRM_{197,} S. pneumoniae serotype 9V conjugated to CRM_{197,} S. pneumoniae serotype 10A conjugated to CRM_{197,} S. pneumoniae serotype 11A conjugated to CRM_{197,} S. pneumoniae serotype 12F conjugated to CRM_{197,} S. pneumoniae serotype 14 conjugated to CRM_{197,} S. pneumoniae serotype 15A conjugated to CRM_{197,} S. pneumoniae serotype 15B conjugated to CRM_{197,} S. pneumoniae serotype 18C conjugated to CRM_{197,} S. pneumoniae serotype 19A conjugated to CRM_{197,} S. pneumoniae serotype 19F conjugated to CRM_{197,} S. pneumoniae serotype 22F conjugated to CRM_{197,} S. pneumoniae serotype 23A conjugated to CRM_{197,} S. pneumoniae serotype 23B conjugated to CRM_{197,} S. pneumoniae serotype 23F conjugated to CRM_{197,} S. pneumoniae serotype 24F conjugated to CRM_{197,} S. pneumoniae serotype 33F conjugated to CRM_{197,} S. pneumoniae serotype 35B conjugated to CRM₁₉₇ and combinations thereof.
   C38. The formulation of any of C1-C37, wherein the total glycoconjugate concentration is in the range of 1-100 μg.
   C39. The formulation of any of C1-C38, wherein the concentration of each polysaccharide-protein conjugate is in the range of 1-10 μg.
   C40. The formulation of any of C1-C38, wherein the buffer has a concentration in the range of 1-50 mM.
   C41. The formulation of any of C1-C37, wherein the sodium chloride has a concentration of 1-300 mM.
   C42. The formulation of any of C1, C2, C6, C7, or C10-C41, wherein the calcium chloride has a concentration of 1-50 mM.
   C43. The formulation of any of C1-C3, C6-C8, or C10-C42, wherein the sodium phosphate has a concentration of 1-50 mM.
   C44. The formulation of any of C1-C41, wherein the surfactant is a polysorbate or poloxamer having a molecular weight in the range of 1100 Da to 17,400 Da.
   C45. The formulation of any of C1-C₄₄, wherein the surfactant is polysorbate 80.
   C46. The formulation of any of C1-C45, wherein the concentration of the surfactant is in the range of 0.001% to 1%.
   C47. The formulation of any of C1-C46, wherein the adjuvant is aluminum phosphate.
   C48. The formulation of any of C1-C47, wherein the concentration of the adjuvant is in the range of 0.1% to 1%.
   C49. The formulation of any of C1-C47, wherein the concentration of the adjuvant is in the range of 0.01% to 0.1%.
   C50. The formulation of any of C1-C47, wherein the concentration of the adjuvant is in the range of 0.1-1.0 mg/mL.
   C51. The formulation of any of C1-C46, wherein the adjuvant comprises aluminum phosphate at a concentration of about 0.025%.
   C52. The formulation of C1, wherein the formulation comprises 25 glycoconjugates conjugates, 5 mM succinate pH 5.8, 150 mM sodium chloride, 20 mM calcium chloride, 0.02% polysorbate 80 and 0.25 mg/ml aluminum phosphate.
   C53. The formulation of C1, wherein the formulation comprises 25 glycoconjugates, 5 mM succinate pH 5.8, 40 mM sodium phosphate, 245 mM sodium chloride, 0.02% polysorbate 80 and 0.25 mg/ml aluminum phosphate.
   C54. The formulation of C1, wherein the formulation comprises 25 glycoconjugates, 25 mM Histidine pH 5.8, 245 mM sodium chloride, 0.02% polysorbate 80 and 0.25 mg/ml aluminum phosphate.
   C55. The formulation of any of C52-C54, wherein the 25 glycoconjugates comprise glycoconjugates derived from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.
   C56. A composition comprising at least 25 different glycoconjugates and insoluble aluminum phosphate adjuvant, wherein:
at time T0 substantially all of the at least 25 different glycoconjugates are dissolved in a liquid phase or adsorbed to the insoluble aluminum adjuvant as fully dispersed liquid suspension and the at least 25 different glycoconjugates are at a concentration C0 in the liquid phase; at time T1 a portion of the at least 25 different glycoconjugates adsorbed to the insoluble adjuvant sediment from the liquid phase to form a sediment and the at least 25 different glycoconjugates are at a concentration C1 in the liquid phase;
at time T2 additional portion of the at least 25 different glycoconjugates adsorbed to the insoluble adjuvant sediment from the liquid phase to form a sediment and the at least 25 different glycoconjugates are at a concentration C2 in the liquid phase;
and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the height of a flat-bottomed cylindrical glass sample cell, collecting precipitate data every 20 μm.
   C57. The composition of C56, wherein T0 is 0 hour.
   C58. The composition of C56, wherein T1 is about 0.01 hours to 4 hours.
   C59. The composition of C58, wherein T1 is about 1 hour to 2 hours.
   C60. The composition of C56, wherein T2 is about 1 hour to 5 hours.
   C61. The composition of C60, wherein T2 is about 4 hours.
   C62. The composition of C56, wherein C0 is greater than C1 and C2.
   C63. The composition of C56, wherein C1 is greater than C2.
   C64. The composition of C56, wherein at T1 peak thickness of the sedimentation front is about 0 mm to 20 mm.
   C65. The composition of C64, wherein at T1 peak thickness of the sedimentation front is at least 2 mm.
   C66. The composition of C56, wherein at T2 peak thickness of the sedimentation front is about 2 mm to 25 mm.
   C67. The composition of C66, wherein at T2 peak thickness of the sedimentation front is at least 10 mm.
   C68. The composition of C56, wherein the sedimentary velocity of the sedimentation front is less than a peak thickness of 10 mm at about 1 hour and is greater than a peak thickness of 18 mm at about 4 hours.
   C69. The composition of C56, further comprising a time T3 wherein the insoluble aluminum phosphate adsorbed glycoconjugate sedimentation is at equilibrium with the liquid phase at T3.
   C70. The composition of C69, wherein T3 is about 2 hours to 5 hours.
   C71. The composition of C69, wherein at T3 the peak thickness of the sedimentation front is about 25 mm to 35 mm.
   C72. The composition of C56, wherein the composition has been at rest for about 1 month.
   C73. The composition of C72, wherein the composition has been at rest for at least 2 weeks.
   C74. The composition of C56, wherein the composition is stored in a container.
   C75. The composition of C74, wherein the container is a syringe.
   C76. The composition of C69, wherein after T3 the composition is resuspended with 1-10 handshakes.
   C77. The composition of C76, wherein after T3 the composition is resuspended with 1 handshake.
   C78. The composition of C56, wherein the composition comprises the formulation of any of C1 to C47.
   C79. A liquid filled container comprising at least 25 different glycoconjugates and insoluble aluminum phosphate adjuvant, wherein:
at time T0, substantially all of the at least 25 different glycoconjugates are dissolved in a liquid phase or adsorbed to the insoluble aluminum adjuvant as fully dispersed liquid suspension and the at least 25 different glycoconjugates are at a concentration C0 in the liquid phase; at time T1, a portion of the at least 25 different glycoconjugates adsorbed to the insoluble adjuvant sediment from the liquid phase to form a sediment and the at least 25 different glycoconjugates has a concentration C1 in the liquid phase;
at time T2 additional portion of the at least 25 different glycoconjugates adsorbed to the insoluble adjuvant sediment from the liquid phase to form a sediment and the at least 25 different glycoconjugates are at a concentration C2 in the liquid phase;
and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the height of a flat-bottomed cylindrical glass sample cell, collecting precipitate data every 20 μm.
   C80. The container of C79, wherein T0 is 0 hour.
   C81. The container of C79, wherein T1 is about 0.01 hours to 4 hours.
   C82. The container of C81, wherein T1 is about 1 hour to 2 hours.
   C83. The container of C79, wherein T2 is about 1 hour to 5 hours.
   C84. The c container of C83, wherein T2 is about 4 hours.
   C85. The container of C79, wherein C0 is greater than C1 and C2.
   C86. The container of C79, wherein C1 is greater than C2.
   C87. The container of C79, wherein at T1 peak thickness of the sedimentation front is about 0 mm to 20 mm.
   C88. The container of C87, wherein at T1 peak thickness of the sedimentation front is at least 2 mm.
   C89. The container of C79, wherein at T2 peak thickness of the sedimentation front is about 2 mm to 25 mm.
   C90. The container of C89, wherein at T2 peak thickness of the sedimentation front is at least 10 mm.
   C91. The container of C79, wherein the sedimentary velocity of the sedimentation front is less than a peak thickness of 10 mm at about 1 hour and is greater than a peak thickness of 18 mm at about 4 hours.
   C92. The container of C79, further comprising a time T3 wherein the insoluble aluminum phosphate adsorbed glycoconjugate sedimentation is at equilibrium with the liquid phase at T3.
   C93. The container of C92, wherein T3 is about 2 hours to 5 hours.
   C94. The container of C93, wherein at T3 the sedimentation front is about 25 mm to 35 mm.
   C95. The container of C79, wherein the container has been at rest for about 1 month.
   C96. The container of C79, wherein the container has been at rest for at least 2 weeks.
   C97. The container of C96, wherein the container is a syringe.
   C98. The container of C92, wherein after T3 the composition is resuspended with 1 to 10 handshakes.
   C99. The container of C98, wherein after T3 the composition is resuspended with 1 handshake.
   C100. The container of C79, wherein the liquid comprises the formulation of any of C1 to C47.
   C101. A composition comprising at least 25 different glycoconjugates and insoluble aluminum phosphate adjuvant, wherein:
at time T0 substantially all of the at least 25 different glycoconjugates are dissolved in a liquid phase or adsorbed to the insoluble aluminum adjuvant as fully dispersed liquid suspension and the at least 25 different glycoconjugates are at a concentration C0 in the liquid phase; at time T1 a portion of the at least 25 different glycoconjugates adsorbed to the insoluble adjuvant sediment from the liquid phase to form a sediment and the at least 25 different glycoconjugates are at a concentration C1 in the liquid phase;
at time T2 additional portion of the at least 25 different glycoconjugates adsorbed to the insoluble adjuvant sediment from the liquid phase to form a sediment and the at least 25 different glycoconjugates are at a concentration C2 in the liquid phase;
and wherein a sedimentation velocity is measured over time through static multiple light scattering to detect particle migration in the liquid, wherein a measurement head includes a pulsed near-infrared light source at a wavelength of about 880 nm and having a synchronous transmission of 180° from light source detectors and backscattering of 45° from light source detectors which move along the height of a flat-bottomed cylindrical glass sample cell, collecting precipitate data every 20 μm.
   C102. The composition of C101, wherein T0 is 0 hour.
   C103. The composition of any one of C101-C102, wherein T1 is about 0.01 hours to 4 hours after the sample reaches 45% clarification at the meniscus.
   C104. The composition of C103, wherein T1 is about 1 hour to 2 hours after the sample reaches 45% clarification at the meniscus.
   C105. The composition of C103, wherein T2 is about 1 hour to 5 hours after the sample reaches 45% clarification at the meniscus.
   C106. The composition of C103, wherein T2 is about 4 hours after the sample reaches 45% clarification at the meniscus.
   C107. The composition of any one of C101-C106, wherein C0 is greater than C1 and C2.
   C108. The composition of any one of C101-C106, wherein C1 is greater than C2.
   C109. The composition of any one of C101-C106, wherein at T1 peak thickness of the sedimentation front is about 0 mm to 20 mm.
   C110. The composition of C109, wherein at T1 peak thickness of the sedimentation front is at least 2 mm.
   C111. The composition of any one of C101-C110, wherein at T2 peak thickness of the sedimentation front is about 2 mm to 25 mm.
   C112. The composition of C111, wherein at T2 peak thickness of the sedimentation front is at least 10 mm.
   C113. The composition of C111, wherein the sedimentary velocity of the sedimentation front is less than a peak thickness of 10 mm at about 1 hour and is greater than a peak thickness of 18 mm at about 4 hours.
   C114. The composition of any one of C101-C113, further comprising a time T3 wherein the insoluble aluminum phosphate adsorbed glycoconjugate sedimentation is at equilibrium with the liquid phase at T3.
   C115. The composition of C114, wherein T3 is about 2 hours to 5 hours after the sample reaches 45% clarification at the meniscus.
   C116. The composition of C114 or C115, wherein at T3 the peak thickness of the sedimentation front is about 25 mm to 35 mm.
   C117. The composition any one of C101-C116, wherein the composition has been at rest for about 1 month.
   C118. The composition of any one of C101-C116, wherein the composition has been at rest for at least 2 weeks.
   C119. The composition any one of C101-C118, wherein the composition is stored in a container.
   C120. The composition of C119, wherein the container is a syringe.
   C121. The composition any one of C114-C120, wherein after T3 the composition is resuspended with 1-10 handshakes.
   C122. The composition of C121, wherein after T3 the composition is resuspended with 1 handshake.
   C123. The composition any one of C101-C122, wherein the composition comprises the formulation of any one of C1-C47.
   C124. An adjuvant comprising liposomes.
   C125. The adjuvant of C124, wherein the liposomes in the adjuvant range in size from between about 30 nm and about 400 nm.
   C126. The adjuvant of C124, wherein the liposomes in the adjuvant range in size from between about 30 nm and about 200 nm.
   C127. The adjuvant of C124, wherein the liposomes in the adjuvant have a size of less than about 200 nm.
   C128. The adjuvant of any one of C124-C127, wherein the liposomes in the adjuvant have a polydispersity index (PDI) between about 0.05 and about 0.5.
   C129. The adjuvant of any one of C124-C127, wherein the liposomes in the adjuvant have a polydispersity index (PDI) between about 0.05 and about 0.3.
   C130. The adjuvant of any one of C124-C127, wherein the liposomes in the adjuvant have a polydispersity index (PDI) of less than about 0.3.
   C131. The adjuvant of C124, wherein the liposomes in the range in size from between about 30 nm and about 1400 nm.
   C132. The adjuvant of C124, wherein the liposomes in the adjuvant range in size from between about 300 nm and about 1000 nm.
   C133. The adjuvant of C124, wherein the liposomes in the adjuvant have a size of more than about 300 nm.
   C134. The adjuvant of any one of C124 or C131-C133, wherein the liposomes in the adjuvant have a polydispersity index (PDI) between about 0.4 and about 1.
   C135. The adjuvant of any one of C124 or C131-C133, wherein the liposomes in the adjuvant have a polydispersity index (PDI) of more than about 0.5.
   C136. The adjuvant of any one of C124 or C131-C133, wherein the liposomes in the adjuvant have a polydispersity index (PDI) of more than about 0.4.
   C137. The adjuvant of any one of C124-C136, wherein the adjuvant comprises MPLA and a saponin.
   C138. The adjuvant of any one of C124-C136, wherein the adjuvant comprises monophosphoryl lipid A phosphorylated hexaAcyl disaccharide (PHAD®), and a saponin.
   C139. The adjuvant of any one of C124-C136, wherein the adjuvant comprises monophosphoryl 3-deacyl lipid A phosphorylated hexaacyl disaccharide (3D-PHAD®), and a saponin.
   C140. The adjuvant of any one of C124-C136, wherein the adjuvant comprises 3D-PHAD®, QS-21, 1,2-dimyristoyl-sn-glycero-3-phosphocholine (DMPC), 1,2-dimyristoyl-sn-glycero-3-phospho-(1′-rac-glycerol) (DMPG), and cholesterol.
   C141. The adjuvant of any one of C124-C136, wherein the adjuvant comprises 3D-PHAD® QS-21, DMPC, DMPG, cholesterol, phosphate buffer, and sodium chloride.
   C142. The adjuvant of any one of C124-C136, wherein the adjuvant comprises 3D-PHAD®, QS-21, DMPC, DMPG, cholesterol, 10 mM phosphate buffer, and 150 mM sodium chloride.
   C143. The adjuvant of any one of C139-C142, comprising 3D-PHAD® at a concentration between about 0.2 mg/ml and about 0.6 mg/ml.
   C144. The adjuvant of any one of C139-C142, comprising 3D-PHAD® at a concentration of about 0.4 mg/ml.
   C145. The adjuvant of any one of C139-C142, comprising 3D-PHAD® at a concentration between about 0.4 mg/ml and about 1.2 mg/ml.
   C146. The adjuvant of any one of C139-C142, comprising 3D-PHAD® at a concentration of about 0.8 mg/ml.
   C147. The adjuvant of any one of C124-C146, comprising DMPC at a concentration between about 7 mg/ml and about 21 mg/ml.
   C148. The adjuvant of any one of C124-C146, comprising DMPC at a concentration of about 14 mg/ml.
   C149. The adjuvant of any one of C124-C146, comprising DMPC at a concentration between about 14 mg/ml and about 42 mg/ml.
   C150. The adjuvant of any one of C124-C146, comprising DMPC at a concentration of about 28 mg/ml.
   C151. The adjuvant of any one of C124-C150, comprising DMPG at a concentration between about 0.8 mg/ml and about 2.4 mg/ml.
   C152. The adjuvant of any one of C124-C150, comprising DMPG at a concentration of about 1.6 mg/ml.
   C153. The adjuvant of any one of C124-C150, comprising DMPG at a concentration between about 1.6 mg/ml and about 4.8 mg/ml.
   C154. The adjuvant of any one of C124-C150, comprising DMPG at a concentration of about 3.2 mg/ml.
   C155. The adjuvant of any one of C124-C154, comprising cholesterol at a concentration between about 5 mg/ml and about 17 mg/ml.
   C156. The adjuvant of any one of C124-C154, comprising cholesterol at a concentration of about 11 mg/ml.
   C157. The adjuvant of any one of C124-C154, comprising cholesterol at a concentration between about 10 mg/ml and about 34 mg/ml.
   C158. The adjuvant of any one of C124-C154, comprising cholesterol at a concentration of about 22 mg/ml.
   C159. The adjuvant of any one of C124-C158, comprising QS-21 at a concentration of between about 0.1 mg/ml and about 0.3 mg/ml.
   C160. The adjuvant of any one of C124-C158, comprising QS-21 at a concentration of about 0.2 mg/ml.
   C161. The adjuvant of any one of C124-C158, comprising QS-21 at a concentration of between about 0.2 mg/ml and about 0.6 mg/ml.
   C162. The adjuvant of any one of C124-C158, comprising QS-21 at a concentration of about 0.4 mg/ml.
   C163. The adjuvant of any one of C124-C162, comprising DMPC and DMPG at a mole ratio of DMPC to DMPG (mol/mol) of between about 10:1 and about 8:1.
   C164. The adjuvant of any one of C124-C162, comprising DMPC and DMPG at a mole ratio of DMPC to DMPG (mol/mol) of about 9:1.
   C165. The adjuvant of any one of C124-C164, wherein the adjuvant formulation has a mole ratio of cholesterol:phospholipids of greater than 1.
   C166. The adjuvant of any one of C124-C164, wherein the adjuvant formulation has a mole ratio of cholesterol:phospholipids between about 55:50 and about 55:40.
   C167. The adjuvant of any one of C124-C164, wherein the adjuvant formulation has a mole ratio of cholesterol:phospholipids of about 55:45.
   C168. The adjuvant of any one of C124-C167, wherein the adjuvant is LiNA-2.
   C169. The adjuvant of C168, wherein the adjuvant is 0.0625 XLiNA-2, 0.125XLiNA-2, 0.25XLiNA-2, 0.5XLiNA-2, 1XLiNA-2, or 2XLiNA-2.
   C170. The adjuvant of C124, wherein the adjuvant comprises monophosphoryl lipid A phosphorylated hexaAcyl disaccharide (PHAD®) or monophosphoryl 3-deacyl lipid A phosphorylated hexaacyl disaccharide (3D-PHAD®), dioleoyl phosphatidylcholine (DOPC), cholesterol, and QS-21.
   C171. The adjuvant of C124, wherein the adjuvant comprises LiNA-1.
   C172. The formulation of any one of C1-C55, further comprising the adjuvant of any one of C124-C171.
   C173. The adjuvant or formulation of any one of C124-C172, for use in the resuspension of components of an immunogenic composition, wherein components in suspension in the immunogenic composition sediment over time.
   C174. The adjuvant or formulation of C173, wherein the immunogenic composition is stored in a container.
   C175. The adjuvant or formulation of C174, wherein the container is a syringe.
   C176. The adjuvant or formulation of any one of C173-C175, wherein at time T0 substantially all the components in the immunogenic composition are in suspension.
   C177. The adjuvant or formulation of any one of C173-C176, wherein at time T0 substantially all the components in the immunogenic composition are fully dispersed.
   C178. The adjuvant or formulation of any one of C173-C177, wherein at time T0 the immunogenic composition is substantially fully homogenous.
   C179. The adjuvant or formulation of any one of C173-C178, wherein the composition comprises or further comprises aluminum.
   C180. The adjuvant or formulation of C179, wherein the aluminum is aluminum phosphate.
   C181. The adjuvant or formulation of any one of C176-C180, wherein at time T1 between about 25% and about 100% of the components in the immunogenic composition have sedimented out of suspension.
   C182. The adjuvant or formulation of any one of C176-C180, wherein at time T1 at least about 50% of the components in the immunogenic composition have sedimented out of suspension.
   C183. The adjuvant or formulation of any one of C176-C180, wherein at time T1 at least about 90% of the components in the immunogenic composition have sedimented out of suspension.
   C184. The adjuvant or formulation of any one of C176-C180, wherein at time T1 at least about 95% of the components in the immunogenic composition have sedimented out of suspension.
   C185. The adjuvant or formulation of any one of C176-C184, wherein T1 is between about 1 and about 5 days.
   C186. The adjuvant or formulation of any one of C185, wherein T1 is about 2 days.
   C187. The adjuvant or formulation of any one of C176-C184, wherein T1 is between about 5 days and about 10 days.
   C188. The adjuvant or formulation of any one of C187, wherein T1 is about 7 days.
   C189. The adjuvant or formulation of any one of C176-C184, wherein T1 is between about 25 days and about 35 days.
   C190. The adjuvant or formulation of any one of C189, wherein T1 is about 30 days.
   C191. The adjuvant or formulation of any one of C181-C190, wherein the adjuvant reduces the number of handshakes required to resuspend the immunogenic composition at T1 by between about 1 and about 100 handshakes, or more, compared to the composition without the adjuvant.
   C192. The adjuvant or formulation of C191, wherein the adjuvant reduces the number of handshakes required to resuspend the immunogenic composition at T1 by between about 20 and about 30 handshakes, or more, compared to the composition without the adjuvant.
   C193. The adjuvant or formulation of C192, wherein the adjuvant reduces the number of handshakes required to resuspend the immunogenic composition at T1 by about 22 handshakes, compared to the composition without the adjuvant.
   C194. The adjuvant or formulation of C191, wherein the adjuvant reduces the number of handshakes required to resuspend the immunogenic composition at T1 by between about 1 and about 10 handshakes, or more, compared to the composition without the adjuvant.
   C195. The adjuvant or formulation of C194, wherein the adjuvant reduces the number of handshakes required to resuspend the immunogenic composition at T1 by about 2, about 3, about 4, about 5, about 6, about 7, or about 8, compared to the composition without the adjuvant.
   C196. The adjuvant or formulation of C191, wherein the adjuvant reduces the number of handshakes required to resuspend the immunogenic composition at T1 by between about 35 and about 45 handshakes, or more, compared to the composition without the adjuvant.
   C197. The adjuvant or formulation of C196, wherein the adjuvant reduces the number of handshakes required to resuspend the immunogenic composition at T1 by about 41 handshakes, compared to the composition without the adjuvant.
   C198. The adjuvant or formulation of C191, wherein the adjuvant reduces the number of handshakes required to resuspend the immunogenic composition at T1 by between about 15 and about 20 handshakes, or more, compared to the composition without the adjuvant.
   C199. The adjuvant or formulation of C198, wherein the adjuvant reduces the number of handshakes required to resuspend the immunogenic composition at T1 by about 19 or about 20 handshakes, compared to the composition without the adjuvant.
   C200. The adjuvant or formulation of C191, wherein the adjuvant reduces the number of handshakes required to resuspend the immunogenic composition at T1 by between about 20 and about 30 handshakes, or more, compared to the composition without the adjuvant.
   C201. The adjuvant or formulation of C200, wherein the adjuvant reduces the number of handshakes required to resuspend the immunogenic composition at T1 by about 27, about 28, or about 29 handshakes.
   C202. The adjuvant or formulation of C191, wherein the adjuvant reduces the number of handshakes required to resuspend the immunogenic composition at T1 by between about 60 and about 70 handshakes, or more, compared to the composition without the adjuvant.
   C203. The adjuvant or formulation of C202, wherein the adjuvant reduces the number of handshakes required to resuspend the immunogenic composition at T1 by about 64 handshakes, compared to the composition without the adjuvant.
   C204. The adjuvant or formulation of any one of C173-C203, wherein the resuspended immunogenic composition is homogenous.
   C205. The adjuvant or formulation of any one of C173-C204, wherein the resuspended immunogenic composition is fully dispersed.
   C206. The adjuvant or formulation of any one of C173-C205, wherein the resuspended immunogenic composition appears uniform in color.
   C207. The adjuvant or formulation of any one of C173-C206, wherein the immunogenic composition comprises a bacterial or viral immunogen.
   C208. The adjuvant or formulation of C207, wherein the immunogen comprises a protein.
   C209. The adjuvant or formulation of C207, wherein the immunogen comprises a nucleic acid.
   C210. The adjuvant or formulation of C209, wherein the immunogen comprises RNA.
   C211. The adjuvant or formulation of C207, wherein the immunogen comprises a saccharide.
   C212. The adjuvant or formulation of C207, wherein the immunogen comprises a bacterial capsular polysaccharide.
   C213. The adjuvant or formulation of C211 or C212, wherein the immunogen comprises a glycoconjugate.
   C214. The adjuvant or formulation of C213, wherein the composition comprises 2, 3, 4, 5, 6, 7, 8, 9, 10, 11, 12, 13, 14, 15, 16, 17, 18, 19, 20 21, 22, 23, 24, 25, 26, 27, 28, 29 or 30 different glycoconjugates.
   C215. The adjuvant or formulation of C214, wherein the composition comprises at least 21 different glycoconjugates.
   C216. The adjuvant or formulation of C214, wherein the composition comprises at least 25 different glycoconjugates.
   C217. The adjuvant or formulation of C214, wherein the glycoconjugates are pneumococcal polysaccharide glycoconjugates.
   C218. The adjuvant or formulation of C217, wherein the glycoconjugates comprise at least one glycoconjugate selected from the group consisting of S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, 35B and combinations thereof.
   C219. The adjuvant or formulation of C217, wherein the glycoconjugates comprise each of the following S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F, and 35B.

1.-146. (canceled)

147. An immunogenic composition comprising capsular polysaccharide of at least 25 different S. pneumoniae serotypes conjugated to a carrier protein, NaCl, optionally a second salt, a surfactant and an adjuvant.

148. The immunogenic composition of claim 147, wherein said second salt is sodium phosphate, the concentration of which ranges from about 1 mM to 50 mM, or from about 10 mM to 30 mM, or from about 15 mM to 25 mM, or from about 20 mM to 50 mM, or from about 30 mM to 50 mM, or from about 35 mM to 45 mM, or is about 20 mM, or is about 40 mM.

149. The immunogenic composition of claim 148, wherein the concentration of NaCl ranges from about 1 mM to 300 mM, or from about 50 mM to 300 mM, or from about 100 mM to 300 mM, or from about 100 mM to 200 mM, or from about 150 mM to 250 mM, or from about 200 mM to 300 mM, or is about 150 mM, or is about 245 mM.

150. The immunogenic composition of claim 147, further comprising succinate, the concentration of which ranges from about 1 mM to 50 mM, or from about 1 mM to 30 mM, or from 1 mM to 10 mM, or from about 5 mM to 9 mM, or is about 5 mM.

151. The immunogenic composition of claim 150, wherein the concentration of NaCl ranges from about 1 mM to 300 mM, or from about 50 mM to 300 mM, or from about 100 mM to 250 mM, or from about 100 mM to 200 mM, or from about 100 mM to 300 mM, or from about 150 mM to 250 mM, or from about 200 mM to 300 mM, or is about 245 mM, or is about 150 mM.

152. The immunogenic composition of claim 151, wherein said second salt is sodium phosphate, the concentration of which ranges from about 1 mM to 50 mM, or from about 10 mM to 30 mM, or from about 20 mM to 50 mM, or from about 15 mM to 25 mM, or from about 30 mM to 50 mM, or from about 35 mM to 45 mM, or is about 20 mM, or is about 40 mM.

153. The immunogenic composition of claim 151, wherein said second salt is CaCl2), the concentration of which ranges from about 1 mM to 50 mM, or from about 10 mM to 30 mM, or from about 15 mM to 25 mM, or from about 20 mM to 30 mM, or is about 20 mM.

154. The immunogenic composition of claim 151, wherein said second salt is MgCl₂, the concentration of which ranges from about about 1 mM to 50 mM, or from about 10 mM to 30 mM, or from about 10 mM to 50 mM, or from about 15 mM to 25 mM, or from about 30 mM to 50 mM, or from about 20 mM to 50 mM, or from about 35 mM to 45 mM, or is about 20 mM, or is about 40 mM.

155. The immunogenic composition of claim 147, further comprising histidine, the concentration of which ranges from about 1 mM to 50 mM, or from about 1 mM to 30 mM, or from about 10 mM to 30 mM, or from about 20 mM to 30 mM, or is about 30 mM.

156. The immunogenic composition of claim 155, wherein the concentration of NaCl ranges from about 1 mM to 300 mM, or from about 50 mM to 300 mM, or from about 100 mM to 300 mM, or from about 100 mM to 200 mM, or from about 150 mM to 250 mM, or from about 200 mM to 300 mM, or is about 150 mM, or is about 245 mM.

157. The immunogenic composition of claim 156, further comprising sodium phosphate, wherein the concentration of sodium phosphate ranges from about 1 mM to 50 mM, or from about 10 mM to 30 mM, or from about 15 mM to 25 mM, or from about 20 mM to 50 mM, or from about 30 mM to 50 mM, or from about 35 mM to 45 mM, or is about 20 mM, or is about 40 mM.

158. The immunogenic composition of claim 147, wherein the pH of said composition ranges from about pH 5.0 to pH 7.5, or from about pH 5.5 to pH 7.5, or from about pH 5.5 to pH 6.0, or from about pH 5.6 to pH 7.0, or from about pH 5.8 to pH 6.0, or is about pH 5.8.

159. The immunogenic composition of claim 147, wherein said surfactant is a polysorbate, optionally polysorbate 20 or polysorbate 80.

160. The immunogenic composition of claim 159, wherein the concentration of said surfactant ranges from about 0.001% to 1%.

161. The immunogenic composition of claim 147, wherein the concentration per dose of each serotype capsular polysaccharide ranges from about about 1.5 μg to about 5.0 μg, or is about 2.2 μg or is about 4.4 μg.

162. The immunogenic composition of claim 161, wherein the volume of a dose is about 0.5 mL.

163. The immunogenic composition of claim 147, wherein said adjuvant is an aluminum salt, optionally an aluminum salt, optionally aluminum phosphate.

164. The immunogenic composition of claim 163, wherein said adjuvant is aluminum phosphate, the concentration of which ranges from about about 0.1 mg/ml to about 1.0 mg/mL.

165. The immunogenic composition of claim 163, wherein said adjuvant is aluminum phosphate, which forms a sediment in equilibrium with the liquid phase of said composition after about 2 hours to 5 hours of settling time.

166. The immunogenic composition of claim 147, wherein after storage in a prefilled syringe for about 1 month at a temperature of about 2° C. to 8° C., said composition can be resuspended with about 1 to 10 handshakes.

167. The immunogenic composition of claim 147, wherein the sedimentation velocity of said composition is faster than a control composition comprising capsular polysaccharide of S. pneumoniae serotypes 1, 3, 4, 5, 6A, 6B, 7F, 8, 9V, 10A, 11A, 12F, 14, 15B, 18C, 19A, 19F, 22F, 23F and 33F, each individually conjugated to CRM₁₉₇, 5 mM Succinate (pH 5.8), 150 mM NaCl, 0.02% polysorbate 80, and 0.25 mg/ml aluminum as aluminum phosphate.

168. The immunogenic composition of claim 147, wherein said composition comprises capsular polysaccharide from at least one S. pneumoniae serotype selected from the group consisting of serotypes 15A, 23A, 23B, 24F, and 35B.

169. A container comprising the immunogenic composition of claim 1, optionally a prefilled syringe or a vial.


SEQUENCE LISTING (40 sequences)

SEQ ID NO: 1 - DNA, 21 bp, synthetic construct
  misc_feature 1..21: note = "A class" CpG oligonucleotide
  source 1..21: mol_type = other DNA
ggggacgacgtcgtggggggg

SEQ ID NO: 2 - DNA, 21 bp, synthetic construct
  misc_feature 1..21: note = "A class" CpG oligonucleotide
  source 1..21: mol_type = other DNA
ggggacgacgtcgtggggggg

SEQ ID NO: 3 - DNA, 21 bp, synthetic construct
  misc_feature 1..21: note = "B class" CpG oligonucleotide
  source 1..21: mol_type = other DNA
tcgtcgtttttcggtgctttt

SEQ ID NO: 4 - DNA, 21 bp, synthetic construct
  misc_feature 1..21: note = "B class" CpG oligonucleotide
  source 1..21: mol_type = other DNA
tcgtcgtttttcggtcgtttt

SEQ ID NO: 5 - DNA, 24 bp, synthetic construct
  misc_feature 1..24: note = "B class" CpG oligonucleotide
  source 1..24: mol_type = other DNA
tcgtcgttttgtcgttttgtcgtt

SEQ ID NO: 6 - DNA, 24 bp, synthetic construct
  misc_feature 1..24: note = "B class" CpG oligonucleotide
  source 1..24: mol_type = other DNA
tcgtcgtttcgtcgttttgtcgtt

SEQ ID NO: 7 - DNA, 24 bp, synthetic construct
  misc_feature 1..24: note = "B class" CpG oligonucleotide
  source 1..24: mol_type = other DNA
tcgtcgttttgtcgtttttttcga

SEQ ID NO: 8 - DNA, 21 bp, synthetic construct
  misc_feature 1..21: note = B-Class oligonucleotide
  source 1..21: mol_type = other DNA
tcgtcgtttttcggtgctttt

SEQ ID NO: 9 - DNA, 21 bp, synthetic construct
  misc_feature 1..21: note = B-Class oligonucleotide
  source 1..21: mol_type = other DNA
tcgtcgtttttcggtcgtttt

SEQ ID NO: 10 - DNA, 24 bp, synthetic construct
  misc_feature 1..24: note = B-Class oligonucleotide
  source 1..24: mol_type = other DNA
tcgtcgttttgtcgttttgtcgtt

SEQ ID NO: 11 - DNA, 24 bp, synthetic construct
  misc_feature 1..24: note = B-Class oligonucleotide
  source 1..24: mol_type = other DNA
tcgtcgtttcgtcgttttgtcgtt

SEQ ID NO: 12 - DNA, 24 bp, synthetic construct
  misc_feature 1..24: note = B-Class oligonucleotide
  source 1..24: mol_type = other DNA
tcgtcgttttgtcgtttttttcga

SEQ ID NO: 13 - DNA, 22 bp, synthetic construct
  misc_feature 1..22: note = C class CpG Oligonucleotide
  source 1..22: mol_type = other DNA
tcgcgtcgttcggcgcgcgccg

SEQ ID NO: 14 - DNA, 23 bp, synthetic construct
  misc_feature 1..23: note = C class CpG Oligonucleotide
  source 1..23: mol_type = other DNA
tcgtcgacgttcggcgcgcgccg

SEQ ID NO: 15 - DNA, 21 bp, synthetic construct
  misc_feature 1..21: note = C class CpG Oligonucleotide
  source 1..21: mol_type = other DNA
tcggacgttcggcgcgcgccg

SEQ ID NO: 16 - DNA, 19 bp, synthetic construct
  misc_feature 1..19: note = C class CpG Oligonucleotide
  source 1..19: mol_type = other DNA
tcggacgttcggcgcgccg

SEQ ID NO: 17 - DNA, 20 bp, synthetic construct
  misc_feature 1..20: note = C class CpG Oligonucleotide
  source 1..20: mol_type = other DNA
tcgcgtcgttcggcgcgccg

SEQ ID NO: 18 - DNA, 20 bp, synthetic construct
  misc_feature 1..20: note = C class CpG Oligonucleotide
  source 1..20: mol_type = other DNA
tcgacgttcggcgcgcgccg

SEQ ID NO: 19 - DNA, 18 bp, synthetic construct
  misc_feature 1..18: note = C class CpG Oligonucleotide
  source 1..18: mol_type = other DNA
tcgacgttcggcgcgccg

SEQ ID NO: 20 - DNA, 18 bp, synthetic construct
  misc_feature 1..18: note = C class CpG Oligonucleotide
  source 1..18: mol_type = other DNA
tcgcgtcgttcggcgccg

SEQ ID NO: 21 - DNA, 22 bp, synthetic construct
  misc_feature 1..22: note = C class CpG Oligonucleotide
  source 1..22: mol_type = other DNA
tcgcgacgttcggcgcgcgccg

SEQ ID NO: 22 - DNA, 22 bp, synthetic construct
  misc_feature 1..22: note = C class CpG Oligonucleotide
  source 1..22: mol_type = other DNA
tcgtcgttttcggcgcgcgccg

SEQ ID NO: 23 - DNA, 22 bp, synthetic construct
  misc_feature 1..22: note = C class CpG Oligonucleotide
  source 1..22: mol_type = other DNA
tcgtcgttttcggcggccgccg

SEQ ID NO: 24 - DNA, 24 bp, synthetic construct
  misc_feature 1..24: note = C class CpG Oligonucleotide
  source 1..24: mol_type = other DNA
tcgtcgttttacggcgccgtgccg

SEQ ID NO: 25 - DNA, 23 bp, synthetic construct
  misc_feature 1..23: note = C class CpG Oligonucleotide
  source 1..23: mol_type = other DNA
tcgtcgttttcggcgcgcgccgt

SEQ ID NO: 26 - DNA, 22 bp, synthetic construct
  misc_feature 1..22: note = C-Class oligonucleotides
  source 1..22: mol_type = other DNA
tcgcgtcgttcggcgcgcgccg

SEQ ID NO: 27 - DNA, 23 bp, synthetic construct
  misc_feature 1..23: note = C-Class oligonucleotide
  source 1..23: mol_type = other DNA
tcgtcgacgttcggcgcgcgccg

SEQ ID NO: 28 - DNA, 21 bp, synthetic construct
  misc_feature 1..21: note = C-Class oligonucleotide
  source 1..21: mol_type = other DNA
tcggacgttcggcgcgcgccg

SEQ ID NO: 29 - DNA, 19 bp, synthetic construct
  misc_feature 1..19: note = C-Class oligonucleotide
  source 1..19: mol_type = other DNA
tcggacgttcggcgcgccg

SEQ ID NO: 30 - DNA, 20 bp, synthetic construct
  misc_feature 1..20: note = C-Class oligonucleotide
  source 1..20: mol_type = other DNA
tcgcgtcgttcggcgcgccg

SEQ ID NO: 31 - DNA, 20 bp, synthetic construct
  misc_feature 1..20: note = C-Class oligonucleotide
  source 1..20: mol_type = other DNA
tcgacgttcggcgcgcgccg

SEQ ID NO: 32 - DNA, 18 bp, synthetic construct
  misc_feature 1..18: note = C-Class oligonucleotide
  source 1..18: mol_type = other DNA
tcgacgttcggcgcgccg

SEQ ID NO: 33 - DNA, 18 bp, synthetic construct
  misc_feature 1..18: note = C-Class oligonucleotide
  source 1..18: mol_type = other DNA
tcgcgtcgttcggcgccg

SEQ ID NO: 34 - DNA, 22 bp, synthetic construct
  misc_feature 1..22: note = C-Class oligonucleotide
  source 1..22: mol_type = other DNA
tcgcgacgttcggcgcgcgccg

SEQ ID NO: 35 - DNA, 22 bp, synthetic construct
  misc_feature 1..22: note = C-Class oligonucleotide
  source 1..22: mol_type = other DNA
tcgtcgttttcggcgcgcgccg

SEQ ID NO: 36 - DNA, 22 bp, synthetic construct
  misc_feature 1..22: note = C-Class oligonucleotide
  source 1..22: mol_type = other DNA
tcgtcgttttcggcggccgccg

SEQ ID NO: 37 - DNA, 24 bp, synthetic construct
  misc_feature 1..24: note = C-Class oligonucleotide
  source 1..24: mol_type = other DNA
tcgtcgttttacggcgccgtgccg

SEQ ID NO: 38 - DNA, 23 bp, synthetic construct
  misc_feature 1..23: note = C-Class oligonucleotide
  source 1..23: mol_type = other DNA
tcgtcgttttcggcgcgcgccgt

SEQ ID NO: 39 - DNA, 23 bp, synthetic construct
  misc_feature 1..23: note = P class CpG oligonucleotide
  source 1..23: mol_type = other DNA
tcgtcgacgatcggcgcgcgccg

SEQ ID NO: 40 - DNA, 23 bp, synthetic construct
  misc_feature 1..23: note = P class CpG oligonucleotide
  source 1..23: mol_type = other DNA
tcgtcgacgatcggcgcgcgccg